(19) United States
(12) Patent Application Publication
(10) Pub. No.: US 2025/0327078 A1
(43) Pub. Date: Oct. 23, 2025

(54) Compositions for and Methods Creating Exon-Skipped mRNA Producing Internal Controls

(71) Applicants: Shift Pharmaceuticals Holding Inc., Columbia, MO (US); The Curators of the University of Missouri, Columbia, MO (US)

(72) Inventors: Stephen D. O'Connor, Columbia, MO (US); Christian Lorson, Columbia, MO (US)

(21) Appl. No.: 18/855,519

(22) PCT Filed: Apr. 14, 2023
(86) PCT No.: PCT/US2023/065761

§ 371 Date: Oct. 9, 2024

Related U.S. Application Data
(60) Provisional application No. 63/331,044, filed on Apr. 14, 2022.
(60) Provisional application No. 63/331,045, filed on Apr. 14, 2022.

(51) Int. Cl. C12N 15/113; C12Q 1/6883

(52) U.S. Cl. CPC C12N 15/1136; C12Q 1/6883; C12N 2310/14; C12N 2310/3233; C12N 2320/33; C12Q 2600/158

(57) ABSTRACT

Disclosed herein are a new class of therapeutics and methods that can elicit a molecular response in cells and subjects where both the native unaffected mRNA (full-length mRNA) and drug-induced “exon-skipped mRNA” can be monitored during the course of treatment and in certain embodiments related to downstream protein production. This approach allows for careful monitoring of the effect of the drugs proposed during in vitro and animal studies, and in actual patients during clinical trials and patient deployment.

### REFERENCE TO SEQUENCE LISTING SUBMITTED ELECTRONICALLY

This international application claims the benefit of U.S. Provisional Application 63/331,044, filed on Apr. 14, 2022 and U.S. Provisional Application 63/331,045, filed on Apr. 14, 2022, both of which are incorporated herein in their entireties.

### REFERENCE TO SEQUENCE LISTING SUBMITTED ELECTRONICALLY

The contents of the electronically submitted sequence listing in ST26 format (Name UMC_226825.xml; Size: 242,822 bytes; and Date of Creation: Apr. 13, 2023) filed with this application is incorporated herein by reference in its entirety.

### BACKGROUND

This disclosure is directed to the field of modulating gene expression, mRNA production, and protein production as well as treating certain diseases. Certain compositions and methods can be used for research and therapeutic purposed.

Many disease states in humans are caused by low levels of functional protein within a specific tissue or the body. Initially, antibody production approaches were used to synthesize functional proteins (or partial proteins) in a manufacturing setting then injected into humans to improve patient health. More recently, small molecule and synthetic RNA or DNA methods have been used to stimulate protein production within a patient using the patient's own protein production pathways. Measurement of the effect of these drugs within an animal model or patient have been relatively straightforward, since assays to monitor the molecular effect are “positive result assays”. By positive result, it is meant that the compounds added to the animal produce a molecule that is not present within the subject (or are present at a very low level) so the baseline levels of the assay measurement is very low (or zero) and the assay measures a positive increase in the presence of that molecule with drug treatment. For these approaches, the sensitivity of the measurement is limited mainly be the sensitivity of the assay, not the internal biological variance of the tissue or animal. The response to the compound of interest can vary from subject to subject, but the measurement is not limited to the same degree by biological variance since the product that is being probed or assays for is not present prior to the compound addition.

For many of the drugs that have been developed that “increase” the amount of functional protein within the body, an upper threshold on the amount of functional protein created or added is not an issue, since more functional protein is desirable and generally will have a positive effect on the patient's health. Thus, the overall goal of the addition of the drug is to create as much functional protein as possible. The amount of drug added is typically limited at a higher level based on the potential side effects of the drug and/or the cost of the drug being manufactured. For other drugs that require more difficult to achieve delivery methods (such as IV delivery, intrathecal injection, etc.), dosing realities may come into play, since the patient cannot realistically perform these procedures on their own or receive them every day unless hospitalized. In practice, for therapeutics in this sector, creating as much functional protein as possible (while maintaining no side effects) is the overall goal.

For example, Spinraza (aka Nusinersen; Pao P W, Wee K B, Yee W C, Pramono Z A, Dwipramono Z A (April 2014). “Dual masking of specific negative splicing regulatory elements resulted in maximal exon 7 inclusion of SMN2 gene”. Molecular Therapy. 22 (4): 854-61. doi:10.1038/mt.2013.276. PMC 3982506. PMID 24317636; Gene Therapy. 24 (9): 520-526. doi:10.1038/gt.2017.34. PMC 5623086. PMID 28485722) developed by Ionis Pharmaceuticals using a 5′methoxy anti-sense oligonucleotide to block a repressor region of the RNA and promote exon inclusion in the production of full-length SMN proteins. This treatment is approved and used for patients with Spinal Muscular Atrophy and the upper dosing limit is defined by the safety/toxicity profile of the drug, not by how much function SMN protein is synthesized in the cells (for Spinal Muscular Atrophy, more SMN protein is better). Eteplirsen from Sarepta Therapeutics has a similar approach for Duchene Muscular Dystrophy. This drug works by binding to the patients RNA to silence exon 51 of the dystrophin gene and restore functional dystrophin protein. Again, in this disease, the more function protein the better. Other examples of approaches where the desired goal is to create as much functional protein as possible are extensively described in the literature.

For compounds and drugs that are designed to affect animal cells at the molecular DNA or RNA level, sensitivity of the measurement assay is often not the limiting factor due to the elegant sensitivity and specificity of oligonucleotide amplification techniques such as PCR or other amplification techniques know to those of ordinary skill in the art. These techniques, when properly optimized, can detect extremely low levels of a specific nucleic acid sequence present in a complicated tissue or fluid samples. Rather, determining the change of the RNA or DNA in the animal tissue caused by the activity of the applied drug relative to the naturally occurring background changes in the oligonucleotide is often the limiting affect. Numerous other biological techniques are known to one skilled in the art for detecting low levels of nucleic acids or proteins, and included but are not limited to PCR, other nucleic acid amplification techniques, binding capture techniques such as ELISA assays, liquid chromatography followed by mass spectrometry, nucleic acid sequencing, etc.

Certain drug approaches have been pursued and published that target diseases where the molecular mode of action is to lower, rather than raise, the total naturally-occurring protein present in a patient by blocking messenger RNA (mRNA) within a patients cells, thus lowering the patients protein levels. Numerous approaches have been described to “silence” the mRNA using anti-sense oligonucleotides, small molecule binding entities, miRNAs, siRNAs, etc.

In many cases, it is most desirable to target a specific goal of protein production and it would be highly desirable to alter the subjects dosing schedule to achieve that goal if possible. Dosing requirements may be highly dependent on a number of patient factors including weight, age, disease progression, lifestyle, etc. For example, previously described approaches for treating CMT1A (and other monogenic and multi-genetic) diseases do not allow an accurate methodology for tracking the mRNA production and thus total (or partial) protein production from that mRNA.

Thus, compounds and testing methods are needed to addressed disease states or conditions where it is desirable to lower total RNA and thus protein production in a quantitative manner even at relatively low levels of changes of protein being desired. Additionally, it is extremely desirable to be able to monitor the actual response of a given patient to a drug at the molecular level in order to change therapeutic dosing over time and manage protein production (and thus disease status and or progression) as well as possible.

### SUMMARY

Provided for herein is a composition comprising a compound that specifically targets a pre-mRNA to induce production of an exon-skipped mRNA via exon-skipping. In certain embodiments, the exon-skipped mRNA produced is detectable such as by a variety of nucleic acid detection assays. In certain embodiments, the exon-skipped mRNA induced by the compound is also produced at a background level in a cell, organism, subject, patient, etc., and the exon-skipped mRNA induced by the compound is measurable above such background level. In certain embodiments, the exon-skipped mRNA induced by the compound is not produced in a cell, organism, subject, patient, etc., absent the compound, for example wherein the exon-skipped mRNA is a non-naturally occurring mRNA. In certain embodiments in addition to inducing production of an exon-skipped mRNA, the compound reduces the amount the of the corresponding full-length mRNA expressed in a cell. In certain embodiments, the compound reduces, but does not completely abolish, the amount of the corresponding full-length mRNA expressed in the cell.

In certain embodiments, the exon-skipping inducing compound of this disclosure specifically that targets the pre-mRNA is an antisense oligonucleotide (ASO). In certain embodiments, the ASO is a phosphorodiamidate morpholino oligomer (PMO). And, in certain embodiments, at least one of the sugars in the nucleic acid backbone of the ASO is 2′-OMe-substituted.

In certain embodiments, an ASO of this disclosure comprises or consists of a complementary region that is at least 70%, 75%, 80%, 85%, 90%, 95%, 96%, 97%, 98%, 99%, or 100% complementary to a target region of the pre-mRNA. In certain embodiments an ASO of this disclosure comprises or consists of a complementary region that has 1, 2, 3, 4, or 5 mismatches to a target region of the pre-mRNA. In certain embodiments, the target region of the pre-mRNA spans an intron/exon junction of one of the coding exons. Thus, in certain embodiments, binding in a cell of the complementary region of the ASO to the target region of the pre-mRNA results in exon skipping of an exon during RNA transcription.

In certain embodiments, the target pre-mRNA of the compound is associated with a disease. In certain embodiments, the composition is a therapeutic composition comprising a pharmaceutically acceptable carrier or diluent. And, in certain embodiments, the compound is an ASO that is administered in the form of a pharmaceutically acceptable salt.

Also provided for herein is a method of measuring the amount of an exon-skipped mRNA produced in response to an exon-skipping inducing compound. In certain embodiments, the amount of the exon-skipped mRNA produced is measured against the amount of an internal control. The method first comprises administering a composition comprising a compound that induces pre-mRNA exon-skipping to a cell to induce exon-skipping of a target pre-mRNA and production of the exon-skipped mRNA. In certain embodiments, the exon-skipping inducing compound reduces the amount of corresponding full-length mRNA expressed from the target pre-mRNA. In certain embodiments, the exon-skipping inducing compound reduces, but does not completely abolish, the amount of corresponding full-length mRNA expressed from the target pre-mRNA. The method next comprises obtaining a sample comprising the exon-skipped mRNA. In certain embodiments, the sample also comprises the corresponding full-length mRNA. In certain embodiments, the sample comprises the cell. The method next comprises measuring in the sample the amount of the exon-skipped mRNA. In certain embodiments, the method also comprises measuring the amount of the corresponding full-length mRNA in the sample and comparing the amount of the exon-skipped mRNA to the amount of the full-length mRNA. In certain embodiments, the cell is in a subject, the composition is administered to said subject, and the sample is a biological sample from said subject.

Also provided herein is a method of adjusting the dosing of an exon-skipping inducing compound. The method first comprises administering a dose of a composition comprising a compound that induces pre-mRNA exon-skipping to a cell to induce exon-skipping of a target pre-mRNA and production of an exon-skipped mRNA therefrom. In certain embodiments, the compound reduces the amount of corresponding full-length mRNA expressed from the target pre-mRNA. In certain embodiments, the compound reduces, but does not completely abolish, the amount of corresponding full-length mRNA expressed from the target pre-mRNA. The method then comprises obtaining a sample comprising the exon-skipped mRNA and the corresponding full-length mRNA. In certain embodiments, the sample comprises the cell. The method then comprises measuring in the sample the amount of the exon-skipped mRNA and the amount of the corresponding full-length mRNA. In certain embodiment the method then comprises determining the ratio between the amount of exon-skipped mRNA and the amount of full-length mRNA. And, in certain embodiments the method comprises adjusting the dosing of the composition to be administered based on the ratio between the amount exon-skipped mRNA and the amount of full-length mRNA. In certain embodiments the amount of the dose is adjusted and/or the frequency of administration of the dose is adjusted. Certain embodiments further comprise subsequently administering the composition to the same cell or to another cell according to the adjustment to the dosing based on the ratio between the amount exon-skipped mRNA and the amount of full-length mRNA that has been determined. In certain embodiments, the cell is in a subject, the composition is administered to said subject, and the sample is a biological sample from said subject.

Also provided for herein is a method of treating a disease or medical condition with an exon-skipping inducing compound. The method first comprises administering to a subject in need of treatment a dose of a composition comprising a compound that induces pre-mRNA exon-skipping to induce exon-skipping of a target pre-mRNA and production of an exon-skipped mRNA therefrom. In certain embodiments, the compound reduces the amount of full-length mRNA expressed from the target pre-mRNA. In certain embodiments, the compound reduces, but does not abolish, the amount of full-length mRNA expressed from the target pre-mRNA. The method next comprises obtaining a biological sample from the subject and measuring in the sample the amount of the exon-skipped mRNA. In certain embodiments, the amount of the full-length mRNA in the sample is measured. Certain embodiments further comprise determining the ratio between the amount of exon-skipped mRNA and the amount of corresponding full-length mRNA. In certain embodiments, the dosing of the composition is adjusted to be subsequently administered based on the ratio between the amount exon-skipped mRNA and the amount of full-length mRNA. In certain embodiments, the composition is subsequently administering to the same subject or to another subject according to said adjustment to the dosing based on the ratio between the amount exon-skipped mRNA and the amount of full-length mRNA.

In certain embodiments of any of the methods disclosed herein in which an exon-skipped mRNA is produced, the biological sample is a cell, tissue, organ, or a sample obtained therefrom. In certain embodiments, the biological sample is blood, plasma, cerebrospinal fluid (CSF), lymph, skin, saliva, mucus, feces, urine, eye fluid, saliva, stomach fluid, or a sample obtained therefrom.

In certain embodiments of any of the methods disclosed herein in which an exon-skipped mRNA is produced, the amount of exon-skipped mRNA and/or the amount of full-length mRNA is measured using polymerase chain reaction (PCR), nucleic acid sequencing, oligonucleotide ELISA, and/or mass spectrometry.

In certain embodiments of any of the methods disclosed herein in which an exon-skipped mRNA is produced, the administered composition comprises a pharmaceutically acceptable carrier or diluent.

In certain of any of the methods disclosed herein in which an exon-skipped mRNA is produced, the administration of the composition reduces the amount of full-length protein and/or functional protein produced from a gene of the target pre-mRNA. In certain embodiments, the method further comprises measuring the amount of the protein including the full-length protein and/or a modified protein.

### BRIEF DESCRIPTION OF THE DRAWINGS

FIG. 1 shows the overall biochemical process of molecules proceeding from: (i) genes (made up of DNA); (ii) to pre-mRNA (made up of RNA); (iii) to mRNA (made up of RNA); (iv) to proteins (made up of amino acids coded by the information contained in the mRNA).

FIG. 2 shows the variance of amount of RNA present in a given cell or tissue type over time due to natural variation. The RNA can natural achieve a high point (i) or a low point (ii) due to a variety of natural or behavior factors that may or may not be associated with a disease state.

FIG. 3 shows one example of the natural fluctuations of RNA and/or protein within a given tissue type before and after the dosing a drug at time 31 days. Concentration units are relative. A similar trend could be observed for different time points (such as hours, minutes, weeks, etc.). For both levels prior to the initiation of a drug at time 30, there is a large natural variance of the levels. Circles show “random” timepoint that might be taken to monitor each level if it is undesirable or impractical to take all of the time points shown.

FIG. 4 shows a schematic of the present invention where a drug is added (*) that produces an exon skipping even when the pre-mRNA is converted to mRNA. This drug (*) results in the production of both full-length mRNA and exon-skipped mRNA. Both of the mRNAs can be quantitated from an animal sample, either using tissue material or biological fluid. The exon-skipped mRNA produces a shortened or unstable protein which has less biological function than the full-length protein.

FIG. 5 shows one example of the data that can be generated from the addition of the drug as described in the present invention. mRNA is monitored in one or more animals is shown at natural levels prior to the addition of the drug at day 31. On the same plot (left handed Y-axis denotes relative concentration) is shown the presence of the exon-skipped mRNA. The ratio of the data from the full-length pre-mRNA (top line) and exon-skipped mRNA (bottom line) is also shown and denoted at 20% activity.

FIG. 6 schematically shows the presence of mistakes and degraded mRNA that is present when no drug is added and the presence of both these mistake and degradations as well as exon-skipped mRNA when a drug is added.

FIG. 7 shows PMP22 genetic pre-mRNA (top), resulting full-length mRNA that contains all of the amino acid coding Exons (middle) and functional protein (bottom). Introns between each exon are shown as double horizontal lines and the base pair sequences are removed during splicing. Introns between each exon are shown as double horizontal lines and the base pair sequences are removed during splicing.

FIG. 8 shows PMP22 genetic pre-mRNA (top), the addition of an ASO drug that produced an exon skipping event at Exon 3, the resulting full-length mRNA that contains all of the amino acid coding Exons except the sequences of Exon 3, and non-functioning or semi-functional protein (bottom) that is coded from the incomplete PMP22 genome. Introns between each exon are shown as double horizontal lines and the base pair sequences are removed during splicing.

FIG. 9 shows the PCR results of 6 cellular assays performed in the presence of an Exon 3 skipping drug (SHC-012) run at different conditions but the same amount of SHC-012.

FIG. 10A,B. FIG. 10 shows the sequence of PMP22 gene (SEQ ID NO: 1) around the 5′- and 3′-regions of Exon 3 (all caps) and associated upstream and downstream introns. 10A shows the 5′-end of the exon and 10B shows the 3′-end. A number of different representative 25-mer oligonucleotides ASO sequences of the present disclosure are shown (below the sequence at the top) that may produce a skipping of Exon 3 during conversion from pre-mRNA to mRNA. Various methods of synthesizing ASO are available (both solid phase and solution phases and, e.g., with 5′ to 3′ directionality or with 3′ to 5′ directionality) to one of ordinary skill in the art as long as the final ASO has the sequence disclosed (or including any mismatches as provided for herein) and hybridizes to the target region or a portion of the target region. Six representative morpholino anti-sense oligo sequences that were designed (and synthesized) to bind to the intro/exon junctions are shown where data is presented. 10A: SEQ ID NOs: 2-34; SHC-006 25-mer (SEQ ID NO: 71); SHC-001 24-mer (SEQ ID NO: 72); SHC-005 25-mer (SEQ ID NO: 73). 10B: SEQ ID NOs: 35-70; SCH-010 21-mer (SEQ ID NO: 74); SCH-012 20-mer (SEQ ID NO: 75).

FIG. 11A,B. FIG. 11 shows the sequence of PMP22 gene (SEQ ID NO: 1) around the 5′- and 3′-regions of Exon 4 (all caps) and associated upstream and downstream introns. 11A is the 5′-end of the exon and 11B is the 3′-end. A number of different 25-mer oligonucleotides ASO sequences of this disclosure are shown (below the sequence at the top) that may produce a skipping of Exon 4 during conversion from pre-mRNA to mRNA. Six representative morpholino anti-sense oligo sequences that were designed (and synthesized) to bind to the intro/exon junctions are shown where data is presented. 11A: SEQ ID NOs: 76-110; SHC-029 21-mer (SEQ ID NO: 146); SHC-028 20-mer (SEQ ID NO: 147); SHC-027 20-mer (SEQ ID NO: 148). 11B: SEQ ID NOs: 111-145; SCH-031 21-mer (SEQ ID NO: 149); SCH-030 20-mer (SEQ ID NO: 150); SCH-032 20-mer (SEQ ID NO: 151).

FIG. 12 shows the PCR results of various compounds that induced Exon 3 skipping in cellular assays. The data below each gel shows the quantitation comparison of the results using the traditional methodology (Comparison to full-length) and the comparison using one aspect of the present invention (ratio of the full-length and exon skipped mRNA). SHC-006 25-mer (SEQ ID NO: 71). SHC-001 24-mer (SEQ ID NO: 72). SHC-033 21-mer, which is in the middle of an exon and does not overlap the intron/exon region (SEQ ID NO: 239; GAAGAGGTGCTACAGTTCTGC).

FIG. 13 shows the PCR results of various compounds that induced Exon 4 skipping in cellular assays. The data below each gel shows the quantitation comparison of the results using the traditional methodology (Comparison to full-length) and the comparison using one aspect of the present invention (ratio of the full-length and exon skipped mRNA). SHC-027 20-mer (SEQ ID NO: 148). SCH-030 20-mer (SEQ ID NO: 150). SCH-032 20-mer (SEQ ID NO: 151).

FIG. 14 shows RT-PCR from C3 mice (liver) treated with SHC-012 (SEQ ID NO: 75), a scramble (2×) ASO, or a water control. Human full-length PMP22 mRNA is detectable in addition to the GAPDH control. SHC-012-induced exon 3 skipping (confirmed by sequence) is present as marked.

FIG. 15 shows the PCR results (and corresponding change in amount of full-length PMP22 mRNA) for a number of tissues after a single subcutaneous injection of SHC-012 (SEQ ID NO: 75) was given and the animals sacrificed 2 days later. The data in FIG. 15 represents percent remaining. Specifically, for each tissue type we quantitated the total amount of PMP22 full-length mRNA per tissue type and compared to the SHC-012 animals. 20% remaining indicates the 80% of the pre-mRNA was blocked from making full-length mRNA and made exon-skipped mRNA instead.

FIG. 16 shows the amount of time taken to walk across the dowel apparatus described for different treatment groups (using SHC-012; SEQ ID NO: 75), scramble animals, and wild type mice. For all groups, 3 animals were in each group and the histogram shows the average values and standard deviations. P values were calculated (to determine confidence levels against the scramble control) for each treatment group. For each group, p<0.05. All data is for average values of all animals that are 12 weeks old.

FIG. 17 shows the amount of time spent on a rotarod apparatus for the scramble, wild type, and treatment groups first injected at 5-weeks of age. For all groups, 3 animals were in each group and the histogram shows the average values and standard deviations. P values were calculated (to determine confidence levels against the scramble control) for each treatment group and are shown. All data is for average values of all animals that are 12 weeks old.

FIG. 18 shows the 5′- and 3′-ends of Exon 3 (top) and 3 different ASO compounds that were designed and tested that bridge the target regions (shown for each). These ASO target regions of the Exon that are non-continuous and will have a different effect on the three dimensional structure of the pre-mRNA than those described above. SEQ ID NOs: 152-162 and SEQ ID NOs: 233-238; SHC-043 (SEQ ID NO: 156); SHC-044 (SEQ ID NO: 159); SHC-045 (SEQ ID NO: 162); SHC-046 (SEQ ID NO: 235); SHC-047 (SEQ ID NO: 238).

FIG. 19 shows the results from PCR amplification followed by gel electrophoresis analysis of the selected Exon 3 skipping ASOs from FIG. 18.

FIG. 20A,B. FIG. 20 shows the sequence of PMP22 gene around the 5′- and 3′-regions of Exon 2 (all caps) and associated upstream and downstream introns. 20A is the 5′-end of the exon and 20B is the 3′-end. 16A: SEQ ID NOs: 163-197. 16B: SEQ ID NOs: 198-232.

FIG. 21A-D. FIG. 21 shows a schematic of a number of strategies for biding to the pre-mRNA to induce exon skipping.

FIG. 22 shows images of the sciatic nerve for WT, untreated, and treated animals (top) and images from the peroneal portion of the nerve (bottom).

FIG. 23 shows a TEM image at higher magnification of portions of a Peroneal nerve.

FIG. 24 shows electrophysiology plots from the sciatic gastric section of sedated mice (top). FIG. 19 (bottom) shows the average values of MUNE and CMAP from 3 measured animals per group.

FIG. 25 shows results for treatment groups (with the scramble animals' group and wild-type animals shown for comparison at 4 months of age). Each data set of the histogram is an average of 4 days dowel travers time at the end of each month.

FIG. 26 shows the results of the dowel traverse time experiment (with 3 month old wild-type animals also plotted for comparison).

### DETAILED DESCRIPTION

### Definitions

To the extent necessary to provide descriptive support, the subject matter and/or text of the appended claims is incorporated herein by reference in their entirety.

It will be understood by all readers of this written description that the exemplary embodiments described and claimed herein may be suitably practiced in the absence of any recited feature, element or step that is, or is not, specifically disclosed herein.

Unless defined otherwise, technical and scientific terms used herein have the same meaning as commonly understood by one of ordinary skill in the art to which this disclosure is related.

It is to be noted that the term “a” or “an” entity refers to one or more of that entity; for example, “a compound,” is understood to represent one or more compounds. As such, the terms “a” (or “an”), “one or more,” and “at least one” can be used interchangeably herein.

Furthermore, “and/or” where used herein is to be taken as specific disclosure of each of the specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Numeric ranges are inclusive of the numbers defining the range. Even when not explicitly identified by “and any range in between,” or the like, where a list of values is recited, e.g., 1, 2, 3, or 4, the disclosure specifically includes any range in between the values, e.g., 1 to 3, 1 to 4, 2 to 4, etc.

The headings provided herein are solely for ease of reference and are not limitations of the various aspects or aspects of the disclosure, which can be had by reference to the specification as a whole. Accordingly, the terms defined immediately below are more fully defined by reference to the specification in its entirety.

As used herein, the term “identity,” e.g., “percent identity” to an amino acid sequence or to a nucleotide sequence disclosed herein refers to a relationship between two or more nucleotide sequences or between two or more amino acid sequences. When a position in one sequence is occupied by the same nucleic acid base or amino acid in the corresponding position of the comparator sequence, the sequences are said to be “identical” at that position. The percentage “sequence identity” is calculated by determining the number of positions at which the identical nucleic acid base or amino acid occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. In order to optimally align sequences for comparison, the portion of a nucleotide or amino acid sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using, e.g., the program “BLAST” which is available from the National Center for Biotechnology Information, and which program incorporates the programs BLASTN (for nucleotide sequence comparison) and BLASTP (for amino acid sequence comparison), which programs are based on the algorithm of Karlin and Altschul (Proc. Natl. Acad. Sci. USA 90(12):5873-5877, 1993).

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology, but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

By an “isolated” polypeptide or a fragment, variant, or derivative thereof or the like is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are recombinant polypeptides that have been separated, fractionated, or partially or substantially purified by any suitable technique.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). As used herein, an “oligonucleotide” refers a polynucleotide of up to about 50 nucleotides or base pairs in length. The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “coding region” is a portion of nucleic acid comprising codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode a selection marker gene and a gene of interest. In addition, a vector, polynucleotide, or nucleic acid can encode heterologous coding regions, either fused or unfused to a nucleic acid encoding a polypeptide subunit or fusion protein as provided herein. Heterologous coding regions include without limitation specialized elements or motifs, such as a secretory signal peptide or a heterologous functional domain.

As used herein, an “exon” refers to the portion of a DNA or RNA sequence that results in the synthesis of an amino acid sequence.

As used herein, an “intron” refers to the portion of a DNA or RNA sequence that does not result in the synthesis of an amino acid sequence

In certain aspects, the polynucleotide or nucleic acid is DNA. In the case of DNA, a polynucleotide comprising a nucleic acid that encodes a polypeptide normally can include a promoter and/or other transcription or translation regulatory elements operably associated with one or more coding regions. An operable association or linkage can be when a coding region for a gene product, e.g., a polypeptide, can be associated with one or more regulatory sequences in such a way as to place expression of the gene product under the influence or control of the regulatory sequence(s). Two DNA fragments (such as a polypeptide coding region and a promoter associated therewith) can be “operably associated” or “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Other transcription regulatory elements, besides a promoter, for example enhancers, operators, repressors, and transcription termination signals, can be operably associated with the polynucleotide to direct cell-specific transcription.

A variety of transcription regulatory regions are known to those skilled in the art. These include, without limitation, transcription regulatory regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription regulatory regions include those derived from vertebrate genes such as actin, heat shock protein, bovine growth hormone and rabbit beta-globin, as well as other sequences capable of controlling gene expression in eukaryotic cells. Additional suitable transcription regulatory regions include tissue-specific promoters and enhancers.

Similarly, a variety of translation regulatory elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other aspects, a polynucleotide can be RNA, for example, in the form of a pre-mRNA or messenger RNA (mRNA).

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain aspects, the native signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse ß-glucuronidase.

A “vector” is nucleic acid molecule as introduced into a host cell, thereby producing a transformed host cell. A vector can include nucleic acid sequences that permit it to replicate in a host cell, such as an origin of replication. A vector can also include one or more selectable marker gene and other genetic elements known in the art. Illustrative types of vectors include plasmids, phages, viruses and retroviruses.

A “transformed” cell, or a “host” cell, is a cell into which a nucleic acid molecule has been introduced by molecular biology techniques. As used herein, the term transformation encompasses those techniques by which a nucleic acid molecule can be introduced into such a cell, including transfection with viral vectors, transformation with plasmid vectors, and introduction of naked DNA by electroporation, lipofection, and particle gun acceleration. A transformed cell or a host cell can be a bacterial cell or a eukaryotic cell.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into pre-mRNA and messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

The term “pharmaceutical composition” refers to a preparation or mixture of substances suitable for administering to a subject, i.e., that is in such form as to permit the biological activity of the active ingredient to be effective, and that contains no additional components that are unacceptably toxic to a subject to which the composition would be administered. Such composition can be sterile. For example, a pharmaceutical composition may comprise an oligomeric compound and a sterile aqueous solution.

As used herein, “pharmaceutically acceptable carriers or diluents” are suitable for administration. Certain such carriers enable pharmaceutical compositions to be formulated as, for example, tablets, pills, dragees, capsules, liquids, gels, syrups, slurries, suspension, and lozenges for the oral ingestion by a subject. In certain embodiments, a pharmaceutically acceptable carrier or diluent is sterile water, sterile saline, sterile buffer solution, or sterile artificial cerebrospinal fluid.

As used herein “pharmaceutically acceptable salts” are physiologically and pharmaceutically acceptable salts of compounds. Pharmaceutically acceptable salts retain the desired biological activity of the parent compound and do not impart undesired toxicological effects thereto.

As used herein, an “antisense compound” is a compound capable of achieving at least one antisense activity. In certain embodiments, an antisense compound comprises an antisense oligonucleotide (ASO) and optionally one or more additional features, such as a conjugate group or terminal group.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

For purposes of this disclosure, the entire human PMP22 gene was downloaded from the University of California Santa Cruz genome.ucsc.edu database (SEQ ID NO: 1). Consistent with standard nomenclature, as shown in the Figures, regions of sequence in introns or non-coding portions of the genome appear in lower case letters. Regions of sequence encoding amino acids appear in upper case letters. For the database used and corresponding sequences shown in the Figures, the UCSC database above was used and the option to download the sequences from the Human Assembly December 2013 (GRCh38/hg38) with the protein coding option was selected. If other databases are available and acceptable or become available and acceptable with slight sequence variations, one skilled in the art would understand this description to also cover those variants.

As used herein, the term “complementary” in reference to an oligonucleotide refers to two nucleic acid singles strands or portions of a single strand capable of hybridizing into a double-stranded sequence via hydrogen bonding of complementary bases. Complementary bases include adenine (A) and thymine (T), adenine (A) and uracil (U), cytosine (C) and guanine (G), 5-methyl cytosine (mC) and guanine (G). Complementary oligonucleotides and/or nucleic acids do not need to be complementarity at each positions. Some mismatches can be tolerated. As used herein, “fully complementary” or “100% complementary” in reference to oligonucleotides means that the two oligo-strands have complementary bases at each corresponding position. In certain embodiments, complementary oligonucleotides have only at least 70% complementary bases at each corresponding position.

As used herein, “hybridization,” hybridizing, and the like means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleotides.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “exon skipping” can involve skipping an entire exon or a portion of an exon.

As used herein, an exon-skipping compound” relates to any compound that binds to a pre-mRNA species and induces transcribed mRNA that is stable and measurable, but not full-length (i.e., exon-skipped mRNA).

While a gene or genetic locus may be identified by a particular reference sequence, e.g., the human PMP22 gene, it is understood that the gene corresponding to the reference sequence can comprise various allelic forms or variations in sequence and still be considered by one of ordinary skill in the art to be the same gene.

It is understood that the nucleic acid sequences set forth herein and in each corresponding sequence ID Number (SEQ ID NO) are independent of any modification to a sugar moiety, an inter-nucleoside linkage, or a nucleobase. As such, any nucleic acid of this disclosure, including oligonucleotides, may comprise, independently, one or more modifications to a sugar moiety, an inter-nucleoside linkage, or a nucleobase.

As used herein, a biological sample can be a cell, tissue, organ, or a sample obtained therefrom. For example, a biological sample can be blood, plasma, cerebrospinal fluid (CSF), lymph, skin, saliva, mucus, feces, urine, eye fluid, saliva, stomach fluid, or a sample obtained therefrom.

As used herein, a “biological fluid” is any fluid extracted from an animal, patient, or tissue for analysis. Biological fluids include, but are not limited to, blood, plasma, cerebral spinal fluid, ocular fluid, stomach fluid, saliva, urine, bile, feces, sweat, skin cells, etc. Biological fluids can serve as biological samples. Biological samples can also be obtained from biological fluids.

As used herein, a “negative assay” is an assay where the goal of the assay is to measure a lowering of the total amount of a specific molecule within a sample in the presence of a starting amount of the material that is not zero.

As used herein, unless explicitly stated otherwise, reference to an exon-skipped mRNA and the full-length mRNA is understood to refer to an exon-skipped mRNA and the corresponding full-length mRNA produced from the same pre-mRNA without the induced exon-skipping, even in the absence of the term “corresponding.”

### OVERVIEW

Certain drug approaches have been pursued and published that target diseases where the molecular mode of action is to lower, rather than raise, the total naturally-occurring protein present in a patient by blocking messenger RNA (mRNA) within a patients cells, thus lowering the patients protein levels. Numerous approaches have been described to “silence” the mRNA using anti-sense oligonucleotides, small molecule binding entities, miRNAs, siRNAs, etc.

Often, these molecules fall into a class known as small interfering RNAs (siRNA) (sometimes also known as short interfering RNA or silencing RNA), which are a class of single or double-stranded RNA non-coding RNA molecules, typically 20-27 base pairs in length, similar to miRNA, and operating within the RNA interference (RNAi) pathway. This pathway interferes with the expression of specific genes with complementary nucleotide sequences by degrading mRNA after transcription, preventing translation. Other approaches utilize a gapmer approach to silence RNA by inducing the cleavage of mRNA, and thus degradation of the mRNA to prevent amino acid production. Still other previously described techniques target regulatory regions of the pre-mRNA or mRNA in order to turn off mRNA production or silence the conversion of the mRNA into proteins.

All of these techniques share a “core” limitation, namely they monitor how much RNA goes down—against a biological baseline level—as the drug is added. Thus, they are performing a “negative assay”. It is well understood in the art, for many biological targets, the naturally occurring baseline level of RNA (pre-RNA, mRNA, etc.) can be quite variant based on numerous factors, including but not limited to:
time of day, exercise, and dietary variance amongst subjects studied;
overall health of the research animal or patient;
amount of tissue that is damaged or destroyed resulting in the “leaking-out” of material into the bloodstream; and
degradation of proteins and RNA both in the cells, tissue, and bloodstream.
   (see, e.g., Wegler et al. NAR Genomics and Bioinformatics, 2020, Vol 2, No. 1 (pp 1-11) and Xu and Asakawa, PLOS Computational Biology Jul. 23, 2021 (pp 1-20) for a discussion of the various factors that can effect natural fluctuations of RNA and proteins at baseline levels).

For therapeutic strategies where the goal is to “lower” the amount of functional protein, over dosing a patient or animal can have negative effects on overall health. In many diseases or conditions, the presence of some amount of the protein is required for normal body function and health. Thus, the amount of protein must not be lowered beyond a given desired value or other problems/symptoms may occur. Charcot Marie Tooth Syndrome (CMT) is one example of this situation. For CMT1A, patients have a duplication of the PMP22 protein gene which causes an overproduction of PMP22 mRNA and thus PMP22 protein. However, some amount of PMP22 protein is required for normal patient function. Thus, a strategy to alleviate the symptoms and tissue degradation for CMT1A must find a balance between lowering PMP22 protein a desired amount (to alleviate the issues associated with CMT1A), while still maintaining sufficient amount of functioning PMP22 for normal health. The loss of PMP22 results in a distinct neuropathy called hereditary neuropathy with predisposition to pressure palsy (HNPP). Other problems may also occur such as improper nerve function and potentially death. Another example strategy is to lower the production of proteins through the Nav 1.7 and/or 1.8 pathways to reduce the number of receptors associated with certain pain conditions. Again, it could be problematic to patient health to completely eliminate these proteins and a balance is most preferred in this situation. Another example strategy is to lower the presence of Angiopoietin-like 4 (ANGPTL4) for certain types of cancer and lipid disorders. The presence of ANGPTL4 is critical for other cellular and tissue function, therefore cannot be completely removed.

For most classical drug development, the overall strategy is to study drug effects versus concentration delivered over a large number of cellular and animal experiments to correlate the drugs effect at altering phenotypic responses. Drugs can then be studies in humans (typically at multiple dosing strategies mainly focuses on safety and toxicity profiling) in order to decide upon a single recommended dose across a target patient population. This often leads to drugs that show a positive effect in clinical setting for certain patients, but often leads to showing no measurable phenotypic effect whatsoever in a subset of the patient population due to underdosing or negative side effects due to overdosing for that particular patient. Often, these unresponsive patients are simply being dosed ineffectively based on semi-correlated previous studies that relied on the average response of a large number of patients (or animals in research studies) and the corresponding statistical analysis.

The present disclosure provides a new class of therapeutics that can elicit a molecular response in patients where both the native unaffected mRNA (full-length mRNA) and the “drugged” mRNA (exon-skipped mRNA) can be monitored during the course of treatment and related to downstream protein production. In certain embodiments, this can be done in both the target tissues of interest and/or samples taken from other (e.g., more accessible) portions of the body such as blood plasma, urine, skin samples, cerebral spinal fluid, ocular fluid, etc. This approach allows for careful monitoring of the effect of the drugs proposed both during animal studies and in actual patients during clinical trials and patient deployment.

Provided herein are methods and compositions of selectively down producing functional proteins, without completely blocking production of functional proteins. In some cases, the resulting method produces concatenated proteins that are stable in the cells or body but not fully functioning. In other cases, the concatenated proteins will be less stable than the fully function proteins producing a lower concentration of fully functional protein.

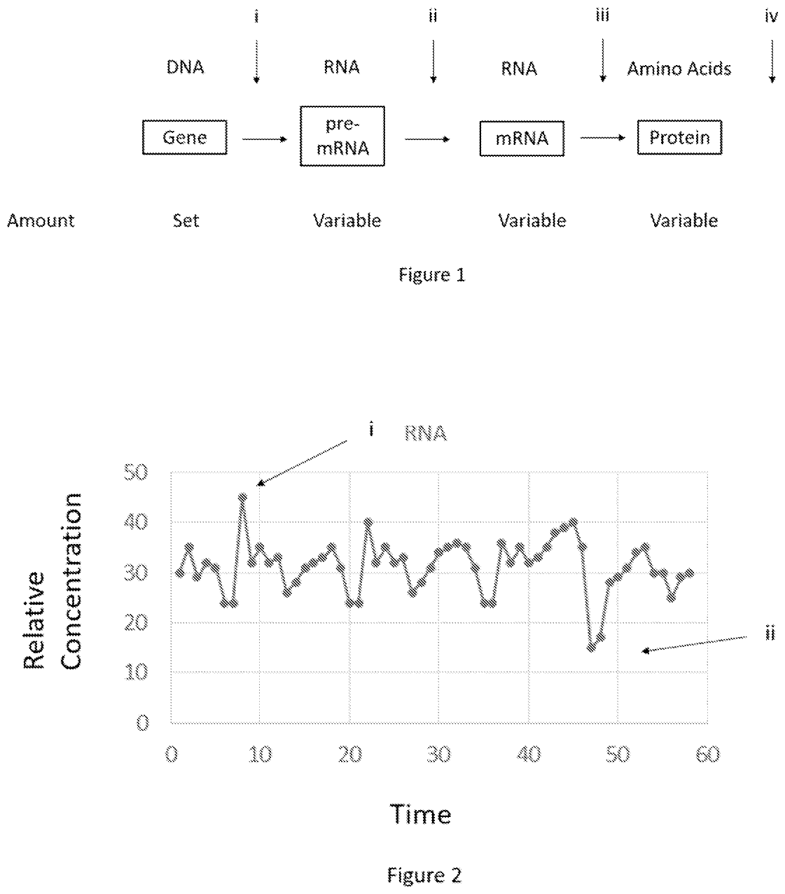

The normal sequence of protein production is shown in FIG. 1. While other processes can be involved in this overall sequence, the simplified version of FIG. 1 is sufficient for this disclosure. Cell nuclei contain copies of genes consisting of DNA. Cellular mechanisms convert the genetic sequences to pre-mRNA that is a copy of the DNA in RNA form and contains all of the intros and exons from the DNA, in normal functioning. The pre-mRNA is further processed by enzymes and co-factors in the cell to create messenger RNA (mRNA). The mRNA contains only the portions of the pre-mRNA referred to as Exons, since the Exons contain the sequences that code for the amino acid sequences that make up the proteins. The introns contained within the pre-mRNA are spliced out during the formation of the mRNA. The mRNA is then converted by still other enzymes and co-factors to create polypeptides (e.g., proteins). In this scheme, the amount of genetic DNA is set for a given cell and does not vary. The amount of pre-mRNA, mRNA, and proteins can vary within a cell and from cell to cell based on a variety of factors and thus is variant over time depending upon the condition of the cell, type of tissue, condition of the animal, age, disease state, etc. A very large number of factors can affect the amount of each of these materials in a cell, tissue, blood, etc. Some of these factors are related to disease, but often the variance of the RNAs and proteins are simply part of normal biological function.

Numerous biochemical techniques have been developed to assess the amount of DNA, RNA, protein, etc. in a biological sample. While certain biochemical techniques (such as amplification of nucleic acids using PCR or other amplification techniques) provide very sensitive means for detecting specific sequences of DNA or RNA, the measurements will always be variable depending on the biological variance of the cells, tissue and animals. Also the methods used to access the materials to measure the DNA, RNA and proteins, including but not limited to sample preparation, handling and storage will vary the measurements. The stability of the biological materials can also have an effect on the ability to measure these materials accurately and reproducibly.

In any situation, no amount of assay development and optimization can overcome the natural variance of protein and nucleic acid samples taken from a subject such as research animal or human including a human patient. This variance is graphically shown in FIG. 2. In FIG. 2, the amount of RNA of a specific sequence is shown over time (Y-axis is relative concentration and X-axis is relative time, in this case we will consider the X-axis to be different days over a 58-day period, but other examples show a similar trend). For example, for the data in FIG. 2, consider the amount of mRNA for a specific gene coding protein in the liver. For each data point, a mouse must be sacrificed, and a portion of the liver materials dissected and prepared for RNA analysis. Thus, in order to generate a single data point for each point in the plot in FIG. 2 would require 58 animals to be studied and sacrificed. For typical biological experiments, more than one animal would be analyzed at each time point, greatly expanding the number of research animals required. In practice, more animals will be used at each time point and fewer time points would be collected in order to analyze a trend in the data. In FIG. 2, the high point (i) and low point (ii) are noted. For a typically functioning healthy mouse, these time points may simply be due to the time of day the sample was collected, exercise that animal performed prior to the measurement, food intake, sleep pattern, etc. Thus, it may be considered that all of the data points within FIG. 2 are considered normal and healthy and the normal level of RNA for these animals would simply be the average and standard deviation of these experiment.

Previous methods for lowering the total amount of functional protein at the molecular level are graphically highlighted in FIG. 1. In this scheme, therapeutics can be added that block to gene to pre-mRNA production (i); block the conversion of pre-mRNA to mRNA (ii); block the mRNA from becoming protein (iii), or degrade, destroy, or partially destroy the mRNA once it is synthesized to block its production to proteins (iii). Other drug strategies can be to apply drugs to affect the functional protein after it is synthesized in the body (iv). The effectiveness of these approaches can be monitored by analyzing the total amount of one of the materials from cells, tissue, or biological fluid, and comparing that amount to the naturally occurring amount present in the absence of the drug.

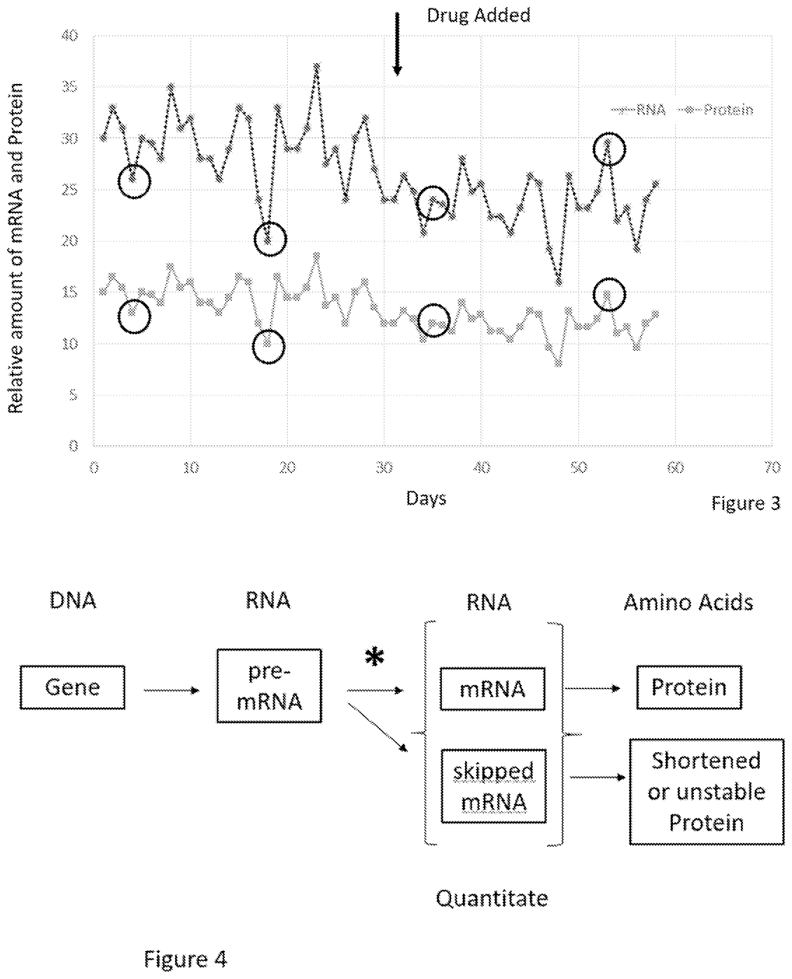

For one example, consider a drug that is meant to block pre-mRNA from forming mRNA by hybridizing or attaching to a key regulatory region and down regulate total mRNA synthesis. Data from such an example is shown in FIG. 3. In this example, the target tissue of interest is extracted from a given laboratory animal (such as a mouse) prior to the dosing of a potential drug and the total mRNA of interest is monitored (for example, using PCR). For the data shown in FIG. 3, many data points have been taken over time allowing for the determination of overall mRNA levels (if sufficient subjects are studied). In FIG. 3, the mRNA levels present over time have been overlayed, as well as the corresponding protein levels (of the protein that is coded by that specific mRNA). Data was collected for 30 time points (in this example 1 time point per day) for 30 days prior to the drug being dosed at day 31. Animals were monitored after the drug was added at each day. As one can see from FIG. 3, if sufficient points were acquired over this period (in this case daily), the average values before and after the drug addition can be calculated and the effect of inhibition calculated (in this case, 20% reduction in mRNA).
mRNA prior to the drug=Average 29.4, Standard Deviation 3.5
mRNA after the addition of the drug=Average 23.6, Standard Deviation 32.8

However, for most experimental situations, it would be extremely problematic or impossible to collect this number of samples in a laboratory setting and even more difficult for actual human patients. The data shown in FIG. 4 would certainly require multiple animals at each time point. A more typical approach is where a number of animals are studied and simply sacrifice for analysis periodically. This is graphically represented in the data from FIG. 4 as the spaced circles, representing time points evenly space over the course of the experiment. Due to biological variance, the timepoint shown in FIG. 4 in circles would actually demonstrate a slight increase in mRNA production, even though at a cellular level, this is not the case.

Also importantly, depending on the tissue type to be targeted by a drug, this type of molecular assessment would be impractical in human patients, since periodic biopsies that would provide sufficient data to observe a trend would neither be practical or ethical.

For demonstration purposes, the protein levels are also shown in FIG. 4 (again, with relative concentration levels). The protein levels could be assessed with numerous other assay techniques such as ELISA binding assays, LCMS, western blots, or other techniques known in the art. In this graph, the proteins levels track (up and down) with the amount of mRNA. However, often protein levels within these tissues will not exactly track with the RNA levels, since proteins often have variety different stability levels and time factors as compared to RNA. That is, even if the RNA levels go down on a given day or time point, it may require a much longer period of time to observe the corresponding protein levels to change.

Thus, this example of a therapeutic approach is limited by major factors including: fairly large changes in total mRNA must be present in the animal to notice a statistically meaningful difference; even for large changes, large numbers of subjects and samples must be collected and tested to determine and overall statistically change and changes in a single given subject is very problematic if accurate total mRNA blocking is desired; and for treatment in a single subject, both sample collection (which may be impractical) and natural variance of the subject makes the monitoring of how the drug is effecting a patients mRNA specifically over time very problematic or impossible.

It is possible to extract biological fluid (such as blood in this example) on a more regular basis an analyze any RNA or protein within that fluid without sacrificing the animal or patient. However, the level of RNA and protein changes within the blood will have even greater variance based on a number of factors than in the target tissue and cells and thus will be practically meaningless when trying to correlate to actual drug activity, in cases where you are trying to lower the amount of total protein within completely eliminating it.

Provided herein are compounds and methods for eliciting the production of an exon-skipped mRNA that is different than the full-length mRNA and above naturally occurring levels. The exon-skipped mRNA induced in embodiments of this disclosure is not present or present only at very low levels in naturally occurring tissue, thus provided for a “positive assay” mode where the baseline levels of the exon-skipped mRNA is zero or near zero, and the effect of the drug is determined with the positive increase in the signal associated level of the mRNA.

During normal mRNA production, mistakes will occur in the conversion of pre-mRNA to mRNA. Thus, there may be a very small level of non-full-length mRNA present in certain biological samples, but it will have little or no effect on biological function. Often these “mistakes” are undetectable or detectable only at extremely low levels. Thus, this disclosure relates to situations where the exon-skipped mRNA produced by the drug is >10% of the total mRNA level present in a given sample as compared to the normal full-length mRNA. The amount of the exon-skipped mRNA will correlate (but not necessarily in a linear fashion) with a reduction of full-length functional proteins. This disclosure relates to the application of drugs that induce an exon skipping event, the compounds themselves, and the measurement of the exon-skipped mRNA within a sample to determine the effectiveness and activity of the therapeutic used at lowering the total target functional protein level.

In certain embodiments, exon-skipped mRNAs can be measured in the subject's tissue, blood, skin, etc., to determine the activity of the compound. The presence of the exon-skipped mRNAs can be monitored over time (in between dosing of said compounds) to determine the long-term effect of the compounds on protein production. The presence of the exon-skipped mRNAs can be monitored over time (in between dosing of said compounds) to determine when additional compound should be administered to the subject to increase the level of exon-skipped mRNAs and thus further lower the production of the targeted protein.

In certain embodiments, the presence of the exon-skipped mRNAs are monitored and quantitated. In certain embodiments, the measurement of the exon skipped mRNA alone can be correlated to total full-length protein production. In certain embodiments, the term “full-length” protein is used to refer to functional protein. In other embodiments, the amount of the full-length mRNA is also measured and quantitated and is compared to the amount of exon-skipped mRNAs present to determine a ratio value. The ratio of the two mRNAs will correlate to down production of full-length protein, and thus therapeutic activity. Numerous mathematical methods can be used to correlated both the amount of exon-skipped mRNA and full-length mRNA to full-length protein production.

In certain embodiments, the exon-skipped mRNAs produced are stable or somewhat stable and can be analyzed (outside of the tissue material or animal) by standard nucleic acid detection methods. In other embodiments, the exon-skipped mRNAs degrade into secondary exon-skipped mRNAs that can also be monitored to determine the overall protein level effects, so long as these secondary exon skipped mRNAs increase in concentration after the addition of the drug by greater that 1% above naturally occurring levels. In certain embodiments, the ratio of the secondary exon-skipped mRNAs are compared to the full-length mRNA. In other embodiments, at least one, two, three, four, five, or all of the exon-skipped mRNAs are measured to determine the overall effect of the protein levels.

In certain embodiments, exon-skipped mRNAs that are to be measured are sequences of RNA that do not overlap with other “naturally occurring” RNA sequences present in the body, e.g., coming from other genes or RNA processes that may be present and produce spurious background signals to the assays that are not related to the compounds effect on protein production.

In certain embodiments, the compounds are dosed to elicit a 50% (+/−10%) reduction of full-length mRNA. In other embodiments, the desired reduction of protein for a target tissue is higher than 50%, while in other applications, it is lower that 50%. In other embodiment, the compounds are dosed to achieve a reduction in full-length mRNA between 10-90% (+/−5%). In still other embodiments, the compounds are dosed to achieve a reduction in full-length mRNA between 1-99% (+/−0.9%). Measurements of exon-skipped mRNAs can be monitored over time and compound re-administered to produce the desired level of exon-skipped mRNAs.

In certain embodiment, the exon-skipped mRNAs are “semi-stable” over the course of the sample collection and monitoring. In these examples, the ratio of exon-skipped mRNAs to full-length mRNA may not appear as 1:1 in the full assay due to the partial degradation. However, the desired ratio of the measurement of the exon-skipped mRNAs (or simply the total Exon-skipped mRNAs present) can be correlated to disease progression, full-length mRNA, protein levels, secondary biomarkers, etc during larger studies and used as a benchmark for actual patient testing. For example, it may be found during research studies that 80% of the exon-skipped mRNA degrades quickly after sample collection but 20% remains stable. Thus, if the measured exon-skipped mRNA a few days later appears as 0.2 picomoles and the full-length mRNA is 1.0 picomole, the actual presence of the exon-skipped mRNA at the cellular level is 1.0 picomoles and the effect on the protein production is actually a 50% reduction.

An overall schematic of the present disclosure in shown in FIG. 4. A therapeutic compound (*) is added to the animal, tissue, cell, etc. that forces a change in the conversion of pre-mRNA to mRNA and creates a stable, measurable shortened mRNA product (exon-skipped mRNA) that will produce a shortened, non-functional, and/or unstable protein as the product rather than full-length functional protein.

In FIG. 4, the amount of full-length mRNA and the amount of exon-skipped mRNA can both be quantitated and compared to determine the cellular activity. As mentioned, the full-length mRNA will go up and down over time and at various normal conditions. In certain cases, the amount of exon-skipped mRNA may vary over time as well when the therapeutic is present. In certain embodiments, both the full-length mRNA and the exon-skipped mRNA are quantitated and compared. This comparison is used to quantitate the effectiveness of the therapeutic rather than the total amount of either product individually. This comparison can be normalized to the biological variance that will occur within the cell, tissue or animal and be dependent more fully on the effectiveness of the drug used. The comparison can be a ratio of the full-length to the exon skipped, the difference between the two, or any other mathematical comparison.

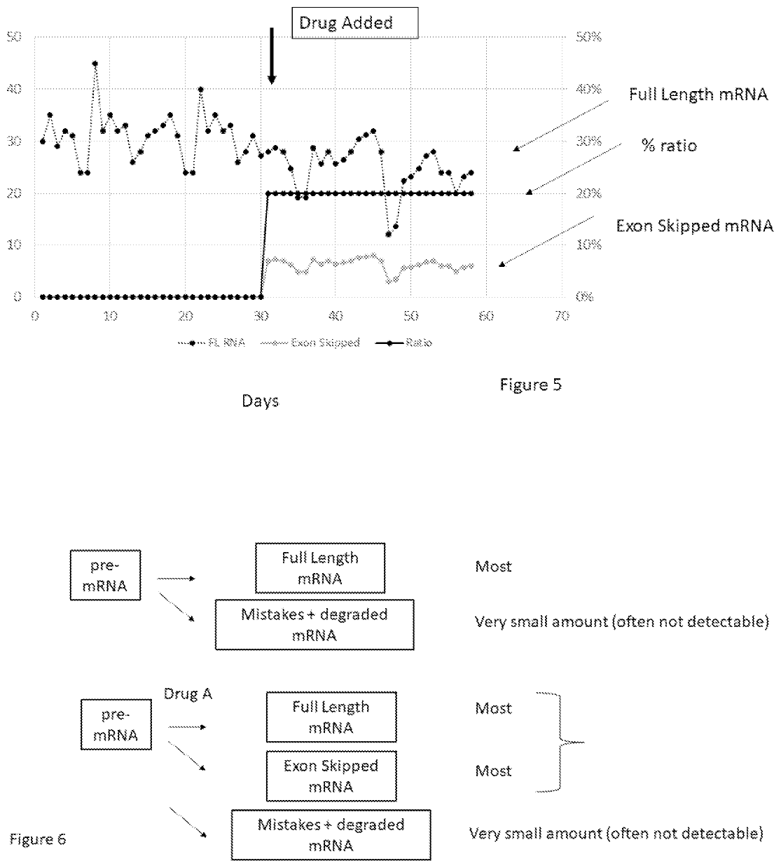

Graphically, this analysis is shown in FIG. 5. In this example the full-length mRNA is shown over time and varies significantly (due to general variation) prior to the addition of the drug at day 30. Prior to the addition of the drug there is no exon-skipped mRNA in the patient, animal, tissue etc. (or the level is <1% of the total value). After the drug is added, the level of exon-skipped mRNA rises to a level corresponding to the activity of the drug and the total level of pre-mRNA in the cell at any given time. Thus, it also varies after day 30 as the parent pre-mRNA varies (the amount of exon skipped mRNA is related to both the amount of drug present and the amount of full-length pre-mRNA present and the effectiveness of the drug at that concentration). However, the ratio of the two amounts will vary only based on the activity of the drug after day 30 (plus or minus the variance of the measurements). Shown in FIG. 5 is the ratio of the full-length/exon-skipped mRNA over time (labeled % ratio and for this example calculated as the amount of exon-skipped mRNA/full-length mRNA, but other mathematical ratios can be utilized). In this example, enough drug was added to convert 20% of the full-length mRNA to exon-skipped mRNA; thus the ratio is 20% after addition when the ratio is plotted. The relative amount of the RNA present is labeled on the left Y axis and % ratio on the right Y axis.

As mentioned, the normal conversion of pre-mRNA to full-length mRNA can produce non-full-length mRNA during normal biological processes (see FIG. 6). In this figure, these non-full-length mRNAs are shown as mistakes and degradants and can, in certain situations, include exon-skipped versions of the full-length mRNA. However, even though these exon-skipped versions of the mRNA are typically at very low concentrations, they must be considered. Thus, the present disclosure relates to drug compounds and methods as shown in the bottom half of FIG. 6 where the exon-skipped mRNA caused by the addition of the drug (labeled Drug A in FIG. 6) produces an amount of exon-skipped mRNA that is measurable above any naturally-occurring mistakes in the mRNA production. In certain embodiments, the amount of exon-skipped mRNA produced by the drug added is greater than about 1% of the full-length mRNA. In certain embodiments, the amount of exon-skipped mRNA produced by the drug added is greater than about 2%, 3%, 4%, 5%, 6%, 7%, 8%, 9%, 10%, 15%, 20%, 25%, 30%, 40%, 50%, 60%, 75%, 80%, 90%, or 99% of the full-length mRNA. In certain embodiments, the amount of exon-skipped mRNA produced by the drug added is between and of about 2%, 3%, 4%, 5%, 6%, 7%, 8%, 9%, 10%, 15%, 20%, 25%, 30%, 40%, 50%, 60%, or 75% and any of about 3%, 4%, 5%, 6%, 7%, 8%, 9%, 10%, 15%, 20%, 25%, 30%, 40%, 50%, 60%, 75%, 80%, 90%, 99%, or 100% of the full-length mRNA. In some embodiment, the amount of exon-skipped mRNA generated by the addition of the drug is greater than 10% larger than any naturally-occurring skipped mRNA that is present in the tissue sample of interest and of the identical sequence produced by the exon skipping drug.

Numerous types of drugs are envisioned herein, so long as they induce and exon-skipping event in the synthesis of mRNA for a target gene. Anti-sense oligonucleotides are described that bind to a portion of the pre-mRNA to induce a skipping event. Small molecule drugs can also be utilized that specifically bind to the intron-exon regions of the exon to be skipped during conversion to mRNA. These molecules can bind to the 3D structure that is formed by the pre-mRNA prior to intro removal. Antibodies that re specific to a given intro-exon region can also be used. Other selective enzymes that specifically target a portion of a genetic pre-mRNA can also be used, such as CRISPR.

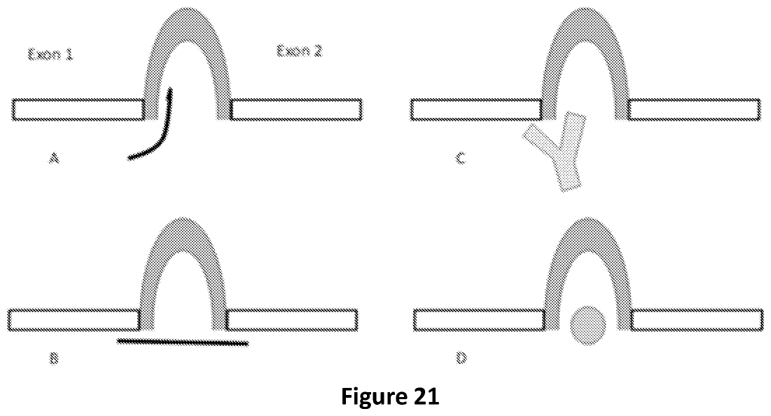

The present disclosure provides for a composition comprising a compound that specifically targets a pre-mRNA to induce production of an exon-skipped mRNA via exon-skipping. The compound can be any type of compound that induces exon-skipping, as exon-skipping is described in detail elsewhere herein. Examples of exon-skipping compounds include, but are not limited to, nucleic acids such as antisense oligonucleotides (ASOs) (e.g., FIGS. 21A and 21C), antibodies (FIG. 21C), and small molecules (FIG. 21D) that can bind to the exon/intron junction region, or in the vicinity thereof, of a pre-mRNA.

In certain embodiments, the exon-skipped mRNA produced is detectable and in certain embodiments, the exon-skipped mRNA is measurable above a background level. That is, due to naturally-occurring aberrant pre-mRNA processing, there may naturally be a low background level of the exon-skipped mRNA produced. Addition of the exon-skipping compound induces increased production of the exon-skipped mRNA and the amount of additionally produced exon-skipped mRNA is measurable above the background level. In certain embodiments, the exon-skipped mRNA is a non-naturally occurring mRNA that is not produced at all in the cell absent addition of the exon-skipping compound. Thus, the background level is zero and the exon-skipped mRNA is measurable above the background level. In certain embodiments, the compound reduces, but does not completely abolish, the amount of full-length mRNA expressed in a cell. For reasons that are discussed in more detail elsewhere herein, for certain purposes it may not be desirable or may even be detrimental, for example to a patient being treated with a exon-skipping compound, to completely abolish production of the full-length mRNA and/or its encoded protein product.

In certain embodiments, the exon-skipped mRNA encodes a non-stable and/or non-functional protein product. For example, a non-functional protein product can be produced when the codons downstream of the skipped exon are out of frame in comparison to the full-length mRNA. The out of frame sequence can lead to the portion of the protein encoded downstream of the skipped exon to be translated differently (i.e., resulting in a different amino acid sequence) and/or leading to an earlier stop codon and termination. Such a protein may also be unstable and/or recognized as aberrant and degraded more quickly. In another embodiment, a non-stable and/or non-functional protein product can be produced even though the codons downstream of the skipped exon remain in frame in comparison to the full-length mRNA, e.g., wherein a portion of the protein product corresponding to the sequence of the skipped exon is missing.

In certain embodiments, the compound that specifically targets the pre-mRNA is an antisense oligonucleotide (ASO). In certain embodiments, the ASO is a PMO as described elsewhere herein (e.g., in certain of the Examples that follow). In certain embodiments, at least one of the sugars in the nucleic acid backbone of the ASO is 2′-OMe-substituted.

Various methods of increasing the uptake of nucleic acids into cells are known to those of ordinary skill in the art. Thus, for example, in certain embodiments an ASO is conjugated to a delivery molecule to enhance cellular uptake. In certain embodiments, the delivery molecule enhances uptake by a specific cell type greater than other cell types. Illustrative examples of delivery molecules include antibodies, peptides, lipids, and small molecules. In certain embodiments, the ASO is formulated into a nano-particle to enhance uptake.

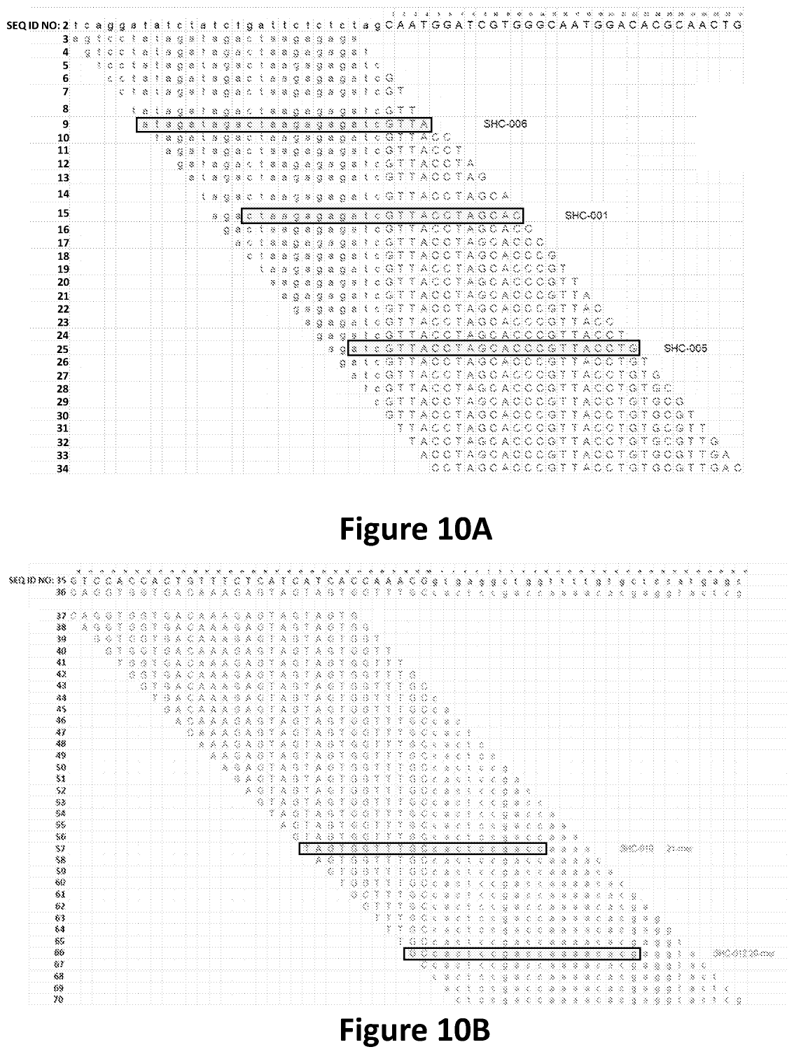

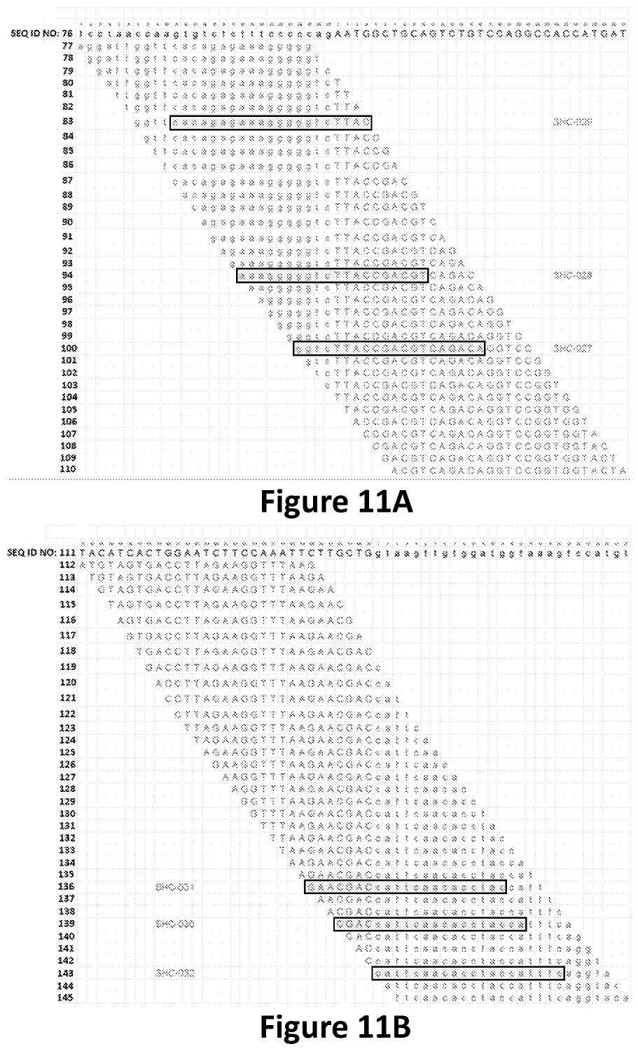

As shown in, e.g., FIG. 10A,B and FIG. 11A,B, an ASO of this disclosure comprises or consists of a complementary region to a target region of the pre-mRNA. One of ordinary skill in the art will recognize that the complementary region does not need to be 100% complementary to the target region as long as it possesses enough complementarity to hybridize to the target region. In certain embodiments, the ASO comprises or consists of a complementary region that is at least 70%, 75%, 80%, 85%, 90%, 95%, 96%, 97%, 98%, 99%, or 100% complementary to a target region of the pre-mRNA. For ASOs up to about 30, 40, or 50 nucleotides in length, the ASO comprises or consists of a complementary region that is complementary to a target region of the pre-mRNA except for one, two, three, four, or five mismatches.

As discussed in detail elsewhere herein, it has been shown that exon skipping can be induced by targeting intron/exon junctions of a pre-mRNA. Thus in certain embodiments, the target region of the pre-mRNA spans an intron/exon junction of one of the coding exons. In certain embodiments, the target region of the pre-mRNA spanning an intron/exon junction comprises at least 2, 3, 4, 5, 6, 7, 8, 9, 10, 11, or 12 nucleotides of the intron and a portion of the exon. In certain embodiments, the target region of the pre-mRNA consists of 2, 3, 4, 5, 6, 7, 8, 9, 10, 11, or 12 nucleotides of the intron and a portion of the exon. In certain embodiments, the target region of the pre-mRNA spanning an intron/exon junction comprises at least 2, 3, 4, 5, 6, 7, 8, 9, 10, 11, or 12 nucleotides of the exon and a portion of the intron. In certain embodiments, the target region of the pre-RNA consists of 2, 3, 4, 5, 6, 7, 8, 9, 10, 11, or 12 nucleotides of the exon and a portion of the intron. In certain embodiments, binding in a cell of the complementary region of the ASO to the target region of the pre-mRNA results in exon skipping of one or more exons during RNA transcription. In certain embodiments, it induces production of an exon-skipped mRNA. In certain embodiments, it results in a decrease of the corresponding full-length mRNA from the pre-mRNA.

Certain compounds and compositions of this disclosure are contemplated for use in medical treatment such as of a disease or medical condition. In certain embodiments, the target pre-mRNA is associated with a disease. For example, the disease can be a genetic disorder such as CMT or Huntington's disease. The disease, however, doesn't need to be a genetic disorder, for example, acid reflux disease. In certain embodiments, the disease may or may not have an inherited genetic cause, such as cancer. In certain embodiments, the medical treatment may even be of a non-disease nature such as inhibiting pain receptors. In certain embodiments, the target pre-mRNA is associated with a disease because produces too much mRNA and/or protein product, for example because the gene encoding the pre-mRNA is too highly expressed or excess copies of the gene are present in the genome. In certain embodiments, the composition is a therapeutic composition comprising a pharmaceutically acceptable carrier or diluent which are well-known to those of ordinary skill in pharmaceutical formulation. In certain embodiments, the compound is an ASO and the ASO is administered as a pharmaceutically acceptable salt.

One illustrative example of this disclosure provides for a composition comprising an antisense oligonucleotide (ASO) that comprises or consists of a complementary region that is complementary, or complementary except for one, two, three, four, or five mismatched nucleotides, to a target region of a PMP22 pre-mRNA. In certain embodiments, the complementary region is at least 70%, 75%, 80%, 85%, 90%, 95%, 98%, 99%, or 100% complementary to a target region of the pre-mRNA. It is understood for any embodiment disclosed herein that the ASO need not be complementary to the full length of the target region but is complementary to a sufficient portion of the target region to hybridize, i.e., the defined target region can be longer than the ASO complementary region and longer than the entire ASO. In certain embodiments, the complementary region of the ASO is complementary to a portion or subset/fragment of the target region sequence. In certain embodiments, the complementary region and the target region are the same length. As described in greater detail elsewhere herein, binding in a cell of the complementary region of the ASO to the target region of the PMP22 pre-mRNA induces exon skipping during RNA transcription. Binding of the ASO to the target region can reduce full-length PMP22 mRNA production. It can also lead to the production of exon-skipped PMP22 mRNA. Binding of the ASO to the target region can lead to a reduction in functional PMP22 protein and/or production of non-functional PMP22 protein. For any embodiments disclosed herein, not limited to just PMP22, both the reduction in full-length mRNA and the production of exon-skipped mRNA can be detected and measured. The reduction in functional protein and/or the production of non-functional protein can also be detected and measured. Further the correlation and/or ratio between full-length and exon-skipped mRNAs and functional and non-functional proteins can be determined and calculated for purposes such as disclosed in detail elsewhere herein.

In certain embodiments, the ASO comprises or consists of a complementary region of at least about 12, 13, 14, 15, 16, 17, 18, 19, 20, 21, 22, 23, or 24 contiguous nucleotides that are complementary, or complementary except for one, two, three, four, or five mismatched nucleotides, to the PMP22 pre-mRNA target region. One of ordinary skill in the art would recognize that the length of the ASO's complementary region can vary depending on, for example, the PMP22 pre-mRNA target region sequence and/or the particular application or conditions of administration. Illustrative examples of ASOs with a complementary region of “contiguous nucleotides” are disclosed in the Examples that follow (e.g., FIG. 10A,B and FIG. 11A,B).

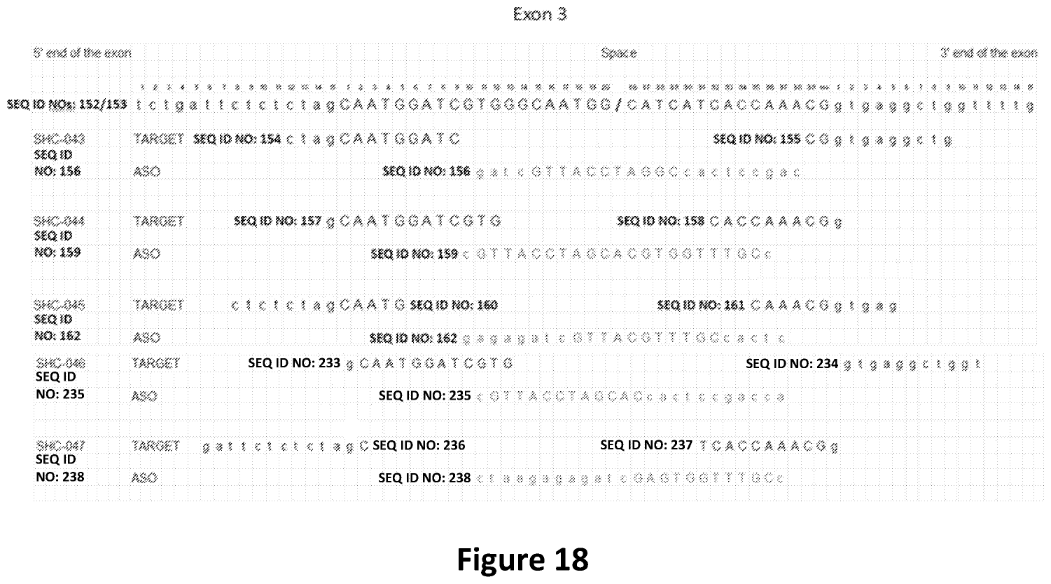

In certain embodiments, the PMP22 pre-mRNA target region comprises two separate segments of the PMP22 pre-mRNA such as described in Example 6 (FIG. 18). In certain embodiments, the ASO comprises a complementary region of at least about 6, 8, 9, 10, 11, or 12 nucleotides that are complementary, or complementary except for one, two, or three mismatched nucleotides, to a first segment of contiguous sequence of the PMP22 pre-mRNA target region and the ASO also comprises a complementary region of at least about 6, 8, 9, 10, 11, or 12 nucleotides that are complementary, or complementary except for one, two, or three mismatched nucleotides, to a second segment of contiguous sequence of the PMP22 pre-mRNA target region. Thus, the complementary region of the ASO hybridizes to the first and second segments of the PMP22 pre-mRNA and also spans a region of the PMP22 pre-mRNA that it is not complementary/does not hybridize to.

In certain embodiments of any ASO of this disclosure, whether the pre-mRNA target region is one contiguous segment or two separate segments, the ASO comprises or consists of a complementary region between any of about 12, 13, 14, 15, 16, 17, 18, 19, 20, 21, 22, 23, 24, 25, 30, 35, 40, or 45 and any of about 13, 14, 15, 16, 17, 18, 19, 20, 21, 22, 23, 24, 25, 26, 27, 28, 29, 30, 35, 40, 45, or 50 nucleotides that are complementary, or complementary except for one, two, three, four, or five mismatched nucleotides, to the pre-mRNA target region. In certain embodiments, the ASO comprises or consists of a complementary region of 16, 17, 18, 19, 20, 21, 22, 23, 24, or 25 nucleotides that are complementary, or complementary except for one, two, three, four, or five mismatched nucleotides, to the pre-mRNA target region.

In certain embodiments of any ASO of this disclosure, whether the pre-mRNA target region is one contiguous segment or two separate segments, the ASO has a length of any of about 8, 9, 10, 11, 12, 13, 14, 15, 16, 17, 18, 19, 20, 21, 22, 23, 24, 25, 30, 35, 40, 45, 50, 60, or 75 and any of about 13, 14, 15, 16, 17, 18, 19, 20, 21, 22, 23, 24, 25, 26, 27, 28, 29, 30, 35, 40, 45, 50, 60, 75, or 100 nucleotides. In certain embodiments, an ASO is between any of about 8, 9, 10, 11, 12, 13, 14, 15, 16, 17, 18, 19, 20, 21, 22, 23, 24, 25, 26, 27, 28, or 29 and any of about 9, 10, 11, 12, 13, 14, 15, 16, 17, 18, 19, 20, 21, 22, 23, 24, 25, 26, 27, 28, 29, or 30 nucleotides long. In certain embodiments, an ASO is between about 12 and 30 nucleotides long. In certain embodiments, an ASO is between about 15 and 25 nucleotides long. In certain embodiments, an ASO is between about 18 and 25 nucleotides long.

In certain embodiments, the ASO is a modified and/or synthetic oligonucleotide as known in the art and/or defined elsewhere herein. For example, the ASO can be a phosphorodiamidate morpholino oligomer (PMO).

Although the ASO causes PMP22 pre-mRNA exon skipping, in certain embodiments this can be done in a manner in which downstream exons are still expressed as they would be from full-length PMP22 pre-mRNA, except for absent the portion from the skipped exon. In certain embodiments, however, the exon skipping forces early termination of protein translation and/or downstream exons to be out of frame.

It has been discovered that exon skipping and the production of exon-skipped PMP22 mRNA has certain advantages over prior methods that do not disclose producing exon-skipped PMP22 mRNA. Thus, in certain embodiments, the target region of the PMP22 pre-mRNA spans an intron/exon junction of at least one of the coding exons (e.g., PMP22 Exon 2, Exon 3, Exon 4, and Exon 5). The targeted intron/exon junction can be at the 3′-end and/or the 5′-end of an exon. For example, in certain embodiments, the target region of the PMP22 pre-mRNA comprises the 3′-end of an exon. And, in certain embodiments the target region of the PMP22 pre-mRNA comprises the 5′-end of an exon. In certain embodiments, the target region of the PMP22 pre-mRNA spans an intron/exon junction comprising at least 2, 3, 4, 5, 6, 7, 8, 9, 10, 11, or 12 nucleotides of the intron and a portion of the exon (e.g., FIG. 10A,B and FIG. 11A,B). In certain embodiments, the target region of the PMP22 pre-mRNA consists of 2, 3, 4, 5, 6, 7, 8, 9, 10, 11, or 12 nucleotides of the intron and a portion of the exon. Likewise, in certain embodiments, the target region of the PMP22 pre-mRNA spans an intron/exon junction comprising at least 2, 3, 4, 5, 6, 7, 8, 9, 10, 11, or 12 nucleotides of the exon and a portion of the intron (e.g., FIG. 10A,B and FIG. 11A,B). In certain embodiments, the target region of the PMP22 pre-mRNA consists of 2, 3, 4, 5, 6, 7, 8, 9, 10, 11, or 12 nucleotides of the exon and a portion of the intron. In certain embodiments, the exon portion of the intron/exon junction comprises PMP22 Exon 3 (FIG. 10A,B). In certain embodiments, the exon portion of the intron/exon junction comprises PMP22 Exon 4 (FIG. 11A,B).

In certain embodiments, the PMP22 pre-mRNA target region comprises the 5′-end of Exon 3. For example, wherein the PMP22 pre-mRNA target region comprises or consists of SEQ ID NO: 2 or a portion or subset/fragment thereof.

In certain embodiments, the PMP22 pre-mRNA target region comprises the 3′-end of Exon 3. For example, wherein the PMP22 pre-mRNA target region comprises or consists of SEQ ID NO: 35 or a portion or subset/fragment thereof.

In certain embodiments, the PMP22 pre-mRNA target region comprises the 5′-end of Exon 4. For example, wherein the PMP22 pre-mRNA target region comprises or consists of SEQ ID NO: 76 or a portion or subset/fragment thereof.

In certain embodiments, the PMP22 pre-mRNA target region comprises the 3′-end of Exon 4. For example, wherein the PMP22 pre-mRNA target region comprises or consists of SEQ ID NO: 111 or a portion or subset/fragment thereof.

In certain embodiments, the PMP22 pre-mRNA target region comprises the 5′-end of Exon 2. For example, wherein the PMP22 pre-mRNA target region comprises or consists of SEQ ID NO: 163 or a portion or subset/fragment thereof.

In certain embodiments, the PMP22 pre-mRNA target region comprises the 3′-end of Exon 2. For example, wherein the PMP22 pre-mRNA target region comprises or consists of SEQ ID NO: 198 or a portion or subset/fragment thereof.

In certain embodiments, the ASO comprises or consists of a complementary region that is complementary, or complementary except for one, two, three, four, or five mismatched nucleotides, to SEQ ID NO: 2 (Exon 3, 5′-end), SEQ ID NO: 35 (Exon 3. 3′-end), SEQ ID NO: 76 (Exon 4, 5′-end), SEQ ID NO: 111 (Exon 4, 3′-end), SEQ ID NO: 163 (Exon 2, 5′-end), and/or SEQ ID NO: 198 (Exon 2, 3′-end), or a subset/fragment thereof sufficient to hybridize to PMP22 pre-mRNA. In certain embodiments, the ASO comprises or consists of a nucleotide sequence of SEQ ID NOs: 3-34 (Exon 3, 5′-end), SEQ ID NOs: 37-70 (Exon 3, 3′-end), SEQ ID NOs: 77-110 (Exon 4, 5′-end), SEQ TD NOs: 112-145 (Exon 4, 3′-end), SEQ ID NOs: 163-196 (Exon 2, 5′-end), or SEQ ID NOs: 198-231 (Exon 2, 3′-end), or a subset/fragment thereof sufficient to hybridize to PMP22 pre-mRNA. In certain embodiments, the ASO comprises or consists of a nucleotide sequence of SEQ ID NOs: 3-34 (Exon 3, 5′-end), SEQ ID NOs: 37-70 (Exon 3, 3′-end), SEQ ID NOs: 77-110 (Exon 4, 5′-end), SEQ ID NOs: 112-145 (Exon 4, 3′-end), SEQ ID NOs: 165-197 (Exon 2, 5′-end), or SEQ ID NOs: 199-232 (Exon 2, 3′-end), except for having one, two, or three nucleotide substitutions, or a subset/fragment thereof sufficient to hybridize to PMP22 pre-mRNA.

In certain embodiments, the ASO comprises or consists of the nucleic acid sequence of: SEQ ID NO: 71 (SHC-006 25-mer), SEQ ID NO: 72 (SHC-001 24-mer), SEQ ID NO: 73 (SHC-005 25-mer), SEQ ID NO: 74 (SHC-010 21-mer), SEQ ID NO: 75 (SHC-012 20-mer), SEQ ID NO: 146 (SHC-029 21-mer), SEQ ID NO: 147 (SHC-028 20-mer), SEQ ID NO: 148 (SHC-027 20-mer), SEQ ID NO: 149 (SHC-031 21-mer), SEQ ID NO: 150 (SHC-030 20-mer), SEQ ID NO: 151 (SHC-032 20-mer), SEQ ID NO: 156, SEQ ID NO: 159, SEQ ID NO: 162, SEQ ID NO: 235, or SEQ ID NO: 238. In certain embodiments, the ASO comprises or consists of the nucleic acid sequence of: SEQ ID NO: 71 (SHC-006 25-mer), SEQ ID NO: 72 (SHC-001 24-mer), SEQ ID NO: 73 (SHC-005 25-mer), SEQ ID NO: 74 (SHC-010 21-mer), SEQ ID NO: 75 (SHC-012 20-mer), SEQ ID NO: 146 (SHC-029 21-mer), SEQ ID NO: 147 (SHC-028 20-mer), SEQ ID NO: 148 (SHC-027 20-mer), SEQ ID NO: 149 (SHC-031 21-mer), SEQ ID NO: 150 (SHC-030 20-mer), SEQ ID NO: 151 (SHC-032 20-mer), SEQ ID NO: 156, SEQ ID NO: 159, SEQ ID NO: 162, SEQ ID NO: 235, or SEQ ID NO: 238, except for having one, two, or three nucleotide substitutions.

Provided for herein is a method of measuring the amount of an exon-skipped mRNA produced in response to an exon-skipping inducing compound. In certain embodiments, the method includes measuring the amount of the exon-skipped mRNA produced against an internal control. In certain embodiments, the method comprises:
(i) administering a composition of this disclosure to a cell to induce exon-skipping of a target pre-mRNA and production of the exon-skipped mRNA;
(ii) obtaining a sample comprising the exon-skipped mRNA; and
(iii) measuring in the sample the amount of the exon-skipped mRNA.

In certain embodiments, the amount of exon-skipped mRNA produced is at least about 1%, 2%, 3% 4%, 5%, 6%, 7%, 8%, 9%, 10%, 15%, 20%, 25%, 30%, 40%, or 50% above a baseline amount of the exon-skipped mRNA in an untreated control.

Certain embodiments further comprise (iv) measuring the amount of the corresponding full-length mRNA in the sample and comparing the amount of the exon-skipped mRNA to the amount of corresponding full-length mRNA. In certain embodiments, the exon-skipping inducing compound reduces, but does not completely abolish, the amount of full-length mRNA expressed from the target pre-mRNA such that the sample obtained comprises both the exon-skipped mRNA and the full-length mRNA and the amount of the full-length mRNA can be measured and compared to the amount of the exon-skipped mRNA. In certain embodiments, the sample comprises the cell but in other embodiments, the amount of the mRNAs can be measured free of cells and correlated to the amount of cells. In certain embodiments, the amount of exon-skipped mRNA produced by the compound added is greater than about 2%, 3%, 4%, 5%, 6%, 7%, 8%, 9%, 10%, 15%, 20%, 25%, 30%, 40%, 50%, 60%, 75%, 80%, 90%, or 99% of corresponding the full-length mRNA. In certain embodiments, the amount of exon-skipped mRNA produced by the compound added is between and of about 2%, 3%, 4%, 5%, 6%, 7%, 8%, 9%, 10%, 15%, 20%, 25%, 30%, 40%, 50%, 60%, or 75% and any of about 3%, 4%, 5%, 6%, 7%, 8%, 9%, 10%, 15%, 20%, 25%, 30%, 40%, 50%, 60%, 75%, 80%, 90%, 99%, or 100% of the corresponding full-length mRNA. In certain embodiments, the reduction in the amount of the full-length mRNA is not more than about 90%, 80%, 70%, 60%, 50%, 40%, 30%, 20%, or 10%. In certain embodiments, the reduction in the amount of full-length mRNA is between any of about 90%, 80%, 70%, 60%, 50%, 40%, 30%, or 20% to any of about 80%, 70%, 60%, 50%, 40%, 30%, 20%, or 10%. In certain embodiments, the amount of full-length protein encoded by the full-length mRNA is reduced. In certain embodiments, the reduction in the amount of the full-length protein is not more than about 90%, 80%, 70%, 60%, 50%, 40%, 30%, 20%, or 10%. In certain embodiments, the reduction in the amount of full-length protein is between any of about 90%, 80%, 70%, 60%, 50%, 40%, 30%, or 20% to any of about 80%, 70%, 60%, 50%, 40%, 30%, 20%, or 10%.

As used anywhere herein, “administering to the cell” is understood to cover all situations where the ASO is placed in contact with the cell in a manner that the cell may take-up the ASO so that the ASO may exert its antisense activity. For example, administering to the cell includes exposing cells in an in vitro experiment to the ASO, such as to cells grown in tissue culture. Administering to the cell also includes providing the ASO to a subject, such as a research animal in an in vivo experiment, such that at least one cell of the subject, through administration locally, systemically, etc., is contacted with the ASO. Administering to the cell also includes providing the ASO to a patient, such as treating a human patient, such that at least one cell of the patient, through administration locally, systemically, etc., is contacted with the ASO. Thus, the subject cell may reside in a tissue, organ, body part, biological fluid, whole organism, and the like.

In certain embodiments the cell is in a subject such as a research animal or human patient, the composition is administered to said subject, and the sample obtained is a biological sample from said subject.

Provided herein is a method of adjusting the dosing of an exon-skipping inducing compound. Adjusting the dosing may be desired, for example, to optimize the efficacy of a treatment and/or to prevent or reduce side effects. In certain embodiments, the method comprises:
(i) administering a dose of a composition of this disclosure to a cell to induce exon-skipping of a target pre-mRNA and production of an exon-skipped mRNA therefrom. In certain embodiments, the compound reduces, but does not completely abolish, the amount of full-length mRNA expressed from the target pre-mRNA;
(ii) obtaining a sample comprising the exon-skipped mRNA and the corresponding full-length mRNA;
(iii) measuring in the sample the amount of the exon-skipped mRNA and the amount of the corresponding full-length mRNA;
(iv) determining the ratio between the amount of exon-skipped mRNA and the amount of corresponding full-length mRNA; and
(v) adjusting the dosing of the composition to be administered based on the ratio between the amount of exon-skipped mRNA and the amount of corresponding full-length mRNA. For example, wherein the amount of the dosing can be adjusted and/or the frequency of administration of the dose can be adjusted.

In certain embodiments, the sample comprises the cell but in other embodiments, the amount of the mRNAs can be measured free of cells and correlated to the amount of cells. In certain embodiments, the cell is in a subject such as a research animal or human patient, the composition is administered to said subject, and the sample obtained is a biological sample from said subject.

In certain embodiments of a method of this disclosure, the composition can be subsequently administered to the same cell or to another cell according to said adjustment to the dosing based on the ratio between the amount exon-skipped mRNA and the amount of corresponding full-length mRNA determined in step (iv). That is, the determination can be used to continue to administer to and/or treat the same cell, subject, or patient, or the determination can be used to set or adjust the dosing to be used on another cell, subject, and/or patient.

In certain embodiments, the subsequent dose and/or timing/frequency thereof to be administered and/or that is subsequently administered is adjusted to achieve a level of reduction of the full-length mRNA of not more than about 90%, 80%, 70%, 60%, 50%, 40%, 30%, 20%, or 10%. In certain embodiments, the subsequent dose and/or timing/frequency thereof to be administered and/or that is subsequently administered is adjusted to achieve a level of reduction of the full-length mRNA of between any of about 90%, 80%, 70%, 60%, 50%, 40%, 30%, or 20% to any of about 80%, 70%, 60%, 50%, 40%, 30%, 20%, or 10%. In certain embodiments, the subsequent dose and/or timing/frequency to be administered and/or that is subsequently administered is adjusted to achieve a ratio of the amount of exon-skipped mRNA to the amount of full-length mRNA of about 1:99, 2:98, 3:97, 4:96, 5:95, 10:90, 20:80, 25:75, 30:70, 40:60, 50:50, 60:40, 70:30, 75:25, 80:20, 90:10, 95:5, 96:4, 97:3, 98:2, or 99:1. In certain embodiments, the subsequent dose and/or timing/frequency to be administered and/or that is subsequently administered is adjusted to achieve a ratio of the amount of exon-skipped mRNA to the amount of full-length mRNA of about 1:99, 2:98, 3:97, 4:96, 5:95, 10:90, 20:80, 25:75, 30:70, 40:60, 50:50, 60:40, 70:30, 75:25, 80:20, 90:10, 95:5, 96:4, 97:3, or 98:2 to any of about 2:98, 3:97, 4:96, 5:95, 10:90, 20:80, 25:75, 30:70, 40:60, 50:50, 60:40; 70:30, 75:25, 80:20, 90:10, 95:5, 96:4, 97:3, 98:2, or 99:1.

Provided for herein is a method of treating a disease or a medical condition (e.g, inhibiting pain receptors) with an exon-skipping inducing compound. In certain embodiments, the method comprises:
(i) administering to a subject in need of treatment a dose of a composition of this disclosure to induce exon-skipping of a target pre-mRNA and production of an exon-skipped mRNA therefrom. In certain embodiments, the compound reduces, but does not abolish, the amount of full-length mRNA expressed from the target pre-mRNA;
(ii) obtaining a biological sample from the subject; and
(iii) measuring in the sample the amount of the exon-skipped mRNA.

Certain embodiments of treating a disease or a medical condition further comprise (iv) measuring in the sample the amount of the corresponding full-length mRNA and determining the ratio between the amount of exon-skipped mRNA and the amount of corresponding full-length mRNA.

In certain embodiments, the dosing of the composition is adjusted to be subsequently administered based on the ratio between the amount exon-skipped mRNA and the amount of corresponding full-length mRNA. In certain embodiments, the composition is subsequently administered to the same subject or to another subject according to said adjustment to the dosing based on the ratio between the amount exon-skipped mRNA and the amount of corresponding full-length mRNA. In certain embodiments, the subsequent dose and/or timing/frequency to be administered and/or that is subsequently administered is adjusted to achieve a level of reduction of the full-length mRNA of not more than about 90%, 80%, 70%, 60%, 50%, 40%, 30%, 20%, or 10%. In certain embodiments, the subsequent dose and/or timing/frequency to be administered and/or that is subsequently administered is adjusted to achieve a level of reduction of the full-length mRNA of between any of about 90%, 80%, 70%, 60%, 50%, 40%, 30%, or 20% to any of about 80%, 70%, 60%, 50%, 40%, 30%, 20%, or 10%. In certain embodiments, the subsequent dose and/or timing/frequency to be administered and/or that is subsequently administered is adjusted to achieve a ratio of the amount of exon-skipped mRNA to the amount of full-length mRNA of about 1:99, 2:98, 3:97, 4:96, 5:95, 10:90, 20:80, 25:75, 30:70, 40:60, 50:50, 60:40, 70:30, 75:25, 80:20, 90:10, 95:5, 96:4, 97:3, 98:2, or 99:1. In certain embodiments, the subsequent dose and/or timing/frequency to be administered and/or that is subsequently administered is adjusted to achieve a ratio of the amount of exon-skipped mRNA to the amount of full-length mRNA of about 1:99, 2:98, 3:97, 4:96, 5:95, 10:90, 20:80, 25:75, 30:70, 40:60, 50:50, 60:40, 70:30, 75:25, 80:20, 90:10, 95:5, 96:4, 97:3, or 98:2 to any of about 2:98, 3:97, 4:96, 5:95, 10:90, 20:80, 25:75, 30:70, 40:60, 50:50, 60:40; 70:30, 75:25, 80:20, 90:10, 95:5, 96:4, 97:3, 98:2, or 99:1.

In certain embodiments, the initial dosing of an exon-skipping compound produces a high initial change in exon-skipped mRNA. The amount of exon-skipped mRNA, however, may wane over time. For example, for the first week there may be a 90% change, then 88%, then 80, etc over the course of a weeks or months. When the amount of exon-skipped mRNA (or, e.g., the corresponding ratio to full-length mRNA) falls back down to a certain level, it may be an indication that the subject needs to be dosed again with the exon-skipping compound.

In certain of any embodiments of this disclosure that involve the administration of a exon-skipping compound, the administration of the composition reduces the amount of full-length protein produced from the target pre-mRNA/from a gene of the target pre-mRNA. It will be understood by one of ordinary skill in the art that full-length protein is in reference to a functional protein. Such method can further comprise measuring the amount of the full-length protein and/or the reduction in the amount of full-length protein. For reasons such as explained in more detail elsewhere herein, in certain embodiments the subsequent dose to be administered and/or that is subsequently administered is adjusted to achieve a level of reduction of the full-length protein of not more than about 90%, 80%, 70%, 60%, 50%, 40%, 30%, 20%, or 10%. In certain embodiments, the subsequent dose to be administered and/or that is subsequently administered is adjusted to achieve a level of reduction of the full-length protein of between any of about 90%, 80%, 70%, 60%, 50%, 40%, 30%, or 20% to any of about 80%, 70%, 60%, 50%, 40%, 30%, 20%, or 10%.

In certain embodiments, the dosing of the composition administered is increased and/or is more frequent, thus increasing the amount of exon-skipped mRNA produced, decreasing the amount of full-length mRNA produced, increasing the ratio of exon-skipped mRNA/full-length mRNA, and/or decreasing the amount of full-length protein produced. By dosing of the composition administered is increased, it is meant that the amount of composition administered at a time point is increased. By dosing of the composition administered is more frequent, it is meant that the time between administrations is reduced. In certain embodiments, the dosing of the composition administered is increased and/or is more frequent, thus decreasing the amount of the protein produced from the gene of the target pre-mRNA.

In certain embodiments, the dosing of the composition administered is decreased and/or is less frequent, thus decreasing the amount of exon-skipped mRNA produced, increasing the amount of full-length mRNA produced, decreasing the ratio of exon-skipped mRNA/full-length mRNA and/or increasing the amount of full-length protein produced. By dosing of the composition administered is decreased, it is meant that the amount of composition administered at a time point is decreased. By dosing of the composition administered is less frequent, it is meant that the time between administrations is increased. In certain embodiments, the dosing of the composition administered is decreased, thus increasing the amount of the protein produced from the gene of the target pre-mRNA.

Certain embodiments provide for a plurality or a series of compounds that used in combination to target two or more different genetic pathways associated with a disease state or condition. The compounds can be administered to the same patient either simultaneously or near simultaneously or at different times. In certain embodiments, the effect of at least one of the compound on the production of exon-skipped mRNA, full-length mRNA, functional protein, and relationships between them are measured and determined. For example, certain diseases involve more than one protein and/or molecular pathway. Certain embodiments of this invention target selective intervention of one or more of them using the compositions and methods described herein.

As disclosed above, certain methods of this disclosure include measuring the amount of exon-skipped mRNA and/or measuring the amount of full-length mRNA. In certain embodiments, the amounts can be measured using standard molecular biology techniques such as polymerase chain reaction (PCR), nucleic acid sequencing, oligonucleotide ELISA, and/or mass spectrometry.

As disclosed above, certain methods of this disclosure include administering a composition to a cell, subject, research animal, or human patient. In certain embodiments, the administered composition comprises a pharmaceutically acceptable carrier or diluent. In certain embodiments, the composition is administered orally, locally, systemically, e.g., subcutaneously, perineurally, etc. In certain embodiments, the method comprises targeting a

### EXAMPLES

### Example 1

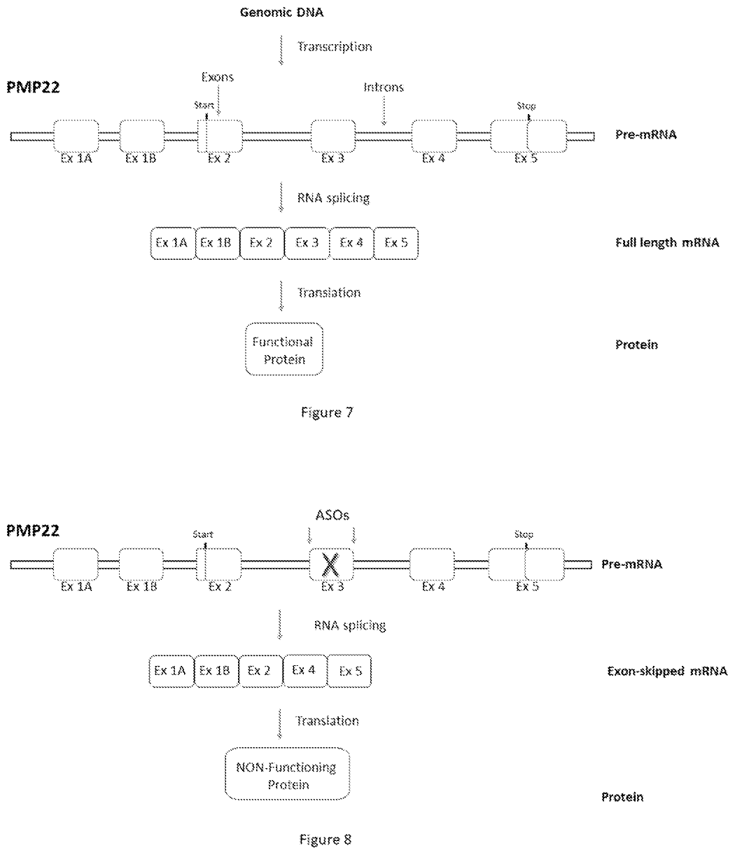

Consider the PMP22 gene that is present in animal cells. The PMP22 gene codes for the PMP22 protein. Certain disease states (such as CMT1A for example) result for the duplication of the PMP22 gene (two copies for CMT1A) and thus overproduction of the PMP22 protein by a factor of ×2. Production of PMP22 protein has also been shown to be associated with certain cancerous disease states. Thus, it is desirable to reduce the production of PMP22 protein in these patients. In CMT1A patients, it may be desirable to return these patients to a normal protein level as healthy non-CMT1A patients, thus lowering the amount of PMP22 production by 50%. Since PMP22 protein is a required protein in regular physiological function, it is desirable to down regulate a portion of the production but not lower the production to a minimal level that may cause other side effects (such as a distinct neuropathy called hereditary neuropathy with predisposition to pressure palsy (HNPP), or loss of peripheral nerve function). The PMP22 Gene is shown in FIG. 7. his gene has 6 different exon regions (TA-5) with corresponding intros separating the exons at the pre-mRNA level. Exons 2-5 code for the amino acids that makeup the full-length PMP22 protein between the start and stop regions shown. During normal activity, the introns are removed during transcription (or splicing) and produce full-length mRNA. Ribosomes then convert the mRNA to functional protein.

mRNA silencing approaches previously described partially of fully eliminate the entire production of mRNA and those corresponding protein. To monitor their effectiveness, the total mRNA levels before treatment and after must be compared to determine relative effectiveness of the compounds and amount used. Typically, these techniques targe the pre-mRNA at a regulatory level (eliminate it completely per binding event) or mRNA after transcription (through binding compounds or degradation strategies).

In this illustrative example, the pre-mRNA is targeted with selected compounds that allow the production of the mRNA but promote an exon to be “skipped” during the translation process, producing a exon-skipped mRNA that can be monitored. Thus, both full-length mRNA (representing pre-mRNA where the compound did not bind or was ineffective) and “exon-skipped” mRNA can be monitored and compared to determine the drug's effectiveness. The outline is shown if FIG. 8.

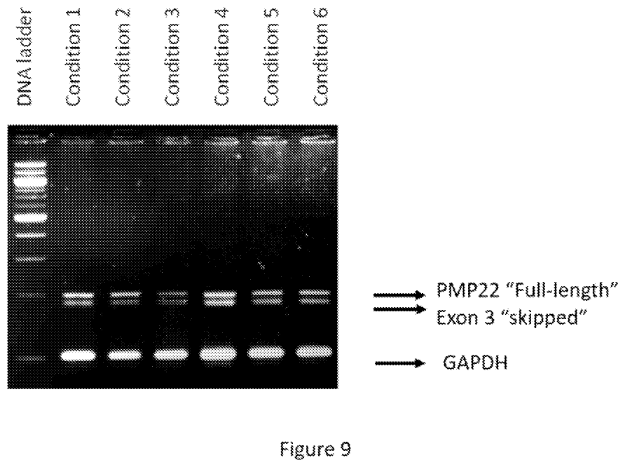

For this example, a variety of synthetic anti-sense oligonucleotides were designed that were complimentary to the intron-exon regions (both 3′ and 5′ regions around the exons) of Exons 3 and 4 of the PMP22 gene and were designed to elicit an exon skipping event of their corresponding target exon. These were synthesized using morpholino backbone chemistry to enhance binding to the target pre-mRNA and provided biological stability. One PMO (SCH-012 20-mer (SEQ ID NO: 75)) was designed to bind to the 3′ end of the exon 3/intron junction and elicit “skipping” of Exon 3 during splicing. For SHC-012, the target area of the PMP22 genome in this region is 5′-CGgtgaggctggttttgtgc-3′ (SEQ ID NO: 153) and the corresponding complimentary PMO sequence is 5′-gcacaaaaccagcctcacCG-3′ (SEQ ID NO: 75). In this nomenclature, bases that are part of Exon 3 are noted in the target region as capital letters and bases in the intro region as lower case letters. These compounds were purchased from Gene-tools LLC (Philomath OR). They were transfected into HEK-293 cells in a cellular assay (these cells contain humanized PMP22 genes). PCR primers were designed to span the entire region from Exon 2 to Exon 5 of the mRNA, thus the size of the resulting PCR products would change if the target mRNA contained Exon 3 or Exon 3 was skipped, but mRNA was still synthesized. FIG. 9 shows the result of one such experiment.

In this example, 6 different experimental condition were used to “mimic” biological variation from animal to animal, time of day, etc. while the amount of ASO SHC-012 was kept at a maximum amount to reach equilibrium. More specifically, all of the conversion from PMP22 mRNA should be the same for all conditions even though the cells are differentially expressing total protein. Software was used to quantitate each of the band from the gel in FIG. 9 and shown in Table 1.

TABLE 1

| Condition | 1 | 2 | 3 | 4 | 5 | 6 | Control |
|---|---|---|---|---|---|---|---|
| Band Integration | 141.7 | 91.2 | 38.4 | 152.0 | 96.9 | 90.9 | 189.0 |
| Band Integration | 85.4 | 36.6 | 30.1 | 126.0 | 69.7 | 65.0 | |
| Change (full lenth only) | 25% | 52% | 80% | 20% | 49% | 52% | |
| Change (ratio) | 38% | 29% | 44% | 45% | 42% | 42% | |

As can be noted in Table 1, if only PMP22 full-length mRNA production is monitored (as is capable with previously described technologies), the measured effect of the protein production capacity of the cells varies from 25% to 80%. If the ratio of the two bands is measured, this variance shrinks to 29-45%. The lower band (not present in control samples) was sequenced and confirmed to be full-length PMP22 with Exon 3 missing. This example shows the benefit of producing a non-naturally occurring exon-skipped mRNA and comparing the amount of this skipped mRNA to the full-length mRNA to overcome experimental variance and achieve more accurate results of drug effectiveness.

### Example 2

Additional morpholino anti-sense oligonucleotides were designed to bind to various portions of the intron to Exon 3 and intron to Exon 4 junctions of the PMP22 pre-mRNA to elicit exon skipping. The compounds designed are shown in FIG. 10A,B (for Exon 3 skipping compounds) and FIG. 11A,B (for Exon 4 skipping compounds). The designed compounds are anti-sense oligonucleotides (ASOs) that bind to the intron/exon junctions (or near that junction) of each exon. The designed oligonucleotides were 25 base pairs long and will hybridize exactly to the corresponding region of the pre-mRNA. As one or ordinary skill in the art will understand, shorter ASO subsets can also be utilized as can ASOs with 1, or 2 or 3 base pair mismatches within the target region.

FIG. 10A shows compounds that overlap the region near the 5′ end of the Exon 3 intron border.

FIG. 10B shows compounds that overlap the region near the 3′ end of the Exon 3 intron border.

FIG. 11A shows compounds that overlap the region near the 5′ end of the Exon 4 intron border.

FIG. 11B shows compounds that overlap the region near the 3′ end of the Exon 4 intron border.

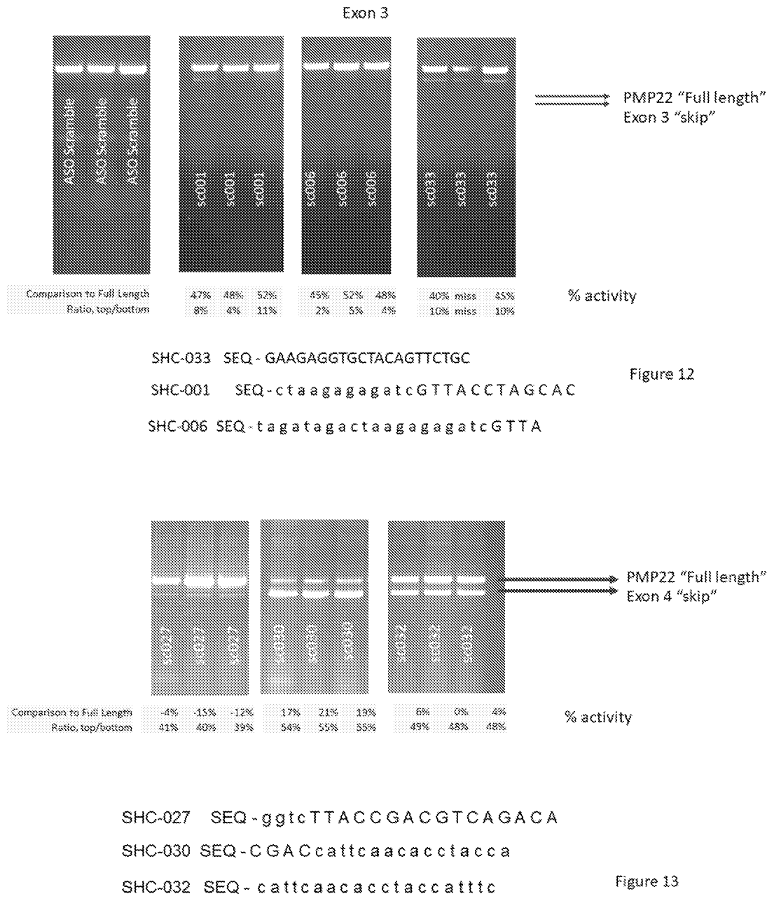

Select ASO compounds of various lengths are shown in FIG. 10 and FIG. 11 that were synthesized (using morpholino backbone chemistry, purchased from GeneTools LLC) and tested in cellular assays to determine their effectiveness and producing exon-skipped mRNA. These compounds were transfected into HEK-293 cells in a cellular assay (these cells contain humanized PMP22 genes). PCR primers were designed to span the entire PMP22 region, thus the size of the resulting PCR products would change if the target mRNA was full-length or contained skipping of target Exons was achieved (but mRNA was still synthesized in the presence of the compounds). Cellular assays were run for each compound and the corresponding gels (PCR results are shown in FIG. 12 and FIG. 13).

FIG. 12 shows the results from 3 compounds which were designed to skip Exon 3 during mRNA production. As can been seen from the gels run after PCR amplification, bands corresponding to both full-length PMP22 mRNA and Exon 3-skipped mRNA are present (the Exon 3-skipped band was confirmed through sequencing to be exactly the full-length PMP22 mRNA minus the bases corresponding to Exon 3). To demonstrate the power of this result, and comparison to previous approaches (where no stable skipped exon mRNA is produced), the amount of full-length PMP22 for each condition was compared to the amount of full-length PMP22 in the control samples (see the table below the gels in FIG. 12). Other mRNA silencing approaches will only report these two ratios. The ratio of the amount of full-length PMP22 RNA/exon-skipped mRNA was also calculated on the same table. As can be noted, many of the results from samples show the incorrect value of drug activity (in some cases showing that the drug was ineffective or even increased the amount of PMP22 when full-length alone was reported) when only full-length mRNA is monitored. When the ratio of the two PCR products is considered, all effective drugs showed correct results and all drugs that were ineffective showed no activity. This can visually be seen from the gels themselves and is noted in the table where the band of the gels were integrated. Specifically, all 3 compounds showed very similar activity (˜50%) using the traditional method of analysis even though the gels clearly show compound SHC-033 to be the most active compound within this group (even though it shows the lowest activity using the traditional means). Lane 2 for SHC-033 was not analyzed due to a poor injection on the gel, indicated by the poor band structure within that lane.

FIG. 13 shows the same approach utilized for compounds designed to skip Exon 4 during conversion from pre-mRNA to mRNA. In this example, different gel analysis conditions were utilized (compared to FIG. 12) so the integration of each gel peak was normalized against background. The resulting analysis is shown in FIG. 13 below each gel. The amount of full-length pre-mRNA was compared to control samples (not shown) to mimic previously described methods where no exon-skipped mRNA is present and measurable. For these compounds and experimental conditions, the traditional comparison showed negative activity for compound SHC-027 (meaning the full-length PMP22 went up after the addition of the drug) and the other results are shown. The activity for compound SHC-032 showed near zero activity using this method. In contrast, this example—where the comparison of the two RNA bands is made and the control sample is not used in the calculation—demonstrate a clear trend that correlates well to actual activity. The lower band in these gels was confirmed by sequencing and is the full-length pmp22 mRNA minus the bases for Exon 4.

### Example 3—Monitoring Exon 3 Skipping in Animal Models

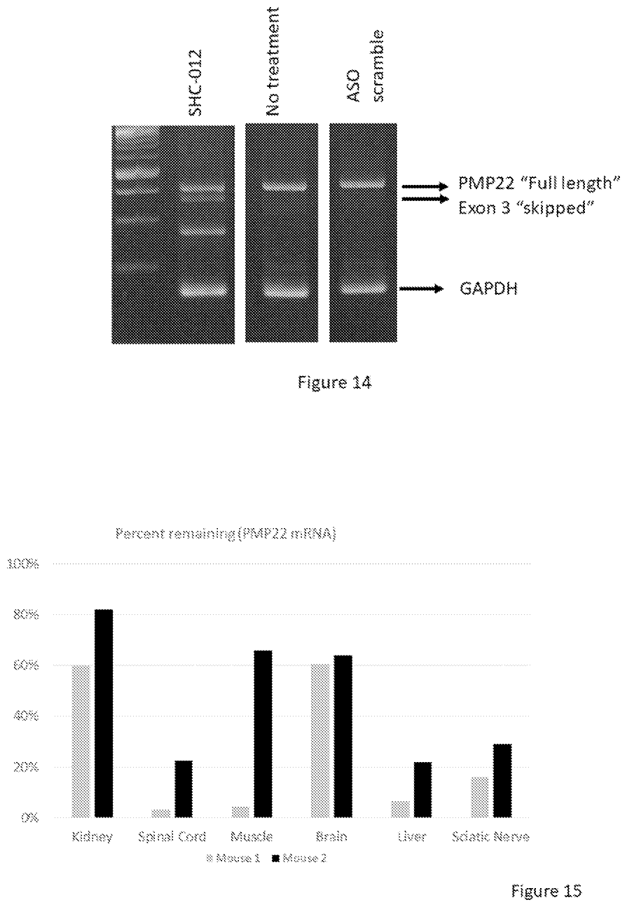

An important aspect of this disclosure is that the exon-skipping drugs disclosed can perform exon skipping in live animals. In vivo activity was tested using C3 mice (3 copies of human PMP22). C3 mice were bred and genotyped according to the published protocol from Jackson Labs. At 5 weeks, animals were injected subcutaneously with a single 6.7 mg of SHC-012 or a scramble control PMO. Untreated C3 mice were included as a control. Animals were sacrificed after 24 hours after the injection and tissues were collected (sciatic nerve, kidney, liver, brain, spinal cord, etc.) and analyzed for PMP22 mRNA reduction/expression and the exon-skipped version of the PMP22 mRNA. RT-PCR was used to evaluate PMP22 expression (3% agarose gel). GAPDH was included in the reaction as a control to allow for comparison between lanes (FIG. 14). As the liver will naturally receive a large dose of any peripherally delivered therapeutic, liver PMP22 expression was examined and accumulation was found of exon-skipped PMP22 products with a reduction in full-length PMP22 expression (FIG. 14). The SHC-012 animal has a significant reduction in PMP22 full-length mRNA and a measurable amount of Exon 3-skipped PMP22 mRNA (confirmed by sequencing).

Additionally, other tissues from these animals were analyzed by the same PCR methods and gel analysis (FIG. 15) and compared the full-length and skipped exon amounts to determine overall reduction in these tissues. The evidence demonstrates PMP22 reduction and/or exon-skipping in several tissues, including peripheral nervous tissues (sciatic nerve) from C3 mice with a single injection (in the small of the back).

Importantly, when peripheral nervous tissue was examined (e.g., surrogate for in vivo Schwann cells), a clear reduction in full-length PMP22 expression was observed. It is noted that the C3 mice express the human genomic PMP22 gene which allows ASOs to target important regulatory sequences in and around the various exons.

### Example 4

Another aspect of the current invention is that the drug compounds proposed not only produce an exon skipped mRNA in animals (that is measurable), but they also elicit a phenotypic response in the animals.

C3 mice (3× human PMP22 gene) were selected as the animal model since they have been shown to exhibit CMT like behaviors, have been well studied, and most importantly for this project, express the human genomic PMP22 gene (Huxley C, Passage E, Manson A, Putzu G, Figarella-Branger D, Pellissier J F, Fontes M. Construction of a mouse model of Charcot-Marie-Tooth disease type IA by pronuclear injection of human YAC DNA. Human molecular genetics. 1996; 5(5):563-9. Epub 1996 May 1. doi: 10.1093/hmg/5.5.563. PubMed PMID: 8733121). This is essential since the ASOs rely on exact sequence identity within the exon and intronic regions.

Two animal groups were studied: 5-week old animals (early symptomatic disease stage exhibiting partial behavioral changes already) and 3-day old animals (that received treatment prior to measurable CMT behavioral changes). SHC-012 was injected for all animals. At 5 weeks, C3 animals (n=3) were injected subcutaneously with 3.3, 17, or 50 mg/kg of SHC-012 or a scramble control PMO once/week for 5 weeks. A second set of C3 mice were also tested where the initial injections were performed (SQ on the upper back) starting at 3 days of age (to simulate prior to disease onset in humans) with weekly injections occurring equal to 1 mg/kg and 10 mg/kg per animal (for once/week for 12 weeks). Untreated (n=4) WT mice were included as a control and tested as the others. No adverse reactions were observed, and all animals remained healthy through the study period, gaining weight at similar rates throughout all dose ranges and showed no overt signs of toxicity. As anticipated, all animals recovered smoothly, rapidly and without complication from each injection and showed no immediate or delayed ambulatory, behavioral or neurological defects from the injection procedure.

To allow for a training and acclimation period, all animals started testing procedures at 4 weeks of age (˜×4 per week) using a standard rotarod mechanical system and a dowel walk one week prior to initial injection. For the dowel walk, a cylindrical 10 mm wooden dowel was suspended and the mice allowed to walk (unaided and undirected) from one end of the setup to the other. The time to traverse, number of slips during the walk, and any falls during the walk were recorded. The initial training period was not used for data analysis. After injection, animals continued to undergo daily testing to monitor overall balance, leg performance, tail stability, etc. which have been shown to be measurable phenotypic responses to disease progression in this model (Huxley C, Passage E, Manson A, Putzu G, Figarella-Branger D, Pellissier J F, Fontes M. Construction of a mouse model of Charcot-Marie-Tooth disease type 1A by pronuclear injection of human YAC DNA. Human molecular genetics. 1996; 5(5):563-9. Epub 1996 May 1. doi: 10.1093/hmg/5.5.563. PubMed PMID: 8733121). As previously reported, untreated animals began to exhibit very noticeable walking problems between 6-8 week of age.

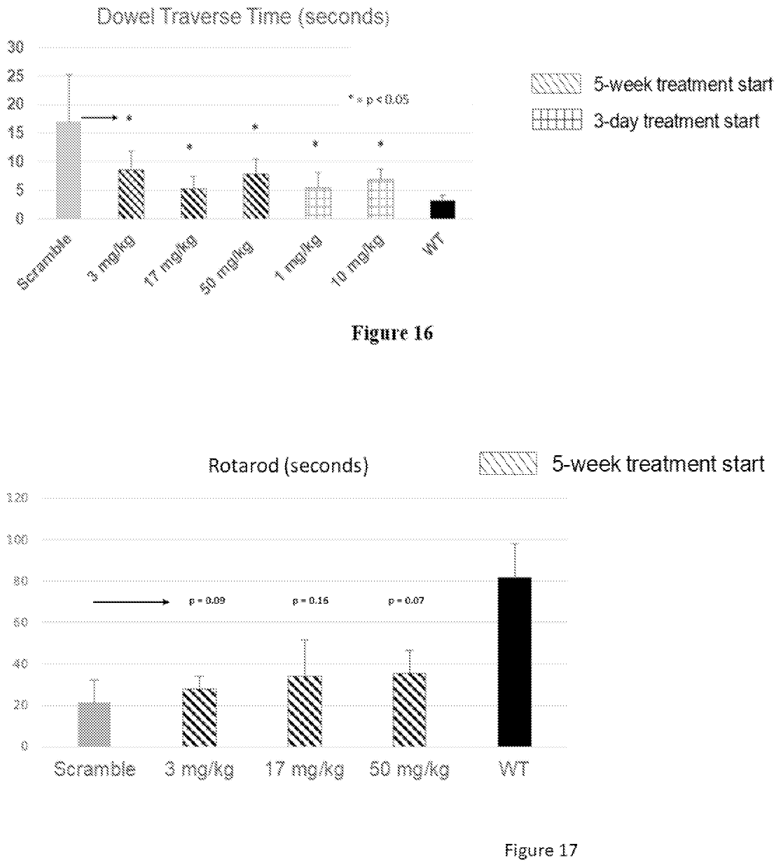

All of the data (from both test groups) was analyzed at 12-weeks of age for all animals for direct comparison, to determine the time required to traverse the dowel, as well as the number of slips, falls, hesitation, etc. Untreated animals learned to traverse more slowly over time and had less slips as the experiments progressed but visibly were walking more carefully often “hugging” the dowel. The videos demonstrate a very clear difference between treated and untreated animals with noticeable behavioral differences. FIG. 16 shows the performance (phenotypic) data for animals studied as compared to control and wildtype by comparing the amount of time required to walk across the dowel unaided. The number of slips (and falls) on the dowel showed a similar trend. Five (5) consecutive testing days were analyzed at ˜12-weeks of age to compute averages and standard deviations across all animals within the dosing group for time across the dowel and number of slips. Rotarod testing was also performed but showed a much higher variance per test. The selected concentrations were initially chosen (based on the cellular data and our previous experience with biodistribution of similar molecules) to achieve a low dose (3 mg/kg), medium dose (17 mg/kg), and high dose (50 mg/kg), with the understanding that completely eliminating PMP22 would be deleterious. All animals performed significantly better than the control group (also highly noticeable from the video data) with the 17 mg/kg (5 week) and both groups of animals at P3 performing near wild type animals.

Animals throughout the study were also tested on a commercial rotarod system and the performance data is shown in FIG. 17.

For both experiments, the p values were calculated (versus the scramble control animal) and are shown in FIG. 16 and FIG. 17.

The data from Examples 2 and 3 clearly indicate the alteration of the PMP22 mRNA by SCH-012 as described in the invention has the desired phenotypic effect of the animals used. Namely to make them walk better and alleviate certain symptoms caused by their overproduction of PMP22 protein.

Surprisingly, this approach shows a much greater effectiveness at alleviating symptoms in CMT1A mouse models than the traditional knockdown approach. As an example, referring to FIG. 1D of the paper by Zhao et al. (The Journal of Clinical Investigation, Volume 128, No 1., January 2018, pp 359-368), weekly injections of 100 mg/kg of their most potent ASO which targets the 3′ UTR of the human PMP22 gene into C22 humanized CMT1A mouse models (containing 7 copies of the human PMP22 gene) were required to obtain statistically significant results in rotarod testing. No statistical difference was determined was obtained with 50 mg/kg per week. For the present analysis of animals studied starting at the same age, significant improvement was noted with animals injected as low as 3.3 mg/kg per week. For animals that started treatment at a younger age, only 1 mg/kg was required (see FIG. 16 and FIG. 17). While the C3 animal model used here has only 3 copies of the PMP22 gene and the C22 animals had 7 copies (a 2.3 fold increase of target RNA), 50 mg/kg is 15 fold higher than 3.3 mg/kg and 50 fold higher than the 1 mg/kg animals. Clearly, the exon skipping approach described here has a significant advantage over the 3′ UTR strategy in terms of efficacy per dose.

Improving the critical disease-associated pathology in axons is an important surrogate marker of efficacy. To determine if the molecular change observed in PMP22 resulted in an improvement in axonal pathology, a variety of axons from wild-type were examined, treated, and scramble-treated C3 mice. Animals from the 17 mg/kg treatment group, wild-type animals, and scramble control group C3 animals were sacrificed at 20 weeks of age (6 weeks after the treatment stopped for the 17 mg/kg treatment group above. Nerves were removed from all of the animals (sciatic, peroneal, ulnar, and tibial nerves) and cross-sectional portion of the nerves were prepared for Transmission Electron Microscopy (TEM) analysis.

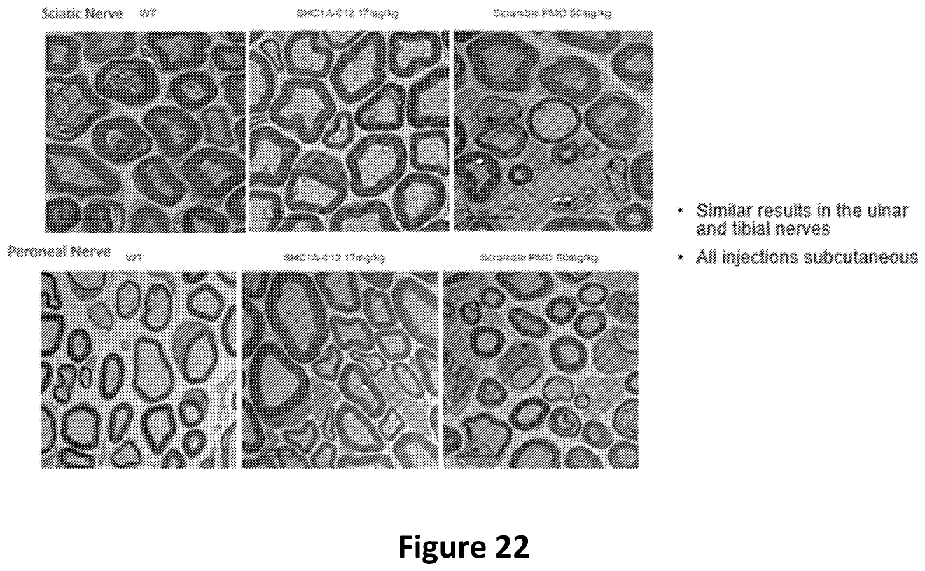

FIG. 22 (top) shows images of the sciatic nerve for WT, untreated and treated animals; FIG. 22 (bottom) shows images from the peroneal portion of the nerve.

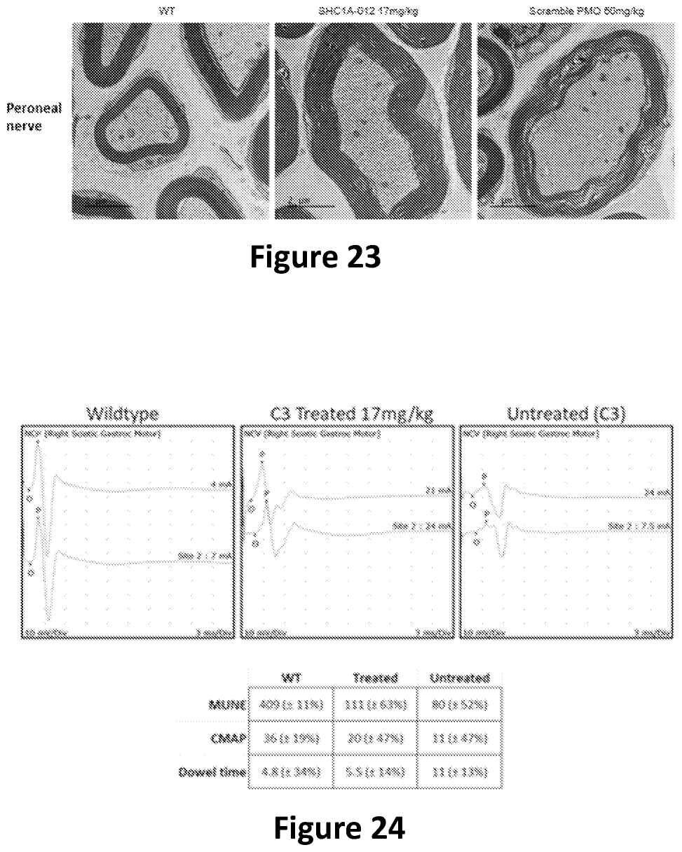

FIG. 23 is a TEM image at higher magnification of portions of a Peroneal nerve from each animal.

Analysis of the sections revealed that SHC-012 treatment significantly improved the pathology from multiple nerves (sciatic is shown) compared to scramble-treated axons based upon demyelination, thickness, structural integrity and the frequency of dying axons. Wild-type tissues were still better than the SHC-012 treated axons, however, it is clear that the ASO was improving these important structural hallmarks of disease. Similar results were also seen when sections from the ulnar and tibial nerves were analyzing, indicating that the subcutaneous injections used (on the scruff of the animals backs) were effectively distributing the molecules throughout the animal's bodies sufficiently to have a positive impact.

Based upon the improvement in the myelin sheath, the functional impact of this improvement was next examined. Electrophysiology was performed to measure functional recovery in C3-treated mice. In this cohort of treated C3 mice (and untreated C3 mice as a control) from the same treatment group described above, improvements in the CMAP and MUNE were detected in the treated mice (FIG. 24).

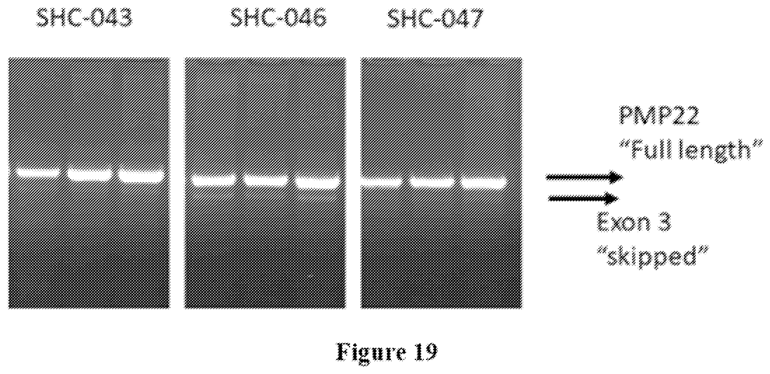

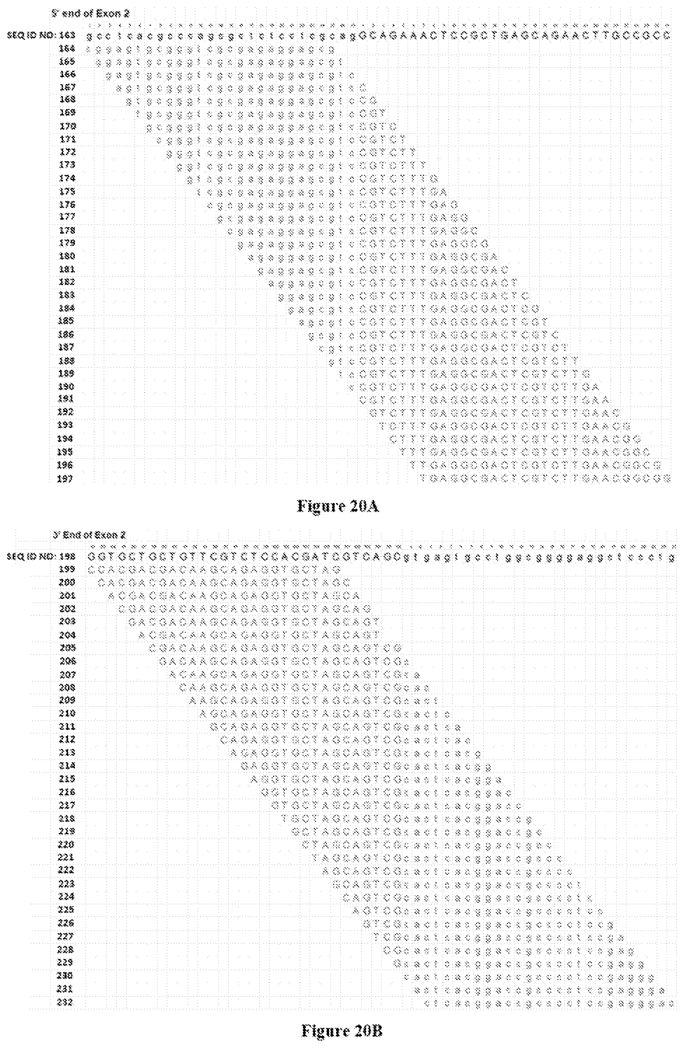

In FIG. 24 (top), electrophysiology plots from the sciatic gastric section of sedated mice are shown. The table at the bottom of FIG. 19 shows the average values of MUNE and CMAP from 3 measured animals per group. The “dowel time” which is a measure of general fitness, balance and mobility (described below) is included and illustrates that improved dowel performance tracks with improved electrophysiological measures.

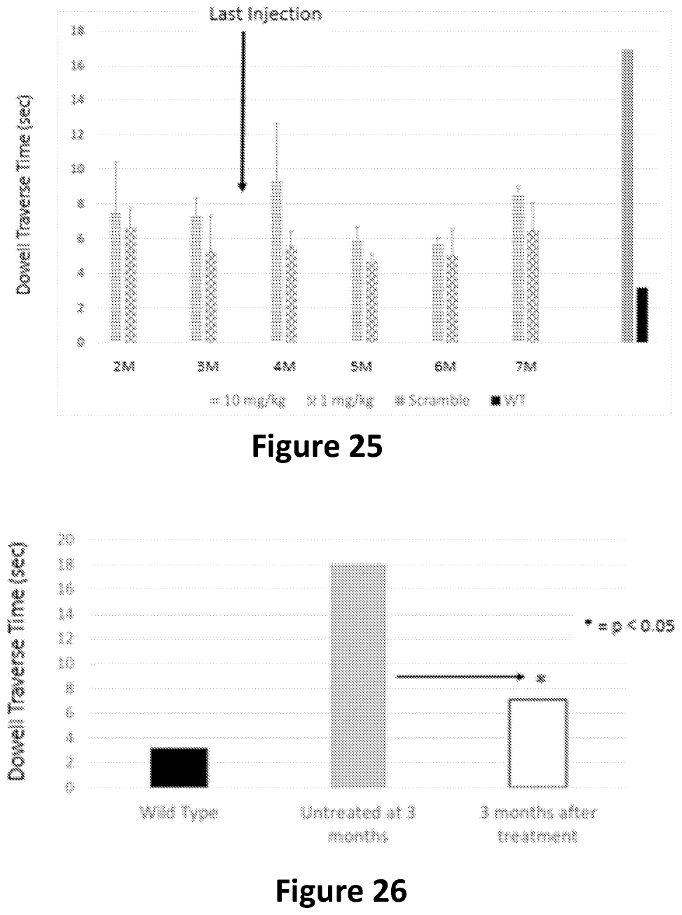

A key specification of the envisioned CMT1A treatment is that treatment will only be required 2-4 times per year for each patient. This is potentially possible because PMO ASOs are resistant to nucleases and are very stable once cellular uptake has occurred. To examine the duration and durability of SHC-012 activity, animals from the 1 mg/kg treatment and 10 mg/kg treatment groups above were monitored for 5 months after their last treatment (FIG. 25). The half-life of PMO molecules (once they reach cells such as Schwann cells) is ˜3-4 months for most tissue; thus, our hypothesis was that treatment benefit would persist for extended periods of time. Remarkably, the C3 animals at 5 months post-treatment have continued to perform well on the dowel test, with little or no reduction in activity after 4 months from last treatment (extended testing is still ongoing).

FIG. 25 shows the results for both treatment groups (with the scramble animals' group and wild-type animals shown for comparison at 4 months of age). Each data set of the histogram is an average of 4 days dowel travers time at the end of each month. Thus, the last data set plotted is for animals that are 7 months old.

### Example 5

Another contemplated embodiment of the present disclosure is to monitor the amount of exon-skipped mRNA in blood from an animal after drug injection. For example, SHC-012 can be injected to produce Exon 3-skipped mRNA (other compounds can be envisioned). In this example, animals do not need be sacrificed or biopsied to periodically monitor the effect of the drug on full-length mRNA, and thus protein production, and thus phenotypic response. Consider an experiment similar to Example 5. Blood samples can be taken from mice prior to injection of the drug and the amount of full-length PMP22 in the blood stream and the amount of exon-skipped PMP22 can also be monitored. In this example, the level of full-length mRNA will be measurable in the blood but vary. The level of exon-skipped mRNA will be proportional to the number of errors that occur during mRNA production. After the drug is given, blood can periodically be drawn from the animals and both levels of mRNA periodically checked in a quantitative manner. For example, quantitative PCR can be performed on the blood samples after sample preparation using methods as described by the Biodrop system from BioRad Inc. (Hercules CA) which is very sensitive to low levels of RNA present in samples and produces a quantitative result depending on the specific reagents used. Reagents can be used that produced a single if the RNA to be detected contains the exact sequence of Exon 2 connected to Exon 3 (only present in full-length PMP22 mRNA) and different reagents can be used that only produce signal in the presence of Exon 2 connected directly to Exon 4 (only present in Exon skipped mRNA). The amount of Exon 3-skipped mRNA or the ratio of full-length mRNA to Exon 3-skipped mRNA can be assessed in the blood and correlated the amount of mRNA skipped in the target tissues to monitor the effectiveness of the drug over time.

### Example 6

A number of phosphorodiamidate morpholino oligomer PMOs (using morpholino backbones) were designed and synthesized that did not contain a continuous sequence against the PMP22 pre-mRNA, but rather contained bridging portions as described above. FIG. 18 shows the sequence of the 5′- and 3′-ends of Exon 3 at the top, along with the intro portions that are adjacent to the Exons. A gap appears in the top (with the word Space and “/” below) indicating that the other portions of Exon 3 are not shown that would join position 20 (a G) with position 86 (an C). PMOs SHC-043 (SEQ ID NO: 156), SHC-044 (SEQ ID NO: 159), and SHC-045 (SEQ ID NO: 162) were designed as shown against portions of both the 3′- and 5′-ends. These PMOs are continuous molecules (as seen in the FIG. 18) but will hybridize to non-contiguous portions of the PMP22 pre-mRNA. Other bridging ASOs are also envisioned, provided they can hybridize to PMP22 pre-mRNA and induce both the production of an exon-skipped mRNA product and reduction of the full-length mRNA.

These compounds were used in cellular assays (as described above) to test their performance and the results are shown in FIG. 19, again showing both the full-length and exon-skipped product that can be separately quantitated to determine activity.

### Example 7

A number of ASOs were designed for exon skipping of Exon 2 in the PMP22 pre-mRNA (FIG. 20). As disclosed elsewhere herein, shorter length and mis-matched ASOs are also contemplated. These ASOs were designed at the 3′- and 5′-end of Exon 2.

### Example 8

For CMT1A, it is desirable to monitor the activity at a molecular level of a given drug over time without having to sacrifice the animal (during research studies) or perform a major biopsy on a human patient, in order to access the tissue of interest and determine if the pre-mRNA, mRNA, or protein for PMP22 has been affected. CMT1A is a disease where the peripheral nerve tissue is the desired tissue to affect the level of PMP22 and within this tissue the myelinated sheets around the tissue and the PMP22 protein is largely produced within the Schwann cells within this region. Thus, in order to effective measure the effect of any drug on the Schwann cells, a major tissue biopsy must be performed and/or the animal must be sacrificed in order to assess these tissues. These measurements are not practical or possible in human patients during drug clinical trials or treatment. Thus, measuring the activity over time is not possible in humans and difficult in animals. Often, for laboratory experiments, large groups of animals will be studied and periodically sacrificed to assess these tissues.

While the majority of the pre-mRNA and mRNA for PMP22 is located within the cells and tissue of interest, a very small, but detectable amount of both materials will be present in the blood stream or other biological fluids that are accessible without major biopsy or autopsy. This material in the blood (while not synthesized there), will be present from cells within the body dying and RNA leaking out into the blood stream non-specifically. Other materials may be present from damaged cells or non-specific cellular expulsion. For previously described drug approaches where the total mRNA is reduced as the mode of action of the drug, it is impractical to measure a slight lowering of total mRNA in the blood stream from the effect of the drug in a large background variance due to natural fluctuations (from exercise, food, metabolism, disease, damage, etc.). These variance in total natural mRNA can be quite large over even a 24 hour period, thus quantitating a small decline due to the drug is impractical. In certain embodiments, there will be nearly zero amount of exon-skipped PMP22 in the blood stream without the presence of an exon-skipping drug. Thus, if the amount of exon-skipped mRNA is quantitated in the blood stream, it will be directly proportional to the activity of the drug. Even more quantitative will be to compare the ratio of the amount of full-length PMP22 mRNA to exon-skipped mRNA within the blood stream at any given time to confirm and quantitate the drug activity. For example, a day after a drug is given to a patient, the drug has had sufficient time to be effectively distributed throughout the body and into the tissues and cells of interest. A near maximal amount of new PMP22 protein production at the cellular level should be achieved for a given drug composition and dose at this time. The concentration of exon-skipped mRNA in the blood than can be periodically monitored from the blood over time and correlated to drug activity. Even more preferrable would be to quantitate both the full-length and exon-skipped mRNA and correlate that ratio to drug activity. This method gives an internal control to the measurement which simplifies that assay measurement and increases accuracy. As that value goes down over time (indicative of new cells being created that do not contain the drug, loss of activity of the drug, etc.), an additional dose of the drug chosen can be given at that time to re-equilibrated exon skipping to the desired value.

The method of this disclosure of measuring the exon-skipped mRNA in the blood can also be used to adjust the initial dose on a patient-by-patient basis or during dose escalation studies during clinical trials. For CMT1A (and other diseases) the variance in actual measurements (versus true molecular biology activity) could be disastrous for certain patients. Incorrect results could lead to underdosing of patients (thus missing the drug activity window) or significant overdosing of patients (leading to potentially dangerous side effects). Due to a number of factors, two different patients may require different amounts of drug to achieve desired levels of PMP22 reduction. For instance, if it is desirable to block 50% of PMP22 protein production, a physically smaller patient may require less drug than a heavier patient. Patients with different metabolisms or lifestyles may also require different amounts of drug per treatment to achiever optimal molecular changes. By producing a stable mRNA product within the animal or patient that is not naturally occurring (as described in this disclosure), these measurement are made possible.

### Example 9

A key consideration for the treatment of CMT is to determine how the treatment may be effective for older patients that have progressed in the disease for a longer period of time and may have more axon damage than other patients. C3 animals were allowed to progress until 3 months of age and monitored for their walking ability across a suspended dowel as described above. The amount of time to cross the dowel was monitored prior to their treatment. The animals where then treated with 17 mg/kg of SCH-012 weekly for 6 weeks then allowed to progress (untreated) for an additional 6 weeks and again monitored for walking capability.

FIG. 21 shows the results of this experiment (with 3 month old wild-type animals also plotted for comparison). As can be seen, the group of animals were performing very poorly at 3 months of age (average dowel travers time of 18.1 seconds with a standard deviation of 7.1 seconds). After treatment and a recovery period, the same animals significantly improved to and average walking time of 7.1 seconds with a standard deviation of 2.4 seconds). For each bar in the graph, animals were measured for 4 days (2 days apart) and the averages of those time points are shown.

### Example 10

Another group of C3 animals were allowed to progress untreated until 12 months of age. At 12 months of age, wild-type animals will also begin to show some degradation in walking and balance ability, so wild-type animals were also studied. At 12 months of age, C3 animals were treated (Table 2). For treatment groups 1, 2, 3, 5, 6, and 7, five animals were studied in each group and for groups 4 and 5, four animals were studied.

TABLE 2

| | Animal [symbol] | | Treatment | Testing | Testing | Testing | |
|---|---|---|---|---|---|---|---|
| GROUP | Type | Treatment | Days | Time 1 | Time 2 | Time 3 | Sacrifice |
| 1 | C3 | SHC1A-012 [symbol] mg/kg | Day [symbol] | Day 0 | Day 8 | | Day 8 |
| 2 | C3 | SHC1A-012 [symbol] mg/kg | Day [symbol] | Day 0 | Day 30 | | Day 30 |
| 3 | C3 | SHC1A-012 [symbol] mg/kg -2 | Day 1 | Day 0 | | Day 60 | Day 60 |
| 4 | C3 | SHC1A-012 [symbol] mg/kg - 2 | Day 1, Day 31 | Day 0 | Day 30 | Day 60 | Day 60 |
| 5 | C3 | SHC1A-012 5 mg/kg | Day 1, Day 31 | Day 0 | Day 30 | Day 60 | Day 60 |
| 6 | C3 | 50 mg/kg Scramble | Day 1 | Day 0 | | | |
| 7 | W [symbol] | None | None | Day 0 | | | |

[symbol]

indicates data missing or illegible when filed

The raw performance results of the testing are shown in Table 3. For the dowl walking time, the same apparatus was used as before, and the times (in seconds) were recorded per animal two days in a row and the average for the group calculated using both days. For grip strength, each data set is the front paw grip strength (in grams for a 25 Newton pull setting) for the entire group tested on one day.

TABLE 3

| | | Dowel Walking Time | | | Grip Strength | | |
|---|---|---|---|---|---|---|---|
| GROUP | Treatment | Time 1 | Time 2 | Time 3 | Time 1 | Time 2 | Time 3 |
| 1 | SHC1A-012 [symbol] mg/kg | 14.2 | 15.8 | | 53 | 54 | |
| 2 | SHC1A-012 [symbol] mg/kg | 15.1 | 11.2 | | [symbol] | 58 | |
| 3 | SHC1A-012 [symbol] mg/kg | 1 [symbol] .4 | 11.6 | | 40 | 56 | |
| 4 | SHC1A-012 [symbol] mg/kg - 2 | 16.1 | 10.8 | 7.3 | 20 | 38 | 72 |
| 5 | SHC1A-012 5 mg/kg - 2 | 14.3 | 13.9 | [symbol] .5 | 40 | 44 | 44 |
| 6 | 50 mg/kg Scramble | 16.5 | 8.8 | | 43 | 43 | |
| 7 | None | 7.2 | 6.5 | | 99 | 92 | |

[symbol]

indicates data missing or illegible when filed

Table 4 shows the data from Table 3 where the average percent improvement (compared to the first day for each group separately) is shown. For example, for Group 2 dowel walking time, the animals in the group showed on average a 26% improvement in walking performance from before treatment and after treatment (since a reduction in time is an improvement for walking). For the animals in groups 4 and 5 that had two performance testing days (30 days and 60 days), both time point percent improvements are compared to the original pre-testing data. For example, for Group 4 grip strength, the animals showed a 90% improvement after 1 treatment (Time 1-2) and a 256% improvement at the 60 day mark compared to the pre-treatment value. Positive increases in grip strength demonstrate improvement.

TABLE 4

| | Percent Improvement | Dowel Walking Time | | Grip Strength | |
|---|---|---|---|---|---|
| GROUP | Treatment | Time 1-2 | Time 2-3 | Time 1-2 | Time 2-3 |
| 1 | SHC1A-012 50 mg/kg | 11% | | 3% | |
| 2 | SHC1A-012 50 mg/kg | −26% | | 55% | |
| 3 | SHC1A-012 50 mg/kg | −13% | | 39% | |
| 4 | SHC1A-012 50 mg/kg - 2 | −33% | −55% | 90% | 256% |
| 5 | SHC1A-012 5 mg/kg - 2 | −3% | −40% | 10% | 11% |
| 6 | 50 mg/kg Scramble | −46% | | 0% | |
| 7 | None | −10% | | −7% | |

After the sacrifice times shown in Table 2 in this example nerves were extracted for quantitative PCR analysis to determine the total amount of human PMP22 mRNA present in the nerves. Effective SHC-012 performance should lower the amount of human PMP22 mRNA as compared to the scramble treated animals. The data from all animals is shown in Table 5. For percent reduction, the amount of PMP22 mRNA in the scramble group was determined and the percent reduction of each treatment group (compared to that scramble control) was calculated.

TABLE 5

Human PMP22 % Reduction

| GROUP | Treatment | Sacrifice Time | |
|---|---|---|---|
| 1 | SHC1A-012 50 mg/kg | 1 week | 61% |
| 2 | SHC1A-012 50 mg/kg | 2 months | 96% |
| 3 | SHC1A-012 50 mg/kg | 1 Month | 96% |
| 4 | SHC1A-012 50 mg/kg - 2 | 2 Months | 86% |
| 5 | SHC1A-012 5 mg/kg - 2 | 2 Months | 95% |

For the performance data shown above wild-type animals were also monitored to assess whether the different treatment days had any effect on the values. The numbers in Table 4 demonstrate a small effect which is likely simply due to testing variance (10% improvement in walking time but 7% detrimental results in grip strength).

Certain embodiments of the present disclosure can be defined in any of the following numbered paragraphs:

1. A composition comprising a compound that specifically targets a pre-mRNA to induce production of an exon-skipped mRNA via exon-skipping;
optionally, wherein the exon-skipped mRNA is detectable,
further optionally, wherein the exon-skipped mRNA is measurable above a background level.

2. The composition of paragraph 1, wherein the exon-skipped mRNA is a non-naturally occurring mRNA.

3. The composition of paragraph 1 or 2, wherein the compound reduces, but does not completely abolish, the amount of full-length mRNA expressed in a cell.

4. The composition of any one of paragraphs 1 to 3, wherein the exon-skipped mRNA encodes a non-stable and/or non-functional protein product wherein the codons downstream of the skipped exon are out of frame in comparison to the full-length mRNA.

5. The composition of any one of paragraphs 1 to 3, wherein the exon-skipped mRNA encodes a non-stable and/or non-functional protein product, wherein the codons downstream of the skipped exon remain in frame in comparison to the full-length mRNA.

6. The composition of any one of paragraphs 1 to 5, wherein the compound that specifically targets the pre-mRNA is an antisense oligonucleotide (ASO);
optionally, wherein the ASO is a PMO;
optionally, wherein at least one of the sugars in the nucleic acid backbone of the ASO is 2′-OMe-substituted;
optionally, wherein the ASO is conjugated to a delivery molecule to enhance cellular uptake, further optionally, wherein the delivery molecule enhances uptake by a specific cell type greater than other cell types, further optionally, wherein the delivery molecule is an antibody, peptide, a lipid, or a small molecule; and/or
optionally, wherein the ASO is formulated into a nano-particle to enhance uptake.

7. The composition of paragraph 6, wherein the ASO comprises or consists of a complementary region that is at least 70%, 75%, 80%, 85%, 90%, 95%, 96%, 97%, 98%, 99%, or 100% complementary to a target region of the pre-mRNA.

8. The composition of paragraph 6 or 7, wherein binding in a cell of the complementary region of the ASO to the target region of the pre-mRNA results in exon skipping of an exon during RNA transcription.

9. The composition of any one of paragraphs 1 to 8, wherein the target region of the pre-mRNA spans an intron/exon junction of one of the coding exons.

10. The composition of any one of c paragraphs 6 to 9, wherein the target region of the pre-mRNA spanning an intron/exon junction comprises at least 2, 3, 4, 5, 6, 7, 8, 9, 10, 11, or 12 nucleotides of the intron and a portion of the exon; or
wherein the target region of the pre-mRNA consists of 2, 3, 4, 5, 6, 7, 8, 9, 10, 11, or 12 nucleotides of the intron and a portion of the exon.

11. The composition of any one of paragraphs 6 to 10, wherein the target region of the pre-mRNA spanning an intron/exon junction comprises at least 2, 3, 4, 5, 6, 7, 8, 9, 10, 11, or 12 nucleotides of the exon and a portion of the intron; or
wherein the target region of the pre-RNA consists of 2, 3, 4, 5, 6, 7, 8, 9, 10, 11, or 12 nucleotides of the exon and a portion of the intron.

12. The composition of any one of paragraphs 1 to 11, wherein the target pre-mRNA of the compound is associated with a disease;
optionally, wherein the disease is a genetic disorder; or
optionally, wherein the disease is not a genetic disorder.

13. The composition of any one of paragraphs 1 to 12, wherein the composition is a therapeutic composition comprising a pharmaceutically acceptable carrier or diluent.

14. The composition of any one of paragraphs 1 to 13, wherein the compound is an ASO and wherein the ASO is a pharmaceutically acceptable salt.

15. A method of measuring the amount of an exon-skipped mRNA produced in response to an exon-skipping inducing compound; optionally, measuring the amount against an internal control, the method comprising:
(i) administering the composition of any of paragraphs 1 to 14 to a cell to induce exon-skipping of a target pre-mRNA and production of the exon-skipped mRNA,
optionally, wherein the exon-skipping inducing compound reduces, but does not completely abolish, the amount of full-length mRNA expressed from the target pre-mRNA;
(ii) obtaining a sample comprising the exon-skipped mRNA,
optionally, wherein the sample also comprises the full-length mRNA,
further optionally, wherein the sample comprises the cell;
(iii) measuring in the sample the amount of the exon-skipped mRNA; and
(iv) optionally, also measuring the amount of the full-length mRNA in the sample and comparing the amount of the exon-skipped mRNA to the amount of the full-length mRNA, optionally, wherein said cell is in a subject, the composition is administered to said subject, and the sample is a biological sample from said subject.

16. The method of paragraph 15, wherein the amount of the full-length mRNA is reduced and:
wherein the reduction in the amount of the full-length mRNA is not more than about 90%, 80%, 70%, 60%, 50%, 40%, 30%, 20%, or 10%;
wherein the reduction in the amount of full-length mRNA is between any of about 90%, 80%, 70%, 60%, 50%, 40%, 30%, or 20% to any of about 80%, 70%, 60%, 50%, 40%, 30%, 20%, or 10%; and/or
wherein the reduction in the amount of full-length mRNA in not more than about 50%.

17. A method of adjusting the dosing of an exon-skipping inducing compound, the method comprising:
(i) administering a dose of the composition of any of paragraphs 1 to 14 to a cell to induce exon-skipping of a target pre-mRNA and production of an exon-skipped mRNA therefrom, wherein the compound reduces, but does not completely abolish, the amount of full-length mRNA expressed from the target pre-mRNA;
(ii) obtaining a sample comprising the exon-skipped mRNA and the full-length mRNA, optionally, wherein the sample comprises the cell;
(iii) measuring in the sample the amount of the exon-skipped mRNA and the amount of the full-length mRNA;
(iv) determining the ratio between the amount of the exon-skipped mRNA and the amount of the full-length mRNA; and
(v) adjusting the dosing of the composition to be subsequently administered based on the ratio between the amount of the exon-skipped mRNA and the amount of the full-length mRNA, optionally, wherein the amount of the dose is adjusted and/or the frequency of administration of the dose is adjusted;
optionally, subsequently administering the composition to the same cell or to another cell according to said adjustment to the dosing based on the ratio between the amount of the exon-skipped mRNA and the amount of the full-length mRNA determined in step (iv); optionally, wherein said cell is in a subject, the composition is administered to said subject, and the sample is a biological sample from said subject.

18. The method of paragraphs 17:
wherein the subsequent dose to be administered and/or that is subsequently administered is adjusted to achieve a level of reduction of the full-length mRNA of not more than about 90%, 80%, 70%, 60%, 50%, 40%, 30%, 20%, or 10%;
wherein the subsequent dose to be administered and/or that is subsequently administered is adjusted to achieve a level of reduction of the full-length mRNA of between any of about 90%, 80%, 70%, 60%, 50%, 40%, 30%, or 20% to any of about 80%, 70%, 60%, 50%, 40%, 30%, 20%, or 10%; and/or
wherein the subsequent dose to be administered and/or that is subsequently administered is adjusted to achieve a ratio of the amount of exon-skipped mRNA to the amount of full-length mRNA of about 1:99, 2:98, 3:97, 4:96, 5:95, 10:90, 20:80, 25:75, 30:70, 40:60, 50:50, 60:40, 70:30, 75:25, 80:20, 90:10, 95:5, 96:4, 97:3, or 98:2 to any of about 2:98, 3:97, 4:96, 5:95, 10:90, 20:80, 25:75, 30:70, 40:60, 50:50, 60:40; 70:30, 75:25, 80:20, 90:10, 95:5, 96:4, 97:3, 98:2, or 99:1.

19. A method of treating a disease or medical condition with an exon-skipping inducing compound, the method comprising
(i) administering to a subject in need of treatment a dose of the composition of any of paragraphs 1 to 14 to induce exon-skipping of a target pre-mRNA and production of an exon-skipped mRNA therefrom, wherein the compound reduces, but does not abolish, the amount of full-length mRNA expressed from the target pre-mRNA;
(ii) obtaining a biological sample from the subject;
(iii) measuring in the sample the amount of the exon-skipped mRNA; and optionally (iv) measuring in the sample the amount of the full-length mRNA and determining the ratio between the amount of exon-skipped mRNA and the amount of full-length mRNA.

20. The method of paragraph 19, wherein the dosing of the composition is adjusted to be subsequently administered based on the ratio between the amount of the exon-skipped mRNA and the amount of the full-length mRNA and wherein the composition is subsequently administering to the same subject or to another subject according to said adjustment to the dosing based on the ratio between the amount of the exon-skipped mRNA and the amount of the full-length mRNA.

21. The method of paragraph 20:
wherein the subsequent dose to be administered and/or that is subsequently administered is adjusted to achieve a level of reduction of the full-length mRNA of not more than about 90%, 80%, 70%, 60%, 50%, 40%, 30%, 20%, or 10%;
wherein the subsequent dose to be administered and/or that is subsequently administered is adjusted to achieve a level of reduction of the full-length mRNA of between any of about 90%, 80%, 70%, 60%, 50%, 40%, 30%, or 20% to any of about 80%, 70%, 60%, 50%, 40%, 30%, 20%, or 10%; and/or
wherein the subsequent dose to be administered and/or that is subsequently administered is adjusted to achieve a ratio of the amount of exon-skipped mRNA to the amount of full-length mRNA of about 1:99, 2:98, 3:97, 4:96, 5:95, 10:90, 20:80, 25:75, 30:70, 40:60, 50:50, 60:40, 70:30, 75:25, 80:20, 90:10, 95:5, 96:4, 97:3, or 98:2 to any of about 2:98, 3:97, 4:96, 5:95, 10:90, 20:80, 25:75, 30:70, 40:60, 50:50, 60:40; 70:30, 75:25, 80:20, 90:10, 95:5, 96:4, 97:3, 98:2, or 99:1.

22. The method of any one of paragraphs 17 to 21, wherein the administration of the composition reduces the amount of full-length protein produced from a gene of the target pre-mRNA,
optionally, wherein the method further comprises measuring the amount of the protein;
optionally, wherein the subsequent dose to be administered and/or that is subsequently administered is adjusted to achieve a level of reduction of the full-length protein of not more than about 90%, 80%, 70%, 60%, 50%, 40%, 30%, 20%, or 10%; and/or
optionally, wherein the subsequent dose to be administered and/or that is subsequently administered is adjusted to achieve a level of reduction of the full-length protein of between any of about 90%, 80%, 70%, 60%, 50%, 40%, 30%, or 20% to any of about 80%, 70%, 60%, 50%, 40%, 30%, 20%, or 10%.

22. The method of any one of paragraphs 16 to 21, wherein the dosing of the composition administered is increased and/or is more frequent, thus increasing the amount of exon-skipped mRNA produced, decreasing the amount of full-length mRNA produced, increasing the ratio of exon-skipped mRNA/full-length mRNA and/or decreasing the amount of full-length protein produced,
optionally, wherein the dosing of the composition administered is increased and/or is more frequent, thus decreasing the amount of the protein produced from the gene of the target pre-mRNA.

23. The method of any one of paragraphs 16 to 21, wherein the dosing of the composition administered is decreased and/or is less frequent, thus decreasing the amount of exon-skipped mRNA produced, increasing the amount of full-length mRNA produced, decreasing the ratio of exon-skipped mRNA/full-length mRNA, and/or increasing the amount of full-length protein produced,
optionally, wherein the dosing of the composition administered is decreased, thus increasing the amount of the protein produced from the gene of the target pre-mRNA.

24. The method of any one of paragraphs 15 to 23,
wherein the biological sample is a cell, tissue, organ, or a sample obtained therefrom, or
wherein the biological sample is blood, plasma, cerebrospinal fluid (CSF), lymph, skin, saliva, mucus, feces, urine, eye fluid, saliva, stomach fluid, or a sample obtained therefrom.

25. The method of any one of paragraphs 15 to 24, wherein the amount of exon-skipped mRNA and/or the amount of full-length mRNA is measured using polymerase chain reaction (PCR), nucleic acid sequencing, oligonucleotide ELISA, and/or mass spectrometry.

26. The method of any one of paragraphs 15 to 25, wherein the administered composition comprises a pharmaceutically acceptable carrier or diluent.

The breadth and scope of the present disclosure should not be limited by any of the above-described exemplary embodiments, but should be defined only in accordance with the following claims and their equivalents.

### REFERENCES

Krajewski K M, Lewis R A, Fuerst D R, Turansky C, Hinderer S R, Garbem J, Kamholz J, Shy M E. Neurological dysfunction and axonal degeneration in Charcot-Marie-Tooth disease type 1A. Brain: a journal of neurology. 2000; 123 (Pt 7):1516-27. Epub 2000 Jun. 27. doi: 10.1093/brain/123.7.1516. PubMed PMID: 10869062.
Braathen G J. Genetic epidemiology of Charcot-Marie-Tooth disease. Acta Neurol Scand Suppl. 2012(193):iv-22. Epub 2012 Nov. 1. doi: 10.1111/ane.12013. PubMed PMID: 23106488.
Patel P I, Roa B B, Welcher A A, Schoener-Scott R, Trask B J, Pentao L, Snipes G J, Garcia C A, Francke U, Shooter E M, Lupski J R, Suter U. The gene for the peripheral myelin protein PMP-22 is a candidate for Charcot-Marie-Tooth disease type 1A. Nature genetics. 1992; 1(3):159-65. Epub 1992 Jun. 1. doi: 10.1038/ng0692-159. PubMed PMID: 1303228.
Timmerman V, Nelis E, Van Hul W, Nieuwenhuijsen B W, Chen K L, Wang S, Ben Othman K, Cullen B, Leach R J, Hanemann C O, et al. The peripheral myelin protein gene PMP-22 is contained within the Charcot-Marie-Tooth disease type 1A duplication. Nature genetics. 1992; 1(3):171-5. Epub 1992 Jun. 1. doi: 10.1038/ng0692-171. PubMed PMID: 1303230.
Valentijn L J, Bolhuis P A, Zorn I, Hoogendijk J E, van den Bosch N, Hensels G W, Stanton V P, Jr., Housman D E, Fischbeck K H, Ross D A, et al. The peripheral myelin gene PMP-22/GAS-3 is duplicated in Charcot-Marie-Tooth disease type 1A. Nature genetics. 1992; 1(3):166-70. Epub 1992 Jun. 11. doi: 10.1038/ng0692-166. PubMed PMID: 1303229.
Matsunami N, Smith B, Ballard L, Lensch M W, Robertson M, Albertsen H, Hanemann C O, Muller H W, Bird T D, White R, et al. Peripheral myelin protein-22 gene maps in the duplication in chromosome 17p11.2 associated with Charcot-Marie-Tooth 1A. Nature genetics. 1992; 1(3):176-9. Epub 1992 Jun. 1. doi: 10.1038/ng0692-176. PubMed PMID: 1303231.
Lee S, Amici S, Tavori H, Zeng W M, Freeland S, Fazio S, Notterpek L. PMP22 is critical for actin-mediated cellular functions and for establishing lipid rafts. The Journal of neuroscience: the official journal of the Society for Neuroscience. 2014; 34(48):16140-52. Epub 2014 Nov. 28. doi: 10.1523/JNEUROSCI.1908-14.2014. PubMed PMID: 25429154; PMCID: PMC4244477.
Mittendorf K F, Marinko J T, Hampton C M, Ke Z, Hadziselimovic A, Schlebach J P, Law C L, Li J, Wright E R, Sanders C R, Ohi M D. Peripheral myelin protein 22 alters membrane architecture. Sci Adv. 2017; 3(7):e1700220. Epub 2017 Jul. 12. doi: 10.1126/sciadv.1700220. PubMed PMID: 28695207; PMCID: PMC5498104.
Snipes G J, Suter U, Welcher A A, Shooter E M. Characterization of a novel peripheral nervous system myelin protein (PMP-22/SR13). The Journal of cell biology. 1992; 117(1):225-38. Epub 1992 Apr. 1. doi: 10.1083/jcb.117.1.225. PubMed PMID: 1556154; PMCID: PMC2289391.
Magyar J P, Martini R, Ruelicke T, Aguzzi A, Adlkofer K, Dembic Z, Zielasek J, Toyka K V, Suter U. Impaired differentiation of Schwann cells in transgenic mice with increased PMP22 gene dosage. The Journal of neuroscience: the official journal of the Society for Neuroscience. 1996; 16(17):5351-60. Epub 1996 Sep. 1. PubMed PMID: 8757248; PMCID: PMC6578876.
Sereda M, Griffiths I, Puhlhofer A, Stewart H, Rossner M J, Zimmerman F, Magyar J P, Schneider A, Hund E, Meinck H M, Suter U, Nave K A. A transgenic rat model of Charcot-Marie-Tooth disease. Neuron. 1996; 16(5):1049-60. Epub 1996 May 1. doi: 10.1016/s0896-6273(00)80128-2. PubMed PMID: 8630243.
Huxley C, Passage E, Manson A, Putzu G, Figarella-Branger D, Pellissier J F, Fontes M. Construction of a mouse model of Charcot-Marie-Tooth disease type 1A by pronuclear injection of human YAC DNA. Human molecular genetics. 1996; 5(5):563-9. Epub 1996 May 1. doi: 10.1093/hmg/5.5.563. PubMed PMID: 8733121.
Perea J, Robertson A, Tolmachova T, Muddle J, King R H, Ponsford S, Thomas P K, Huxley C. Induced myelination and demyelination in a conditional mouse model of Charcot-Marie-Tooth disease type 1A. Human molecular genetics. 2001; 10(10):1007-18. Epub 2001 May 2. doi: 10.1093/hmg/10.10.1007. PubMed PMID: 11331611.
Chance P F, Alderson M K, Leppig K A, Lensch M W, Matsunami N, Smith B, Swanson P D, Odelberg S J, Disteche C M, Bird T D. DNA deletion associated with hereditary neuropathy with liability to pressure palsies. Cell. 1993; 72(1):143-51. Epub 1993 Jan. 15. doi: 10.1016/0092-8674(93)90058-x. PubMed PMID: 8422677.
Zhao H T, Damle S, Ikeda-Lee K, Kuntz S, Li J, Mohan A, Kim A, Hung G, Scheideler M A, Scherer S S, Svaren J, Swayze E E, Kordasiewicz H B. PMP22 antisense oligonucleotides reverse Charcot-Marie-Tooth disease type 1A features in rodent models. The Journal of clinical investigation. 2018; 128(1):359-68. Epub 2017 Dec. 5. doi: 10.1172/JCI96499. PubMed PMID: 29202483; PMCID: PMC5749515.
Boutary, Suzan et al. “Squalenoyl siRNA PMP22 nanoparticles are effective in treating mouse models of Charcot-Marie-Tooth disease type 1 A.” Communications biology vol. 4,1 317. 9 Mar. 2021, doi:10.1038/s42003-021-01839-2.
Cornett, Kayla M D et al. “Natural history of Charcot-Marie-Tooth disease during childhood.” Annals of neurology vol. 82,3 (2017): 353-359. doi:10.1002/ana.25009 18. Cortese, Andrea et al. “Targeted next-generation sequencing panels in the diagnosis of Charcot-Marie-Tooth disease.” Neurology vol. 94,1 (2020): e51-e61. doi:10.1212/WNL.0000000000008672.
Eichinger, Katy et al. “The Charcot-Marie-Tooth Functional Outcome Measure (CMT FOM).” Neurology vol. 91,15 (2018): e1381-e1384. doi:10.1212/WNL.0000000000006323.
Gautier, Benoit et al. “AAV2/9-mediated silencing of PMP22 prevents the development of pathological features in a rat model of Charcot-Marie-Tooth disease 1 A.” Nature communications vol. 12,1 2356. 21 Apr. 2021, doi:10.1038/s41467-021-22593-3 21. Kagiava, A et al. “Intrathecal gene therapy in mouse models expressing CMT1X mutations.” Human molecular genetics vol. 27,8 (2018): 1460-1473. doi:10.1093/hmg/ddy056.
Lee, Ji-Su et al. “Targeted PMP22 TATA-box editing by CRISPR/Cas9 reduces demyelinating neuropathy of Charcot-Marie-Tooth disease type 1A in mice.” Nucleic acids research vol. 48,1 (2020): 130-140. doi:10.1093/nar/gkz1070.
Massade, Liliane, and Charbel Massaad. “Antisense RNA Targeting PMP22 for the Treatment of Charcot Marie-Tooth 1A Disease.” EPO Patent 18306241.3. 1 Apr. 2020.
Morelli, Kathryn H et al. “Allele-specific RNA interference prevents neuropathy in Charcot-Marie-Tooth disease type 2D mouse models.” The Journal of clinical investigation vol. 129,12 (2019): 5568-5583. doi:10.1172/JCI130600.
Reynolds, Angela et al. “Rational siRNA design for RNA interference.” Nature biotechnology vol. 22,3 (2004): 326-30. doi:10.1038/nbt936 26. Sandelius, Asa et al. “Plasma neurofilament light chain concentration in the inherited\peripheral neuropathies.” Neurology vol. 90,6 (2018): e518-e524. doi:10.1212/WNL.0000000000004932.
Schorling, Elisabeth et al. “Cost of illness in Charcot-Marie-Tooth neuropathy: Results from Germany.” Neurology vol. 92,17 (2019): e2027-e2037. doi:10.1212/WNL.0000000000007376
Stavrou, Marina et al. “Emerging Therapies for Charcot-Marie-Tooth Inherited Neuropathies.” International journal of molecular sciences vol. 22,11 6048. 3 Jun. 2021, doi:10.3390/ijms22116048.
Stavrou, Marina et al. “Genetic mechanisms of peripheral nerve disease.” Neuroscience letters vol. 742 (2021): 135357. doi:10.1016/j.neulet.2020.135357.
Tozza, S et al. “Motor performance deterioration accelerates after 50 years of age in Charcot-Marie-Tooth type 1A patients.” European journal of neurology vol. 25,2 (2018): 301-306. doi:10.1111/ene.13494.
Wang, Hongge et al. “MicroRNAs as Biomarkers of Charcot-Marie-Tooth Disease Type 1A.” Neurology vol. 97,5 (2021): e489-e500. doi:10.1212/WNL.0000000000012266.
Wang, Hongge et al. “Transmembrane protease serine 5: a novel Schwann cell plasma marker for CMT1A.” Annals of clinical and translational neurology vol. 7,1 (2020):69-82. doi:10.1002/acn3.50965.
Passage E, Norreel J C, Noack-Fraissignes P, Sanguedolce V, Pizant J, Thirion X, Robaglia-Schlupp A, Pellissier J F, Fontes M. Ascorbic acid treatment corrects the phenotype of a mouse model of Charcot-Marie-Tooth disease. Nat Med. 2004; 10(4):396-401. doi: 10.1038/nm1023. PubMed PMID: 15034573.
Gess B, Baets J, De Jonghe P, Reilly M M, Pareyson D, Young P. Ascorbic acid for the treatment of Charcot-Marie-Tooth disease. Cochrane Database Syst Rev. 2015(12):CD011952. Epub 2015 Dec. 15. doi: 10.1002/14651858.CD011952. PubMed PMID: 26662471; PMCID: PMC6823270.
Pareyson D, Reilly M M, Schenone A, Fabrizi G M, Cavallaro T, Santoro L, Vita G, Quattrone A, Padua L, Gemignani F, Visioli F, Laura M, Radice D, Calabrese D, Hughes R A, Solari A, Cmt T, groups C-T. Ascorbic acid in Charcot-Marie-Tooth disease type 1A (CMT-TRIAAL and CMT-TRAUK): a double-blind randomised trial. Lancet Neurol. 2011; 10(4):320-8. doi: 10.1016/S1474-4422(11)70025-4. PubMed PMID: 21393063; PMCID: PMC3154498.
Pareyson D, Schenone A, Fabrizi G M, Santoro L, Padua L, Quattrone A, Vita G, Gemignani F, Visioli F, Solari A, Group C-T. A multicenter, randomized, double-blind, placebo-controlled trial of long-term ascorbic acid treatment in Charcot-Marie-Tooth disease type 1A (CMT-TRIAAL): the study protocol [EudraCT no.: 2006-000032-27]. Pharmacological research: the official journal of the Italian Pharmacological Society. 2006; 54(6):436-41. Epub 2006 Oct. 13. doi: 10.1016/j.phrs.2006.09.001. PubMed PMID: 17029975.
Meyer zu Horste G, Prukop T, Liebetanz D, Mobius W, Nave K A, Sereda M W. Antiprogesterone therapy uncouples axonal loss from demyelination in a transgenic rat model of CMT1A neuropathy. Ann Neurol. 2007; 61(1):61-72. doi: 10.1002/ana.21026. PubMed PMID: 17262851.
Sereda M W, Meyer zu Horste G, Suter U, Uzma N, Nave K A. Therapeutic administration of progesterone antagonist in a model of Charcot-Marie-Tooth disease (CMT-1A). Nat Med. 2003; 9(12):1533-7. doi: 10.1038/nm957. PubMed PMID: 14608378.
Prukop T, Stenzel J, Wernick S, Kungl T, Mroczek M, Adam J, Ewers D, Nabirotchkin S, Nave K A, Hajj R, Cohen D, Sereda M W. Early short-term PXT3003 combinational therapy delays disease onset in a transgenic rat model of Charcot-Marie-Tooth disease 1A (CMT1A). PLoS One. 2019; 14(1):e0209752. doi: 10.1371/journal.pone.0209752. PubMed PMID: 30650121; PMCID: PMC6334894. [Pharnext, the employer of Serguei Nabirotchkin, Rodolphe Hajj and Daniel Cohen. DC, RH, MWS, TP and KAN submitted a patent based on this work: Full patent name: Early treatment of CMT disease Number: US2018/0000813. MWS, TP and KAN act as consultants to Pharnext. PXT3003 achieved a Phase 3 trial in CMT1A adult patients. There are no further patents related to this study, or marketed products to declare. This does not alter our adherence to all the PLOS ONE policies on sharing data and materials, as detailed online in the guide for authors.]
Sociali G, Visigalli D, Prukop T, Cervellini I, Mannino E, Venturi C, Bruzzone S, Sereda M W, Schenone A. Tolerability and efficacy study of P2X7 inhibition in experimental Charcot-Marie-Tooth type 1A (CMT1A) neuropathy. Neurobiol Dis. 2016; 95:145-57. doi: 10.1016/j.nbd.2016.07.017. PubMed PMID: 27431093.
Verhamme C, King R H, ten Asbroek A L, Muddle J R, Nourallah M, Wolterman R, Baas F, van Schaik I N. Myelin and axon pathology in a long-term study of PMP22-overexpressing mice. Journal of neuropathology and experimental neurology. 2011; 70(5):386-98. Epub 2011 Arp. 14. doi: 10.1097/NEN.0b013e318217eba0. PubMed PMID: 21487305.
Landis S C, Amara S G, Asadullah K, Austin C P, Blumenstein R, Bradley E W, Crystal R G, Darnell R B, Ferrante R J, Fillit H, Finkelstein R, Fisher M, Gendelman H E, Golub R M, Goudreau J L, Gross R A, Gubitz A K, Hesterlee S E, Howells D W, Huguenard J, Kelner K, Koroshetz W, Krainc D, Lazic S E, Levine M S, Macleod M R, McCall J M, Moxley R T, 3rd, Narasimhan K, Noble L J, Perrin S, Porter J D, Steward O, Unger E, Utz U, Silberberg S D. A call for transparent reporting to optimize the predictive value of preclinical research. Nature. 2012; 490(7419):187-91. Epub 2012 Oct. 13. doi: 10.1038/nature11556. PubMed PMID: 23060188; PMCID: 3511845.
FDA. Current Good Manufacturing Practice for Phase 1 Investigational Drugs. 2008.
FDA. CMC Regulatory Considerations for Oligonucleotide Drug Products: FDA Perspective. 2019.
FDA. IND Submissions for Individualized Antisense Oligonucleotide Drug Products: Administrative and Procedural Recommendations. 2020.
Fledrich R, Schlotter-Weigel B, Schnizer T J, Wichert S P, Stassart R M, Meyer zu Horste G, Klink A, Weiss B G, Haag U, Walter M C, Rautenstrauss B, Paulus W, Rossner M J, Sereda M W. A rat model of Charcot-Marie-Tooth disease 1A recapitulates disease variability and supplies biomarkers of axonal loss in patients. Brain: a journal of neurology. 2012; 135(Pt 1):72-87. Epub 2011 Dec. 23. doi: 10.1093/brain/awr322. PubMed PMID: 22189569.
Gautier B, Hajjar H, Soares S, Berthelot J, Deck M. Abbou S, Campbell G, Ceprian M, Gonzalez S, Fovet C M, Schutza V, Jouvenel A, Rivat C, Zerah M, Francois V, Le Guiner C, Aubourg P, Fledrich R, Tricaud N. AAV2/9-mediated silencing of PMP22 prevents the development of pathological features in a rat model of Charcot-Marie-Tooth disease 1 A. Nature communications. 2021; 12(1):2356. Epub 2021 Arp. 23. doi: 10.1038/s41467-021-22593-3. PubMed PMID: 33883545; PMCID: PMC8060274.
Duchesne M, Danigo A. Richard L, Vallat J M, Attarian S, Gonnaud P M, Lacour A, Pereon Y, Stojkovic T, Nave K A, Bertrand V, Nabirotchkin S, Cohen D, Demiot C, Magy L. Skin Biopsy Findings in Patients With CMT1A: Baseline Data From the CLN-PXT3003-01 Study Provide New Insights Into the Pathophysiology of the Disorder. Journal of neuropathology and experimental neurology. 2018; 77(4):274-81. doi: 10.1093/jnen/nly001. PubMed PMID: 29408953.
Svaren J, Moran J J, Wu X, Zuccarino R, Bacon C, Bai Y. Ramesh R, Gutmann L, Anderson D M, Pavelec D, Shy M E. Schwann cell transcript biomarkers for hereditary neuropathy skin biopsies. Annals of neurology. 2019; 85(6):887-98. Epub 2019 Apr. 22. doi: 10.1002/ana.25480. PubMed PMID: 30945774; PMCID: PMC7050273.
Wang H, Davison M, Wang K, Xia T H, Call K M, Luo J, Wu X, Zuccarino R, Bacha A, Bai Y, Gutmann L, Feely S M E, Grider T, Rossor A M, Reilly M M, Shy M E, Svaren J. MicroRNAs as Biomarkers of Charcot-Marie-Tooth Disease Type 1A. Neurology. 2021; 97(5):e489-e500. Epub 2021 May 24. doi: 10.1212/WNL.0000000000012266. PubMed PMID: 34031204; PMCID: PMC8356381.
Park N Y, Kwak G, Doo H M, Kim H J, Jang S Y, Lee Y I, Choi B O, Hong Y B. Farnesol Ameliorates Demyelinating Phenotype in a Cellular and Animal Model of Charcot-Marie-Tooth Disease Type 1A. Curr Issues Mol Biol. 2021; 43(3):2011-21. Epub 2021 Nov. 13. doi: 10.3390/cimb43030138. PubMed PMID: 34889893.
Morrow J M, Evans M R B, Grider T, Sinclair C D J, Thedens D. Shah S, Yousry T A, Hanna M G, Nopoulos P, Thornton J S, Shy M E, Reilly M M. Validation of MRC Centre MRI calf muscle fat fraction protocol as an outcome measure in CMT1A. Neurology. 2018; 91(12):e1125-e9. Epub 20180817. doi: 10.1212/WNL.0000000000006214. PubMed PMID: 30120135; PMCID: PMC6161551.
Scharner J, Qi S, Rigo F, Bennett C F, Kramer A R. Delivery of GalNAc-Conjugated Splice-Switching ASOs to Non-hepatic Cells through Ectopic Expression of Asialoglycoprotein Receptor. Mol Ther Nucleic Acids. 2019; 16:313-25. doi: 10.1016/j.omtn.2019.02.024. PubMed PMID: 30965276; PMCID: PMC6453860.
Brennan K M, Bai Y, Shy M E. Demyelinating CMT—what's known, what's new and what's in store? Neuroscience letters. 2015; 596:14-26. Epub 2015 Jan. 24. doi: 10.1016/j.neulet.2015.01.059. PubMed PMID: 25625223.
Morrow J M, Sinclair C D, Fischmann A, Machado P M, Reilly M M, Yousry T A, Thornton J S, Hanna M G. MRI biomarker assessment of neuromuscular disease progression: a prospective observational cohort study. Lancet neurology. 2016; 15(1):65-77. Epub 2015 Nov. 10. doi: 10.1016/S1474-4422(15)00242-2. PubMed PMID: 26549782; PMCID: PMC4672173.
Fridman V, Sillau S, Acsadi G, Bacon C, Dooley K, Burns J, Day J, Feely S, Finkel R S, Grider T, Gutmann L, Herrmann D N, Kirk C A, Knause S A, Laura M, Lewis R A, Li J, Lloyd T E, Moroni I, Muntoni F, Pagliano E, Pisciotta C, Piscosquito G, Ramchandren S, Saporta M, Sadjadi R, Shy R R, Siskind C E, Sumner C J, Walk D, Wilcox J, Yum S W, Zuchner S, Scherer S S, Pareyson D, Reilly M M, Shy M E, Inherited Neuropathies Consortium-Rare Diseases Clinical Research N. A longitudinal study of CMT1A using Rasch analysis based CMT neuropathy and examination scores. Neurology. 2020; 94(9):e884-e96. Epub 2020 Feb. 13. doi: 10.1212/WNL.0000000000009035. PubMed PMID: 32047073; PMCID: PMC7238948.

1-26. (canceled)

27. A composition comprising a compound that specifically targets a pre-mRNA to induce production of an exon-skipped mRNA via exon-skipping;
optionally, wherein the exon-skipped mRNA is detectable,
further optionally, wherein the exon-skipped mRNA is measurable above a background level.

28. The composition of claim 27, wherein the exon-skipped mRNA is a non-naturally occurring mRNA.

29. The composition of claim 27, wherein the compound reduces, but does not completely abolish, the amount of full-length mRNA expressed in a cell.

30. The composition of claim 27, wherein the exon-skipped mRNA encodes a non-stable and/or non-functional protein product wherein the codons downstream of the skipped exon are out of frame in comparison to the full-length mRNA.

31. The composition of claim 27, wherein the exon-skipped mRNA encodes a non-stable and/or non-functional protein product, wherein the codons downstream of the skipped exon remain in frame in comparison to the full-length mRNA.

32. The composition of claim 27, wherein the compound that specifically targets the pre-mRNA is an antisense oligonucleotide (ASO);
optionally, wherein the ASO is a PMO;
optionally, wherein at least one of the sugars in the nucleic acid backbone of the ASO is 2′-OMe-substituted;
optionally, wherein the ASO is conjugated to a delivery molecule to enhance cellular uptake, further optionally, wherein the delivery molecule enhances uptake by a specific cell type greater than other cell types, further optionally, wherein the delivery molecule is an antibody, peptide, a lipid, or a small molecule; and/or
optionally, wherein the ASO is formulated into a nano-particle to enhance uptake.

33. The composition of claim 32, wherein the ASO comprises or consists of a complementary region that is at least 70%, 75%, 80%, 85%, 90%, 95%, 96%, 97%, 98%, 99%, or 100% complementary to a target region of the pre-mRNA.

34. The composition of claim 32, wherein binding in a cell of the complementary region of the ASO to the target region of the pre-mRNA results in exon skipping of an exon during RNA transcription.

35. The composition of claim 27, wherein the target region of the pre-mRNA spans an intron/exon junction of one of the coding exons.

36. The composition of claim 32, wherein the target region of the pre-mRNA spanning an intron/exon junction comprises at least 2, 3, 4, 5, 6, 7, 8, 9, 10, 11, or 12 nucleotides of the intron and a portion of the exon; or
wherein the target region of the pre-mRNA consists of 2, 3, 4, 5, 6, 7, 8, 9, 10, 11, or 12 nucleotides of the intron and a portion of the exon.

37. The composition of claim 32, wherein the target region of the pre-mRNA spanning an intron/exon junction comprises at least 2, 3, 4, 5, 6, 7, 8, 9, 10, 11, or 12 nucleotides of the exon and a portion of the intron; or
wherein the target region of the pre-RNA consists of 2, 3, 4, 5, 6, 7, 8, 9, 10, 11, or 12 nucleotides of the exon and a portion of the intron.

38. The composition of claim 27, wherein the target pre-mRNA of the compound is associated with a disease;
optionally, wherein the disease is a genetic disorder; or
optionally, wherein the disease is not a genetic disorder.

39. The composition of claim 27, wherein the composition is a therapeutic composition comprising a pharmaceutically acceptable carrier or diluent.

40. The composition of claim 27, wherein the compound is an ASO and wherein the ASO is a pharmaceutically acceptable salt.

41. A method of measuring the amount of an exon-skipped mRNA produced in response to an exon-skipping inducing compound; optionally, measuring the amount against an internal control, the method comprising:
(i) administering the composition of claim 27 to a cell to induce exon-skipping of a target pre-mRNA and production of the exon-skipped mRNA,
optionally, wherein the exon-skipping inducing compound reduces, but does not completely abolish, the amount of full-length mRNA expressed from the target pre-mRNA;
(ii) obtaining a sample comprising the exon-skipped mRNA,
optionally, wherein the sample also comprises the full-length mRNA,
further optionally, wherein the sample comprises the cell;
(iii) measuring in the sample the amount of the exon-skipped mRNA; and
(iv) optionally, also measuring the amount of the full-length mRNA in the sample and comparing the amount of the exon-skipped mRNA to the amount of the full-length mRNA,
optionally, wherein said cell is in a subject, the composition is administered to said subject, and the sample is a biological sample from said subject.

42. The method of claim 41, wherein the amount of the full-length mRNA is reduced and:
wherein the reduction in the amount of the full-length mRNA is not more than about 90%, 80%, 70%, 60%, 50%, 40%, 30%, 20%, or 10%;
wherein the reduction in the amount of full-length mRNA is between any of about 90%, 80%, 70%, 60%, 50%, 40%, 30%, or 20% to any of about 80%, 70%, 60%, 50%, 40%, 30%, 20%, or 10%; and/or
wherein the reduction in the amount of full-length mRNA in not more than about 50%.

43. A method of adjusting the dosing of an exon-skipping inducing compound, the method comprising:
(i) administering a dose of the composition of claim 27 to a cell to induce exon-skipping of a target pre-mRNA and production of an exon-skipped mRNA therefrom, wherein the compound reduces, but does not completely abolish, the amount of full-length mRNA expressed from the target pre-mRNA;
(ii) obtaining a sample comprising the exon-skipped mRNA and the full-length mRNA, optionally, wherein the sample comprises the cell;
(iii) measuring in the sample the amount of the exon-skipped mRNA and the amount of the full-length mRNA;
(iv) determining the ratio between the amount of the exon-skipped mRNA and the amount of the full-length mRNA; and
(v) adjusting the dosing of the composition to be subsequently administered based on the ratio between the amount of the exon-skipped mRNA and the amount of the full-length mRNA, optionally, wherein the amount of the dose is adjusted and/or the frequency of administration of the dose is adjusted;
optionally, subsequently administering the composition to the same cell or to another cell according to said adjustment to the dosing based on the ratio between the amount of the exon-skipped mRNA and the amount of the full-length mRNA determined in step (iv);
optionally, wherein said cell is in a subject, the composition is administered to said subject, and the sample is a biological sample from said subject.

44. The method of claim 43:
wherein the subsequent dose to be administered and/or that is subsequently administered is adjusted to achieve a level of reduction of the full-length mRNA of not more than about 90%, 80%, 70%, 60%, 50%, 40%, 30%, 20%, or 10%;
wherein the subsequent dose to be administered and/or that is subsequently administered is adjusted to achieve a level of reduction of the full-length mRNA of between any of about 90%, 80%, 70%, 60%, 50%, 40%, 30%, or 20% to any of about 80%, 70%, 60%, 50%, 40%, 30%, 20%, or 10%; and/or
wherein the subsequent dose to be administered and/or that is subsequently administered is adjusted to achieve a ratio of the amount of exon-skipped mRNA to the amount of full-length mRNA of about 1:99, 2:98, 3:97, 4:96, 5:95, 10:90, 20:80, 25:75, 30:70, 40:60, 50:50, 60:40, 70:30, 75:25, 80:20, 90:10, 95:5, 96:4, 97:3, or 98:2 to any of about 2:98, 3:97, 4:96, 5:95, 10:90, 20:80, 25:75, 30:70, 40:60, 50:50, 60:40; 70:30, 75:25, 80:20, 90:10, 95:5, 96:4, 97:3, 98:2, or 99:1.

45. A method of treating a disease or medical condition with an exon-skipping inducing compound, the method comprising
(i) administering to a subject in need of treatment a dose of the composition of claim 27 to induce exon-skipping of a target pre-mRNA and production of an exon-skipped mRNA therefrom, wherein the compound reduces, but does not abolish, the amount of full-length mRNA expressed from the target pre-mRNA;
(ii) obtaining a biological sample from the subject;
(iii) measuring in the sample the amount of the exon-skipped mRNA; and
optionally (iv) measuring in the sample the amount of the full-length mRNA and determining the ratio between the amount of exon-skipped mRNA and the amount of full-length mRNA.

46. The method of claim 45, wherein the dosing of the composition is adjusted to be subsequently administered based on the ratio between the amount of the exon-skipped mRNA and the amount of the full-length mRNA and wherein the composition is subsequently administering to the same subject or to another subject according to said adjustment to the dosing based on the ratio between the amount of the exon-skipped mRNA and the amount of the full-length mRNA.

47. The method of claim 46:
wherein the subsequent dose to be administered and/or that is subsequently administered is adjusted to achieve a level of reduction of the full-length mRNA of not more than about 90%, 80%, 70%, 60%, 50%, 40%, 30%, 20%, or 10%;
wherein the subsequent dose to be administered and/or that is subsequently administered is adjusted to achieve a level of reduction of the full-length mRNA of between any of about 90%, 80%, 70%, 60%, 50%, 40%, 30%, or 20% to any of about 80%, 70%, 60%, 50%, 40%, 30%, 20%, or 10%; and/or
wherein the subsequent dose to be administered and/or that is subsequently administered is adjusted to achieve a ratio of the amount of exon-skipped mRNA to the amount of full-length mRNA of about 1:99, 2:98, 3:97, 4:96, 5:95, 10:90, 20:80, 25:75, 30:70, 40:60, 50:50, 60:40, 70:30, 75:25, 80:20, 90:10, 95:5, 96:4, 97:3, or 98:2 to any of about 2:98, 3:97, 4:96, 5:95, 10:90, 20:80, 25:75, 30:70, 40:60, 50:50, 60:40; 70:30, 75:25, 80:20, 90:10, 95:5, 96:4, 97:3, 98:2, or 99:1.

48. The method of claim 43, wherein the administration of the composition reduces the amount of full-length protein produced from a gene of the target pre-mRNA,
optionally, wherein the method further comprises measuring the amount of the protein;
optionally, wherein the subsequent dose to be administered and/or that is subsequently administered is adjusted to achieve a level of reduction of the full-length protein of not more than about 90%, 80%, 70%, 60%, 50%, 40%, 30%, 20%, or 10%; and/or
optionally, wherein the subsequent dose to be administered and/or that is subsequently administered is adjusted to achieve a level of reduction of the full-length protein of between any of about 90%, 80%, 70%, 60%, 50%, 40%, 30%, or 20% to any of about 80%, 70%, 60%, 50%, 40%, 30%, 20%, or 10%.

49. The method of claim 43, wherein the dosing of the composition administered is increased and/or is more frequent, thus increasing the amount of exon-skipped mRNA produced, decreasing the amount of full-length mRNA produced, increasing the ratio of exon-skipped mRNA/full-length mRNA and/or decreasing the amount of full-length protein produced,
optionally, wherein the dosing of the composition administered is increased and/or is more frequent, thus decreasing the amount of the protein produced from the gene of the target pre-mRNA.

50. The method of claim 43, wherein the dosing of the composition administered is decreased and/or is less frequent, thus decreasing the amount of exon-skipped mRNA produced, increasing the amount of full-length mRNA produced, decreasing the ratio of exon-skipped mRNA/full-length mRNA, and/or increasing the amount of full-length protein produced,
optionally, wherein the dosing of the composition administered is decreased, thus increasing the amount of the protein produced from the gene of the target pre-mRNA.

51. The method of claim 41,
wherein the biological sample is a cell, tissue, organ, or a sample obtained therefrom, or
wherein the biological sample is blood, plasma, cerebrospinal fluid (CSF), lymph, skin, saliva, mucus, feces, urine, eye fluid, saliva, stomach fluid, or a sample obtained therefrom.

52. The method of claim 41, wherein the amount of exon-skipped mRNA and/or the amount of full-length mRNA is measured using polymerase chain reaction (PCR), nucleic acid sequencing, oligonucleotide ELISA, and/or mass spectrometry.

53. The method of claim 41, wherein the administered composition comprises a pharmaceutically acceptable carrier or diluent.


SEQUENCE LISTING (239 sequences)

SEQ ID NO: 1 - DNA, 31748 bp, Homo sapiens
  source 1..31748: mol_type = genomic DNA
gttttgcctaggctaggaggagcccaatcccagaccatgctatccagtagagttacgggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggtaagtgatttggtggggagagtgaaggagatggaggagaaatgtgggcatcttccagtgagggtgccagaaagcgcagcgcaggcgcggggctttggccagctccctggggcttctgtttaggggcacagggtcccctgtgtgtcctgtttccctccaatgggtcttggagtaatacaacgaggagagcctttatggtctataagaccttacagggcaagggtgatggtgctggtgtctggatagcggataagggcggcccagttctcgccttgctgaagaggcgttgactctgggacacactggcaaaacagtccctgtggcggacatcccgagagtgttggactcctgtggttccccagttcccagcctcctgccatcggcttaattcaaaccctctgggagtcatcagaaatccttgtttatttcttccagtgcattccagagtttctttgaatgtgatgcttgtggagaggaacagaggggcctgggattgggtactttccaaactgggttattgaaacgttgtctccctggcccaagactctgcctaaaatggtccccagagtctcagccagatctttcacagccatgcatgtcctctaacaggtcatcccagccttcagttcccccaaagtttaaaatgaggggcgaggaccgcattacttgggataaatgtcctgccggctctgaccggctgaaattctcggactcagccttttaccctctgtctctctctcccgtccgctggacatctactcccttcggtccaggctcctgggctcctgtcccaccgcacccagaccggagctcaggcttgttggggttcgtgtcctggctttgagtgcctggggtgcagactggacccctaggcgagcacgtgggtctagcgcaagagcagaatgccccctgaccccaggggctgcctggggaagcgcgcggtggacgggaagcgcagggtccgggcaaccctctcgagccattcttttctttccacactactctggctctgcaccacctctccggagccgccagagtctgcgcgaggctgagctggggccaggaccgttcctctacgctggcagagttgggtggaaacttggagacaaacggaatgcggagagcactggggtctgggaaaccagcctcctcgccgctgtctcccccaccgcatgccacggctgtctccaggtctcagacggaaatctgggcacctactcccctcacccctacctccaccccataggggaattcaccatctgaaccggggtctcggagaacgtgacctctcaccttccagggaggtcgccgggaggtgcttggggtgggtggaagcgtgcagtggcctctgctcatattttctggaaacccctccgttccctgggtggttttagacgtgcgaaccgcttgttttgtttccaaaagcaaaagatgttccgttgcaggcgggcccggctgggcgctgggcactgggcgctggtcctgcaggcggctgctgccccctctcggcggcaggcggcgcgaaggctcctgacccgcgcgggcggtcgggctgcgggcgctgggccaggccgggccttccgctagtgcgcgggaccctccctctgcgcgcgcctccgtcgctcggcccagtgcgttcggcctcacgcccagcgctctcctcgcagatgctcctcctgttgctgagtatcatcgtcctccacgtcgcggtgctggtgctgctgttcgtctccacgatcgtcagcgtgagtgcctggcggggaggctccctgcgcggcccgcccttccccatctgggttcccagcccgtgctcctgctggttcaggactgtgttatttgcagacagttggaagtctcagacgtcccagggaagtttctggcaatctgcccccttccagttgctttgagaaaacgagagcagattcagtgataggaaccaagcgagcgctgggctgggttagctgcgcaggtctctatttaagccaagtaacttcagagccgatctagggtccccgaccttcatttgacaagattgtttaacttttttttttcttgatgcagcctcgttgaagaagaggagtatttatgatttttttttctccaaaccatagtcagatgggtgaattaatttataaaacacccttttaggacttgaaattggaaagttaaaatgggcttttctggaaaagagttcacaggtcccatgtcggctttatggcatgaacacaacacattgttagtaaagcttccactggtaggaacaggccatcagagtaacttctgttacaaaactgtccagcccattagatcttaaagttattttcttggcgatgaagatgagcagacattcaggcagttttccagtggtggctttcacttcttaatttaaggcacattgtaagcatgattttctttttcgttttcttttcttttcttttctttttttttttttttttgagatggagtctccagcctgtcacccgggctggagtgcagtgccatgatcttggcttactgcaacctctgccttccgggttcaagccattctcctgcctcagcctgggattacaggtgcccgccaccacgcctggctaattttttttttttttgtatttttagtagagacggggtttcactatgttggccaggcttgtctcaaactcctgacctcatgatccgcccacctctgcctcccaaagtgctgggattacaggtgtgagccaccgtgcccggcctgtaagcatgattttctagtctctgtgagaaataattatgttggggatttgtgacttagtttaacatttacaagcatcttcacagtatctccattgtgcctggtgatgatgatatcagactgataggagctataaacaaggaaatagactgagagaggcgggcttggggaatgatcccgagatgctgggaggagagaaggcttgaatgcaggtgtctaaggttgagttcatggctccttctactatagtaaacatctctgcacataatcgtttctgtgtgcatgtggaacttctccatttacaaggtgcttttaagtcataaaacgttggctcttaccatgcaggggtgggcggtgtggctaggtggatgcgggtgcttttcgccatccctgggcctttctccttccccttttccttcactcctccctccctccctgactcaggatatctatctgattctctctagcaatggatcgtgggcaatggacacgcaactgatctctggcagaactgtagcgcctcttcctcaggaaatgtccaccactgtttctcatcatcaccaaacggtgaggctggttttgtgctccatgagcttgtcctcagacgcctgtaacacgtttctcagcccagagctggaaacgcttattggaagacacaagccagaatgtcgttgctggggtggggtgggatgtgacagggagccatgagtccagggaaaatgggaggggtggtgatctcgctggggaagctgagatcctgggcatcattgtgattcactctacctagagacatgcccttagtgccctcctgatcacttaggataatgctttttgtgattgaaaaaaatatcgacctgggagctgtgaaggtggtatgatagctaagcctttggcagacccccttgggacattggatcaaacgtgtatctttggtttggtggctgtctctattcagaaatgtcctgcccctttctgctgcagtgacagtggggctagctcactgcaccgtcagttacatggacacaagactggctgcctttccaggtcttgggcctcagtgatgccacctagcatcaaaacctttgaatgtttccttgttgcaagatggggacttgaagctatttgcaaaaccctaagctaagccttggggtccttaatccagaagatcgtatttctcctctgtcctcagcatctttgcttgtgagatatggaagccaggcttcatgagggtcaaattaagggataattggcagcagaagcaccaactaggtacccaaggcacccagagggaaaggaacacccagccaggagccctgtgtgtttggatgcagacaggtggaggtaaacaggatgagctgttttggtggctgggtaggccatagcgattgatgttaagagcccgtggacctggatccacatgatcttttgttccagaagatgtgtcctcaggctggaaggacctagagatacagcagagctttcctaacatcctgggtttaaaaggccacggaaatacagctttaaatgttgctggagctactcatagatccagtatatatgttcaagatgctacaccagacgagagaaaccagtttagatccagttcagtaaaacaggcagtgaagtggcctttggtcctggctttgcataatccagttgctttgctggactttccctcttacagctcagtacccagtaatacccaagcatgtccccagctgaagcattataaaccaaattccttgaaccacagtacaaatagaaactgctaaaataataatttggtaatttacatggagaagatacgcaaggagtagaaaaccctgctttatgtttttaccttctgggttggggagggagcagagaaaaggtcccagtcctattggagtgagaacagtgcttaatcttgtctctgagtgtgtgaaaggcattgaattaaagccatcccagaaattacgtggtgggtttgcatggtgagttggcaactgagtaaatgaaatactcatgagtgcctgagaatgtgaccgtagcactgcttgtccccaaagccacagcatccggtttctccccctttagggtctggctgaagttcccggagcactccggatgggagtctccgtactcgctactgacacctggtggttacaagtagtgactcagcccgaaaagggcaggctttggtgctcacaggcgccatccccaagtggcatctggccacgcggcttggcacagaatcctggactcttgggcccggaagggggcagttttggcaggcgtgttaaaacccggcgaagtttcagcagaaacgaggcggcagaggagttgctaagttgtgcttaaaccatctgcaggaaagaaagcaatattgacctgtagcatgttacagattgacatttggtgtcttctgccgttggaaatagctgtcagttggtgctagaagcagatctcaaagaggcactagggttattttaatcaggaaacttcttatctctcaacttgttccttctgtgccaagctcatgttcttgagttcatttacaatgcctccttagtgtgggtttcaaatctgcctcgttgctttccgtgaggaccccagagtgtctgttttccacatctcccttctcagctctcacaatcagggcattttgaaaacatctggacactgcacctgaacatgctgggtttgttttcacgccactcaggcttaatctaaatttgaaatttccatttacacttccccagtagtggttatctctgccgctatcttccccaacgtggcagcacttgctacgacttcagccttaagtggcgaatcctccagggcctctttgattgagtttaacaattgtggctgcagacaataagctgaaaaaaatgtgtactttttaaaacattatactgtctttacaaatgaactatgctcattgcgaaccatttaacttgtctgtatttgctcttagaaagtatatttgaaaaaaaatacttgttaagtaatcaaaagtaatttgttattgacatcgatttgtgaagaatatgttgcaagtgtgaaaaaataaatagtggtgcaatctcggctgactacaacctccacctcctgggttcaagcgattttcctgcctcagcctcccgagtagctgggattataggcgcccaacaccacgcctggctaatttttgtatttttagtagagaggggtttcatcatgttgggcaggctggtctcgaactcctgacctcaggagatccacccacctcacctcccaaagtgctgggattagaggtgtgagccactgcgccatgtgacttggtatttttttctgaaaggactcatttttctggtgcatgaccatgacacttcacctctctctgggggttgccactcatgttgttttctacatgaatggggcctcctggcatcgtgccatgaggcagtctaacatctaggttgtgtagtttgaaagagacacaccagatcttgcccccaggctacatcttaatattagaaatagttttagatttttaactaactgactttgactccgcctgccttctcgtttttctaaatgtgtttgtatactttctggcaccccctccttatctgcctcgtgtgggcagaggtaagagaggcttagtgtggggatattcgttatgatattgtatccagtgcctctccggcccagggaatacaaggatctggaatgcatgggccttgccttgatggtcccgagcctggtcgaagagtcaggagatagatatggaagggtgaatcactcccttggaaggcagagcaggccaggtgcccaaagggaggcaggcagacagcgtctgagttcagggaggaagcggatgggaaatctctggtgggaggtagaatctgatccatcctccaaaggatgggcaggatttaagtcaatacaggcaacggggaggagaatattccctgtgacctaaggtaagggtagaacaaggcaggggatggggtagactggttggctagtgttctggttaggattaggattgattgtgtataagagaacatcttcaaatgataggggcttaagtaagatcatttatttttctctcttttcaaagaagtctggaagtaggtagcctgagagaggtatggaggttccatgaaattgtcagagatgcagacactttctagctcatttctttgccatctcttgggtttggctgtcatcttcaggaaacaaaaggctgctagagctccagccatctcatccatgattcaagcagtagggtggagaaaggaggtggggaagaatgaatctccttttcttttaatgaaatgtgacatggtcacacctacacctatttacaagagaggctgagaaatatagtcattcggccttgggggaagcatggcttttcaaatcatagaaacctggcagcaaatacctctctgtaagcctggtgtggaatactttccttgagggtttgaccatcccctcccatacccaatcatactcttttgcaatagctcagttgggccctatgcagttccataagtcccagttatttcagcattcttcaaagagtccatctctgctgcatcctgggctctgtcagccaccccaccattctcacatgggctcttctcagaaactttaggacctgttgaggcttctctctctctatttctaccacctctccaggtataagtaagtcttattattattattgagacagagtttcactcttgttgcccaggctggagttcagtggcgccatcttggctcactgcaacctccgcttcccgggttcaagcaattctcctgcctcagcctcccgggtagctgggattacaggcatgcgccaccacgcttggctaattttttgtatttttagtagagacgggctttctccatgttggtcaggctggtctcgaactcccgacctcaggtgatccacctgcctcagcctcccaaagtgctgagattacaagcgtgagccaccatgcctggccatcttattattatttaatgagtccagacctctcttctgaatcccagacctgtttatccagctgtctcctggccacttcccttgggatatctaagagaggcccccaagtcactaggccagagacagtccttatcttcctctgccaattcaacccctctgtcactttctagtattttagtgaatgctgcttcaccagccagcaagccaaacatctagagtctgccttgacttgacgcgtttccttctttctcttctcagcattgtcacgggtccctcaggccaccccattgttccaatgcctgttgcccacctgtggtcacgctgcttcctcgtttgtcagaggcgccagggcagcctcctaactagtttgcctgaaccctttcttgctcctctgtgactcatttttcatatggaatccaggctgatatttaaaaaagcacaaacctaattatatcactcctcattttgaaacctttcagaggccgttcattggtcttaatcttcagatgaaaatctttaacaaggcccatcggaatctacctgaatgatccactcctgcttctgttaagtctcacctcccatctctcttcccctcatcttggaatttcagcgttttctcacgtcctgaaatgtgaacttcccacttcgggagtctctccatttctcttctgatctttcatgcaggactatttcttgaacaactctcgtgtaccagagactattgcaggtgctaaggttacaagagtgaacaagagcttatgttttactggacaaaccagtcagtaaacatggagaatttgggggcaatggtaagaactatataagacagggtaatgggataaagagggattgcagtggtgaaggaaggtctccttgaggagtggccaggagccagccatatgaacatctcggggaggttccatacagaaggaccgtcaagtgcaagatcgctggggagggaaggagcttggcgtagttgagggacagagggcagggcagactagtgggaacagagagtgaggggtaagtagaatcacagaccatctcaaaggtaccccccacccccactaggaaggagttgggtgcacatggatgcatttctaatctttgtgatttccttagacctgctgtcttccacgggtttctcagtggtccatttgattccaagttggagtgacagtccagaagcctctataaggcagaatatatcagaaattgtacaaggtagaaatgcatcggtatattttcagtgattattttgggtgacgggattcttatcactttggctgttttctttcttttgctgccttacctgttttcctcattgagatcttttattttcacaatcagaaaataactgtgaacattttatgttagaaaattggttttgagtagtaagtaggttccctctgccttgattaagagagtggagccctggggcatggcacgcagggtccatggcgtcgctttgaatgaggccagttcctggagctttagtttgcaaactcatacaatctggaaggctctggtgagggggaggggtgtgccaagcctcctccccaagaccctatgggtgagtttaaaatctactgacccaaaggattcaggcaaaacatttgccctgctattgaatgaaccagtgctttttgttttgaagtctccaagatccaaatatcaagtccaagttctcacaagcattcccctgctttttgtttaatttgtgtggacctgacgtgtagcagaatgggtcagaatactggaattaagccacaggcggcttgggagcccaagagctagaagggcacagtcctggcttgccctggcccctggctggtcttcatcagcttcagtgatgaggcaggacccaggcagtttgtcttcagtaaaatctgtctctcagggcctgtccctgctcacaaagtcctctcttgtctcagaagaaaaggtagttgataaactttttcatgctgaggtgttaggcacaaaaaatgtgcccctttttgaatcacttttgtttttaaaaaggaggccagtgctcaacaccaaagcatgaagggaattgttgagctttcctgtaatctgtccccaaacgtccttggccacctccattgtctaagggcagtacctgcagcccctcatttcccagtgacaacaggtgggtctgctctgccacagtgtgtgggcgggaccagcaccagacgctgagaagatgaccacttactactcttcttttccatttaaacagcagcagcagcagcagcagcagcagcagcagcagcaacagcaaactcatgactatcaataggttgtgtgaggaattagatcaatgctttgggttgaaaattagagaaactaaatcttgccagtctcagcagcccccaacctgagcccttggtggtctcctatcatcgctgtcttcaaaacgatcccagacttgtttatcaatttggtgacactgtcattttggcaaggacaccagacaatttccttgtctgtggctcagcttttggccagattttagaaaacacgggagaccttctgcatttatgggttcatgttttacattttaaaatcttcgtggctccctcagccaacccataatctctacccaaggaatggctctggattttacaggctcttatgagatgctgagaccctggtggtacaagggaggtacagaggacggaatgtgttgtcaacatgccaggatttcaacccttagggtttctctgatgccaatacccaggaattagtatgaaatgtttgtttgaacctgtgtctgaactgtatgacctggaatctatagtctttgctttaaaaaaacttcaattgtatccccaagatttcaccagcaccaagaaaacatcccttgcatttcaggagcagggactggccatggtgcagcctgaggttgtggatgttgatctttggtgcccgagagaaggctgtctgggatgaagccttctgactgtggtcaggtccctgccagtgctgggggccacatgagtgtgcagtcatccacacacaagtggcccccaacactggcagggacccggagtcatctggcccatccctccttcctatcccatcgagccacagacccctctgcaatatgcctaccacatgttcatcctaccactgatggggaacttattaccttcaagagagcctcttttgtttttgaagagctaatagtgagatccattcatgtgttcttcaactctattgagcctgtcctatgtactgggcactgttctagaaatggggtgcacagagatgaacaagaaagagcaggtccctgccattgtgcagctcacaaggagttggaggaaacaggcaatacacaatacatgcatcactatgggtagtggaacatgctctgcaggaaacaaatagggtgatccaatggaaagtcatcactggaggaatgagctgctcagggaaggcctcttccttccatagtgatgtggtctgagatttagaagacagggaaggaccagttataggaagaaccatgggaagaaaattctaggaggtaaagggaagaaccaaggccctgagttagctgaagaaaggagggtggggaatgcgggagagagggatgggagcaggatcgctggtgcctcacaggctgtggtactgagaaggacagttgggacatcattgaagggttttaagcagaagagtgtcatgatttgattcatgttttgtaagctcattctggctgttgagaatgaattctggggagaggcaagagtggaaactggggaccagtgaggagtttgtggtcatagtccagactggacatgatgatgacactgagatgacaatgatgacaatgacaatgatgttaagaatacaaacagctcacgtaagtgccaggcattctttgaaaagcaatgcatttaatcctcacaagaactctaggaggtagctggtggcttagctagggaggggaagtagagaagaaaacaggtctcatgacagggtttgctgatgggttagatgtagggagttagggaagaatgattctagatgactctacgttttgggcttgagcaactggtgtgtaactgaatcttggactgagatggggagactggaggaggaacagaagatttgcgggaaggtgtagaaatcaggatttatattttgaccatcagagaggtcagcaaatcacttcaatttagaagtctttggcttaggggagaggtctgggctggaaattcaaagtcaagagtcattcctctataccgaagggtattcaatgttgtggtgctgaatacagttacttggggaggagttgagagcagaaggcagaaaaggggaacagggttcagagctaagtcttgggcatgccaggggttaacgagggtacaggaaggagaggagactggggatgaacagccagtgatgagggaggaatgccaggagatattgtcacttaagaaattacctcggctgggcgcggtggctcacgcctataatcccagcactttgggaggctgaggcgggcggattacaaggtcaggagattgagaccatcctggctaacacggtgaaaccctgtctctactaaaaatacaaaacaaaattagccgggcgtggtggccggtgcctgtagtcccagctactcgggaggctgaggcaggagaatggcgtgaacccgggaggcggagcttgcagtgagccgagatcgcaccactgcacgccagcctgagcgacagagtgagactccgtctcaaaaaaagaaattaccttgttaaacccaacactgcccctcttttgtccattcacttctagttgccttcattcatgcctgatgtgctggacttgggttttatcaagttgcttggcacgtccttaaagaatttaaatctccacagtgcctcctggacaatgactgcagggtgacccccgtctctcctctacacgatgactcttcaaacagttgacgattgcagtttttctttccttccttctttcagtggcttctctggtgctggtcccagctgcataagcagggcctcttcttgcccttctgaagtttggtcagtggtttggcttggcaggctgatgagcagagaataagggaagccgctctctctttgccacaccattctccttccaccctctctagtttttgtggattctaagtctgaagaatggtggtagaataatttaattcattagattgcaagcatatcattgacttctctgcacccccagtgtgttgtagtggaaaaactagtagtctgggcatcagggaatccttgtttttctgcaccgggtctgcctgtaacaaattaggtaacctggggcaggtaccctggtttcctaaaagcagagcaggcattgaggtaggacctgaagttgaggtaggacctgcacattgcatgggtcccggagcctctgttcatgttcaggatgtaattgtggcatcgagggatcctaaaaggaggtggcctctgagctgggccttggcaatggggtagaattgcagtaggcagagggagcagagatgggttttgtgcttgtaaacgggcattggtttactgtcactggtgggggtagcagctcttttgttctagctcttcatttccataatgcgtgttcttttttcatactttaggggaaagaaggaggagaactctattatttctatggggagaattcctcctaaacctgaagatcttaaaactaagcgaattattccctgttcattctccactgatgcagttcatgactgcaattgcaactgcctttcccgtcattttctatggccaaactcagtgtttttaagtgagctcttcttttaaaaaacaaaaacaaaaacaagtctcctgataaatccgtttgaaagacactaagttttgaaagttaagcaggctttgatcattcatggtacattgtgaattaccaaggagggagattgatatccttcatcgtacaatgcactcccctcctttttcttttgtatctggtggagtaagtcttccaagagcaattcttagagaaacaaagcctactctgtttccctgtttctagagttttccaacaagggtataaaaaagcctcagacagcttactattatccagtaacctcaggtacctcagtggctgcgtgttattccctttagaagtccaaaaactcactagcagagcaagaatatggatgtcaaagtgcagaactagaccctgacaaactagctctgtgcctgccacaaagcctcttaattaggaatgcagttcattgaataactgtatcaaagttagctggaataccttgacatgaagattccttccacttactgagcagtttgtgcccactagtggccagacacaggctttgctctagctaaaatacccccaggattacctgttgagagagccgcccacgtcaccttcatcgcctttgtgagctccatgctggcacatagttgtctccatctgttttgctttccgcatgaatcataggcaaagttagcctgaccagcaagcccatttcaaagccaccagctgggggagaagttgaagcccagggggcaagacccacgctgggctttgggcatgtttgagctggtgggcgaagcatatgggcaaaggccacattgtttaggatggaggcctttcagagactcagctatttctggaatgacattcatactgagaaataaggaaaatggcgatctgtgtgatggttgtgggttgggaggtttgggcgtgggagtcctggtcttggggtcatgtgttttgaaaacagtctagcactatgcaaatgggaggtgttaataactctttgcctctgtgatttaccctctcatggctttttctctcttggcctctccaaggtctcttctgactcttgcaactgtccttcctttcactttcaacaaaacctgttctcttgagtcagaacagttttatgaattgcccagagtgggacttgatatggggcagggttggttccatgctggtaatgaaagatgcacataaactgttacattgaaaagtgcttcatactttctgagcccttaactcactaggttgtaggccctgggctaagaatatagctgagggggctgatgtatttccttcattgctcagctgctcctacagcaggaatctgaccttgaaatgagctttgatctttgtgcaactcgagcttcccactatctgctgggggagctaggtctgggtgctgccacggcccatgtcagaaagctgtcagcagctcttataccggacactctcagaacaggaggcctgcaaagagctttagctttagttttgttgttgttgttgtttttgagacagtctcgctctgtcgcccaggctggagtgcagtggcgccatctcggctcactgcaagctccacctcccgggttcacaccattctcctgcctcagcctcccaagtagttgggactacaggtgcccgccaccacgcccagctaattttttgtatttttagtagagacgaggtttcaccgtgttagccaggatggtctcaatctcctgacctcgtgatccacccgccttggcctcccaaagtgctgggattacaggtgtgagccacggcgcccggccttttttttttttttttttttgagacgagtcttgctctgtttccaggctggagtgcagtggtgtgatctcggctcactgcagtctccgccttccgggttcaagtgattctcctgctccagcctcccaaatagctgggattacaggcacatgccaccacacccagctaatttttgtatttttagtagagacggggtttcaccatgttggccagtatgatctcgatctcctgaccttgtgatccacccgctttggcctcccaaagtgctgggattacaggcatgagccaccacgcccggccagagctttagtttttaattccagtgctcagcaccaaggttggatattggcttaaaacttccactggaaactccacaaatgtttagcaggcctccactttgcaccaggaactgagtgtggcctggaggacagcaggtactcaacataagttctactatttattgcatacttgcaaattgggtggggcgtggggaagagtatcgccacactggggcttgtgttaatctatagtttagccacttgggatgttggtgtcacactaggtttgatctaaatagaacaggtttgtaattgacagcattttcacttgcattatatttctctgaactcgaatcattattttctgtacagctgacttgttttgctgtagcagagtttggcagataatagcttgcatgtcgaatccaacctgcttttgtaaataagattttattggataaatggctattgaaggaatgggtcacacccactcatttacatcttgtccatggctatggctgattttgtgatacaaaggcagaattgagtagttgttaagaagaccgtatgacctgcaaagcctaaagtatttgctctctgattctttatagatagtttgctatctgtgcagatatttgcactctgactctgtagcacccttatgctggagaggagatatgaattgataccaaagttgtatcaaaacacttttggagttctggccaatgtgctaataacctggcttgtgtgaacattgtggctctcattttcttttttcttttttttttttttgagacgagtcttgctctgtcgcccaggctggagtgcagtggcgcagtggcgcgatctcggctcactgcaagctccgcctccagggttcacgccattctcctgcctcagcctccagagtagctgggactacaggcgcccgccaccacgcccggctaatttttttttttttgtatttttagtagagacgggttttcactgtgttagccagcatggtctcgatctcctgacctcgtgatctgccctcctgggcctcccaagtgctgggattacaggcgtgacccactgcgcccggcctgtggctcccattttcatactgcttggtgtgacccagatggaaatgctccagaacctggcccctcaataacagcctgtaaatgcttgtttaccctgttttctcacatcagccttttaggtggggttatttgcactatcctttaacagcgcttcaataacaactcttttgtttgatcattatgacaacccaataaaatatattagtgcctcaagtacgtaaagcagaattcaaggccaaaaaaatgtatgtggcaagttaggtgggcggtcagaagaacttggcaacagagcccagccctgcctgggctgtcctaaccagagcacataaatgctggagagagtcaggctttgcacgtgctcacacgggacccttaccttggacaggatcaaagccctgtcatttctggagtgtgtgatatctgctgtctcttactgcgctggttgttacctgtctattccagttctgatgaatggctctgattggggagcagcatcatctgagcttagaaatgtcctcagggccctgtggggttccctttgagcaacaacgagagctcttgaatcttgcaccagccttttggagctggaatggagtttgctttctcctgggctgggctctgttttttttcctctcgctgtccttgtgtagcttttgatcaatggcactggaggaaaggaaaatcccttgttgttttcctgggcatgtggaagcccagtcctgcccagaattccactagccctttgagttttggaatcctgactcttagtaagtcaatagagtgctgcatctgtttttacctttgtaatcttttaaattggttttaattatttaagtaatacaagtattataatacatgaaaatatcctcaactgaaaggcttcttgaccaaactcttcttcgttgcaacagtagttcctgggggaacccactgacatcaatttgacacaactttggctctttctctacgaatatttgcatgcctgtggatgtctatacgcaattacatagttttttggtgtttgttttcataatgatgacatgttgtgtgtgcaccttactcttttcattgtcttggagacctttcaaagactcttcattttctaactcatgatttttttaactgctgcaaaatatttcattgtgaggatataccataatttattttccctcatagaaatgctacaatcaatgtccttatttattttcctctgagtatatgttcactgtttttccaggatatagtagataagcacttgaacacatatagtgtcattacgctagaggtgttacaatttttaaactacttttaatggcaaaaattacaattacttttgtatcaacctaatatattctgccacaaaatggctggtcacaaaaggattataataataatattattattattcctgtgaatggtgtctgcaggtagctatcacttatttgttctgctgatattatccattttttttgaattttgcctattggctggatgaaaattggtgtcaccctcctcatgggtttctcttgcatttccttggttgctagtgaggttgggcaccttttacgatgtttattggctgttcatatttctcctttgctaatcacatggcttttcaattttatacttagctgttttctctttgttctgtctgtaatttaattttgttaatgatgcgaactagagatttaattttatgttttaccaaatgaatagccaacactatgtattgtctgttgtatttttccccactggtttatatgatgttgtgtattatctaccaaatttacatgtatgcttccaaagtcttattccatcccattcttttatttttctttcatgattaacaccatatttttgaattgctaaaactttataatataaatataagctttgtatgtctataaataaaatacattttattaattataattatatgttatataattataatataagcacttgaacatatatacttctataattatatattatataattatatatcatacttctatgatatgttctataattatatattgtatatatcatagttctatgatatgttctataattatatattataaagttacaattaattgtatatttaatttataccatatactgtaataagcaaaatatagaaatatgttttaaatctggtaggtggtatcttgactcattggttttatatgtttttctgattttctttttgttcatttgttcgttttggtttttagttgctgttgtgtttacttcaaattttttcttatctgtcattgctcattgttttttagatgaatttttaattgatttttttccagttccagttgcaatatggattaggacagcattgaatttgtagattattttggatgaaattggcatctttataactttgagtgtttctatccaggaacttagtatgtttctttagtacttcttttatgtacttgaataaaatttgtctaacttgttgatgtatccccaaagaaccagacatttttattaaatgtattttaatatttttttctgtctcattagtctgtgcttttatccatattaagtcaattaatttttggacttatttcattgttttttttctggcttcttgagttgaaagtgtagttcactctttttaaggatttttctagcaaatgcttagaagtgtatacattgtcctctgagaactgctttggttacaacccacaggtgtgccatgtggctctctatataattccttgctgaagaagtttttacttttcatcttgttttttttaatccgaggaattatctggaacactgtttttaaatttccaaatttattttggctagtttgtttttaatgtctaatttctgtttgcctttaggtatatgttgagcctacatcatttctgcttctatatttattgagatattctttgttgcccttatagtcagtttgaaaagctgtcccatgtgtgtttgaaaataactttgctatatagacacaagttttctatacatctattaggacagatttgttaatcctcttatgtaatctatattctttcttctttttggtctacctgacatctattttgaggaaggaagctaaagtcttctactgttattgtgattttaacaatttcttctcatattttgattactttttgattttaactttttcaggccatgttattatatgcataatgatttattttcttcagtcatagatcatgtaaaccagaagtaaagatcaaataaaccaaatctttcctctatgaaacaaccaaacaactctctggccacttttatcctttcaacataagttctattttttattattaatttatcttctttcttttttgttaacatttgcctgaagcttcttttcagaatcttttttgtttttgactgttgcttgttttaggtgtatctattatattagaatatagctacaatgtctctcttttttttttttttttttttttttgagacagagtctctctctgtgcccaggctggagtgcagtggtgtgatcttggctcactacaacctccacctcctgagttcaagcgattctctgcctcaacctcccaagtagctgggactataggtgtccaccagcacacctggccaatttttttgtatttttcgtagagatggggttttggcatgttggccaggctggtcttgaactcctggcctcaagtgatctacctgcctcagcctcccaaaatgtcttaatcaaagataaaattccatatcttatgagtgccttgaacccattcacatataatgtggttgatgatagacttactatcttaatgcatgtgttttatttattgtgcttatggttgaaaaccatcttcttcatctctggatcatcagagtcaaccagagggcttggctcaaagcagtaggtattcaacaaatgctcattgaataaaaatgtccttgatcctctaattctgatttgattcgagaaataacttatggcaaagagctcaattgtaatggggttgggactaaaattcaagttaatgttgaaagatgtttcaagaagtatttaggttatattattaaatattagacactttctgtttatatagatttaacatataaagattttaagttttctgtgtatgtgaggtctcgtagtagtttctgctgaaaatgacaggggcgcggggttaagggtgaagacagtatctcaggattggagaaggaaatctaagatattgtctccaccagccatactcctcatgaacatgcacatggaaggacatttcccagccaaagtgaatccctttatttgttttagtttcagatttgagtctcagatttctttgtaattccagctgcatctaccaaagtgctagataatttttttattttaagcaaacaatattatgagaacattatctttattgcaaagtgctcctccagaagaacctctatgctggataaagacaaacatttgattaaggttatgtaagagtgaaatcaggatggctcacaacccattagccgtcattttcttactttaattgacaaagaaattgaaaaaatgaaagaagcatatgttgtgcttgaatgtgaagagtgctagactagaatcaaaggcctcttagatttggctgtgtcatttgggctcatgaaaacacctctggaaatctctttcagtgcaaaggagctggactagttgatctctcaggtttcttccaactctaaaatatctccagtctgtgccttggaaacatcttaggtgaaaatctaggaacagttaacctaatttgcacccttaaaattctgccatgagctgcttacaactcaaaacaagtttatcttactcagttactaattataaaccatccagatttcagagctgtgagtactgggtgaagcattgaaggtatgcttttgaagccattacatatggcagttactgagctgaaaggattaaatgctgcagcttccccagttgcccttcctccatgagagcagtgcctgcccccagcattctgtggcacttggaagacaagacagaggccaaatgcagatttttaccctgggcttccctctacagtgtggaactcaggttgtttcttctttcctccctgaaatgacatgagtttgcagcggatggtgaactgaagaaaccataggaggctctgtcttcttgcctgaatttcagttggaagcttggagatttggggttcaacagagataaggaagtgtaagccttcatcccgtctggtggttggcgatcacacaccgctctgtgctgaggctaatggccatgatcagagttgaccaaaaaaaaaaaaaaaaaaaaatacgggttgtccaagcaaattgatttccatacctatagagagcatacctttcttcatcagtattttcttccattcttccaaaaaattactttgggctctaacagccattcccgtgatctttacctctccttggggaatgcagataatttgaatagtggttttaagctatttttcttggaatacagaagttctgataagcccttcaaagacccctgagggcaagaagagggaaggtagtaggcagggctcaggccctctttaacacagacacatgtacataagtaacacatttgcaccaacactggaaggaaattcaatatattttaaaatgactttaactcctagtgatggaactatagatatttttttacagcccttccctgaatgttcacatgtgttttttatatatatgtgtgtgtcacgtatttgtatatgtgtatatacacatatatatagaaacacgtacatatttgtatgcatatttgtacatataggtatatatgtatgtaatgtgtacatattggcacatgtgtgtgtttgtatatgtatatatcagtacatatagatgtatgtatatatgtatatattttttcctccaaaataaacaataaagaatgtcttcttagattgcaaaagtgaattgtctgagttctgctaaggaaaaaggacttctgcttctgctgcctgtgaggactgtgctgtcagcagtcatggcccttcaggccctgcacctcgaggcagagccctcccccggccatggccagctctcctaaccaagtgtctctttcccccagaatggctgcagtctgtccaggccaccatgatcctgtcgatcatcttcagcattctgtctctgttcctgttcttctgccaactcttcaccctcaccaaggggggcaggttttacatactggaatcttccaaattcttgctggtaagttgtggatggtaaagtccatgtggaagcggggtgcatccaagtctgcggaatgattagtttagtagaaggatgtggcctcagaatgactgatgttcatgagtctccccactggatgctttccataaagtgagggtgggtgcttgtatgtgtgggtgtgtacctgtatgtgtcttagaacttgggacttagaactctccccttctccctggaatgagatgcatatgaaagagaacttagaggatctggaaggaaggtccccacccaagccaggcgtatcaacaggaatgaaactgcaatctggacacataatcagaggtgaatactgaggctatctgtagagcaaaggtcaggcttgagagctgtttctgtagattacattatgcctccagaaaatggccctgatgtgctaagaactagcaaagtagttatcaggtatgtgtcttccaccaataggtagtgatgaagccacactgacaaattctcaccttccttgcttccagttcctagattctactgggctttgattgactgttgtcatcctctggtgtcttcattttgacactcttgtgtcacatattgtcatttccaaacatggggctatgacaacacatgaaaacacatgagaggtctccttaatctcctgcctaaactgtcttcaagttccctctttaaatacgttattaatatgcatagtgtgcagagtcctagaaacttctgttacacagggtgacatcttccaactttgtctctggattctgcctagcatcttacatgcttacatcttacatcttacatcttacatcttgcattctgcctagcatcttacattagctcttacatgtctgtctgttgacttactgttgactgaaccagcagggcattggagagaagtaagagctagatgtagtggtggattctgtggtccaaattcatagatcacaaacttcatatgtaccagagtatgtctaggtactgggagatgttctcaattctgaccctctgagagggcaaaggatgtagcatctcttctctgagttggttgtcagaatgcccatggtaccatttcaccactctgtccccaggagcagtcattggaaggttgacgtaaatagggttgtatgggaagacacagcccaaggttagatgttggtgaccttgtctagaagacagagagttcccctttcctgaaaaaaggaagtaaatgattaaccacttctcattaaacactcaaatacaacatttcaatactcatggttttgagatttcaaaaccagacagtgctttgctacttacacatgtcttatgacaccaagccaagctcctggatggttgctggctctgttaaatgactaattatgcaaggagatatcatttctaggtacgttaaagtgaagagttacccttactcaattttcagttggaataaaaacaactgtaacatattctggggtttctttttttttttctcactcgttttagtttgatatcaaatcaaataatgatcatatccattgcatcagtggatatgccctcaagataatatggatttagaaccagaactttcataatgtatttctattgaaatgttagtttcataagcgatgattgggttttcatgcccatgtgtgagatgtgcctcgctcaaaccttgttatgatttggcacgttacccatctgatgtgaaaaaaattacattttatttgtacaggctcgttattttactgatgaataatttgagcccaccagaggataaatgaatgaccaaggtcacccagctcatgacagggacggttgagtgttacactgaatatagtgaggtacttcttatattttaaagacagaatgcaccaaaaaatttaaagaacacaaaatccaaggcagaagctctgccttttatattatcttttattggaactgatttacaatggaaggtaaatgcaaatttgcaccatgtattattctgaagttccaaacatctgtgatgaatacaagcctgtactataagacccagtcacattgaaaatatggagctgagaagaggtaagctgctgttgaatgggctccttgggatagccagtaccttcatcttcattcatcctgctgagctgtttcggctttaagttctttaacaatgtctttttagcaacatcattacatcattttaggccaaaactcaaagtccagagataagaacccttaagtcactcatgtaactgcactgtgtgttaaaagtatttcagttcagccaaacactcttctcctaggtattgcgatttaagtatatttactaatccactcttgcttcactattttcattctcctccaaagtcaatacaagatgtttagaactgtgctggaagtgcagaattccgaatgtaaaagcgcatgactttgtcctctttatcccctttacatctagctgcttacgtctcatgaaactgaattttcagttatctgttggtccacatttgaataagaaattatcttgaatttgaaatgctgagctgtaatagcagttgtaatttgtaagtcctgagagtgtcctgtcctccgttgttaatcccagtcaagatcatctgagagttggtctccagggaacctcagatctctaggatgttgcactggaaatggctgcaggatctttccactaattctgagaactgaaagagttaggaactctatttggagagttcctggttcccttatcgtgtgacagttctaagtcaattttgtcatgtggttttctgactcacagactagtaagaagttagtaattagagagctaagtagattagggttgttggagatgagaaacccacgttttgggaaaacctggcaagtgacaacttaacatcaaggaagtagtcagaaaagctaaaactgacaaacagaaggtagaagaaagtagctccacactcatgggatgtgaaatctacaagcgtgcatgcccggcaaatgcctctccaatgcactggagcgttttaagtggaaaccactagaatctctgtgtagtctccggaagtggctgtgagggctgcattatctctgcacagcttcctcttggtgggcccagctgtgatctttatggatggcacacatcagctttcaggaaaagcacatgaaaggtgctagggctcttggagctgactgtaggtttgggagttgtctgtctccttgctctagattacagctctgtgtgttgtgtggggtctccatggtttgccaaattatctcttcctcacttagccacaaggctgacagttaggaactatctcttcttgattgcattaggttggctgcttcctgaatgcatatcaaaaggctccttcctttagttcagtgctttcaacctgagctgtgcatcagaatcacctgaggtcctttaaaaaaaaaaaaaagacagtgggcggggcgcagtggctcacgcctgtaatcccagcactttgggaggctgaggtgggcagatcacttgaggtcaggagttcaagaccagcccggccaacatggtgaaaccctgtctctactaaaaataaaaataaaaaaatagccaggcgtggtggtgcgtgcctgtagtcccagctacttgggaaactgaggcagagaagaatcacttgaacctaggaggtggaggttgcagtgagccaagatcatgccactgcactccagcctgggggtgacagagtaagactgtctcaaaaaaaaagaaaaaagaaaagaaaaaagacagtattcaggtctcatccctggagactctcatttaatagttgtgggatggatccactgcctaggtgactctgatgtgcatttagggttgggaaccactgacatagccattaaactgtccctaatcccactgtaaggttttctaggatattttcccagaaataactaaaccaccttcttagagaaggaacatcctgatcctgcgtctggactttggagtcattcttattttcagaacctatagccatctattccttgacaagatctgttgggttgggttgcactagactggaaaacatcaagaaattaatctagacacagacctaaaaggaagatttgcacgttttgatttattttactgcttacacccagcatcaagctccatgtggggcacatagaggagcctgaataaaactattattggttggatgattagataattgtgtttcctcggcagaatcaaactcaagtcaaatgtgtggttaccgggattaagaacagaaagaatagaggggactcctgagtgagtttcactttctctctctttttgttattgttgttcatttgttttgttaatagaaagatcaatatagctatagagcatttaaaataaaaggatttgggccaggtgtggtggcgaatgcctgtaatcccaacactttgaaagactgaggtgagaggattgcttaaagccaggagttcaagaccagcctgggcaacaaagcgagatgccatctctacaaaaaatgatttaaaaattagtggggtgccttggagcacgcctgtggtcccagctattcaggaggctgaggcaggaggattgcttgaggctgagaggtcaagactacagtgaactatgatcaggcccctgcacttgctccagcctggtgacagagtgagaccctgcctctctaggaaaaaaaaaaaaagaatttggtggggggggattaataaaagttcagtgccatcctgttcagagtgattcaaaggtggcttaagtcaaataccacatttagaaatttttgttagggacggatgattatggatgtctcactggccatgcccaaatcagagtcaatgctatgggtggctttctaggcagcaatggtcatttggatactgaaaatgtaaaaggagtgggcttcaatcacagaaacacagagaagtctcttgctttcagaggaacaggcctcacagccccttcctgccctcccttgttgccctgaggatgaagtgggagggagaaaaaggcacccactcatcaacccaacatcatagctgcaccttccagtgaccaccctggtggtctcactgacatccctttccagccaacttctctctgacttctgccccaggctctcaaatgaaagtgcttttgcgaggttaccaaagtacactttgaagtctttaatttgttggatcactctgctcctttcaatactgttagctacttactctttgaagctctattcccctggcttctgagacatcgctgtgttcccacttgcctgagtgaacacaccaggcggtcatgctggcctctcctctctccctcaccccacagccagccttctactgaatcctgcatttgggtagcatcctccgtaggcattacgcccatttctttgtcttgactcactaaccccagtgcagctatgcaaaggcctcctcatgcaccttccagctacctgccttgtccctgcccctgaatccattttctgcaccatggccagagtgatcattctaaaacgtgcgtctgatcatgtttctcctctgctgaagtttcctcagaggcttctcatcacagcttccttaacatgtcatgtaaggccctctctttcctgacccttagtgagctaatcagcctattatatgagagtccgtgtggcttctccagttctccagtttggactcagcgcccgtctatgctcttggggcatctggtgcatttccttccctctctcgaaattccatcgaactatattgaaattatatgcaaatgtctgtcttctagacacacagtccttaaggtaggagccatgtctcacttatcttggtacatctagagcagcagctagaacagggcccgcatgcaataggcactcattaaatgttcactgaaccaaagggaatggagatgataaggatgtactgggaattccctcagctacttcctttgaccttggccccttgggtgcttccttccagaggctctgggctctaatcactctcatggggttgcagtcactgttaaaggagcttaagactttctcttcaaaagggctttaggcagaacttgcaggaagttgttgcagacctacccatcctgagacagggagaaactcttgtaagttgaatgctcagcacatttgtattgtctggacagggtcagtgtccttctgcttaaagatgacctatgctcccagactcaggcccctaccagggagtcccggtcattgccaaagagcagaacatctgcctgtgcctcagggcctcacttacccttgccacagggacctggtgatacatctccacctactggttgctgagaaagtgagtcacagtccacttactaggggtatttggcttttggagatgactactggatgacattaactgtcttgggttgcagacagagggaacccagccaaagaatcatccttttctctatttcagggtacatctattgctttacatgcagacaatcttgtaataatatattccctaaaacatcagtctcatgacaacaatcatataaagtgtgtatcctctctttggctttgtagatatctaagtttactgcttatcacggttaagcttagagtcattcacttctgagattctactaagatgaaagtcatacacattaagctgtgtagttagtcgctgcacttctctatactgcctctttctatccctcttgttcaaggatggaatctggtatctcctcttagtcagtgaaatcggggagtagctggtccactcagctcctgaggcatttctcatgccagatctggttagacagcccatttgggagccctgcctgcatgatgataagtgttgtcctgccctgctagcactctgttaacacctttctctctgccaaggtcatgtttccttttttatagccctgccttcattagataagtcagacagagcccaagagacaacttcagtatttcttaaagacaagagttagcactttggagatgcaagccgtggaagagtggggcaagataattaaggatcctggacctcaaggcaaataaagtgtgtccccagcagatcaaaactgctgttttctgcaatcaacaattaacccgcaatgatttcgctcctgacatggccatctttgctgtcttctgaacttcctgtaggtggcaggtcctggatgctgtcccattttggttccaagactattgtgtggctctcttgacaggggccagtcaagtggcagttttctcctctgagttgcaggaggacaggcttaacagggtggttcatgtggcgtattcccacttctgcactgtcttcaaacaaagttgcagtgtccagtaaaacagcagcccaaatagcagtgaggttagatgtaggtggccgggcggtgggggggcgcgggtgcattgggaactctggaactgcatgaatcccacacggcatactggatttataatagagtcactcagagcttctcaaagaccctgttttcagggaggaaacagaccaggctttgtgcagctccaggctggggtgctgacatcactggacccctccttgtggggggacgctgtctgtcacgtgagctggggccatcagtttgagcgtggctgctccacagggcccaagatggaggcttttcttctctttccaacaccgaagggctgcattggtgggccagggagggtggcctgacccacctgcatatccagtgccctttagccttctttgggcttcaaatgcaaccttggggcacccagggaaatggagcttgcctttgatcatcttccccctctgcagcgtggcttcccagtcatactgtcggctgcatccttattcctcactttctttttccctctccacacatttctttgtctgtttggcagtgcccataggcattgcttgcttcacctggtatgtggcaactctgttagggggaccataggcccctgtggtctgcatggagcttaatccttttaccatggcgtgactccaataatggtaaaatgtcacttcacttccattggctttaggtaatgccaccactcacttgtagagtggcatatagatttccaggggtttccatacccaaccacccttggaaagagacaggtaggtttttcttgcagcctgaatgcctgctggccatgtagtgtggcctgggaattggtgacagtgaaagaaagagaaatagggcagaaaatgtaaagaaaaccctcaggtattaaggcctaggtggccaagattggaaatagagagcaagttgttttccttgttcccatatttttgtgctacctcctttcaattctggacctggaagcagatttccttccagaggtagaaatgttcttcctacccagcaattgtcagcatccggggtggcggagagggggccgctctgccatggactctccgtcacccagcttgtccctctccttccccaggtctgtgcgtgatgagtgctgcggccatctacacggtgaggcacccggagtggcatctcaactcggattatcctacggtttcgcctacatcctggcctgggtggccttccccctggcccttctcagcggtgtcatctatgtgatcttgcggaaacgcgaatgatctcttgagtttttgtgggaacttgggtgtgggtacaaggtgcctgttgg

SEQ ID NO: 2 - DNA, 58 bp, Homo sapiens
  source 1..58: mol_type = genomic DNA
tcaggatatctatctgattctctctagcaatggatcgtgggcaatggacacgcaactg

SEQ ID NO: 3 - DNA, 25 bp, Homo sapiens
  source 1..25: mol_type = other DNA
agtcctatagatagactaagagaga

SEQ ID NO: 4 - DNA, 25 bp, Homo sapiens
  source 1..25: mol_type = other DNA
gtcctatagatagactaagagagat

SEQ ID NO: 5 - DNA, 25 bp, Homo sapiens
  source 1..25: mol_type = other DNA
tcctatagatagactaagagagatc

SEQ ID NO: 6 - DNA, 25 bp, Homo sapiens
  source 1..25: mol_type = other DNA
cctatagatagactaagagagatcg

SEQ ID NO: 7 - DNA, 25 bp, Homo sapiens
  source 1..25: mol_type = other DNA
ctatagatagactaagagagatcgt

SEQ ID NO: 8 - DNA, 25 bp, Homo sapiens
  source 1..25: mol_type = other DNA
tatagatagactaagagagatcgtt

SEQ ID NO: 9 - DNA, 25 bp, Homo sapiens
  source 1..25: mol_type = other DNA
atagatagactaagagagatcgtta

SEQ ID NO: 10 - DNA, 26 bp, Homo sapiens
  source 1..26: mol_type = other DNA
tagatagactaagagagatcgttacc

SEQ ID NO: 11 - DNA, 26 bp, Homo sapiens
  source 1..26: mol_type = other DNA
agatagactaagagagatcgttacct

SEQ ID NO: 12 - DNA, 26 bp, Homo sapiens
  source 1..26: mol_type = other DNA
gatagactaagagagatcgttaccta

SEQ ID NO: 13 - DNA, 26 bp, Homo sapiens
  source 1..26: mol_type = other DNA
atagactaagagagatcgttacctag

SEQ ID NO: 14 - DNA, 27 bp, Homo sapiens
  source 1..27: mol_type = other DNA
tagactaagagagatcgttacctagca

SEQ ID NO: 15 - DNA, 27 bp, Homo sapiens
  source 1..27: mol_type = other DNA
agactaagagagatcgttacctagcac

SEQ ID NO: 16 - DNA, 27 bp, Homo sapiens
  source 1..27: mol_type = other DNA
gactaagagagatcgttacctagcacc

SEQ ID NO: 17 - DNA, 27 bp, Homo sapiens
  source 1..27: mol_type = other DNA
actaagagagatcgttacctagcaccc

SEQ ID NO: 18 - DNA, 27 bp, Homo sapiens
  source 1..27: mol_type = other DNA
ctaagagagatcgttacctagcacccg

SEQ ID NO: 19 - DNA, 27 bp, Homo sapiens
  source 1..27: mol_type = other DNA
taagagagatcgttacctagcacccgt

SEQ ID NO: 20 - DNA, 27 bp, Homo sapiens
  source 1..27: mol_type = other DNA
aagagagatcgttacctagcacccgtt

SEQ ID NO: 21 - DNA, 27 bp, Homo sapiens
  source 1..27: mol_type = other DNA
agagagatcgttacctagcacccgtta

SEQ ID NO: 22 - DNA, 27 bp, Homo sapiens
  source 1..27: mol_type = other DNA
gagagatcgttacctagcacccgttac

SEQ ID NO: 23 - DNA, 27 bp, Homo sapiens
  source 1..27: mol_type = other DNA
agagatcgttacctagcacccgttacc

SEQ ID NO: 24 - DNA, 27 bp, Homo sapiens
  source 1..27: mol_type = other DNA
gagatcgttacctagcacccgttacct

SEQ ID NO: 25 - DNA, 27 bp, Homo sapiens
  source 1..27: mol_type = other DNA
agatcgttacctagcacccgttacctg

SEQ ID NO: 26 - DNA, 27 bp, Homo sapiens
  source 1..27: mol_type = other DNA
gatcgttacctagcacccgttacctgt

SEQ ID NO: 27 - DNA, 27 bp, Homo sapiens
  source 1..27: mol_type = other DNA
atcgttacctagcacccgttacctgtg

SEQ ID NO: 28 - DNA, 27 bp, Homo sapiens
  source 1..27: mol_type = other DNA
tcgttacctagcacccgttacctgtgc

SEQ ID NO: 29 - DNA, 27 bp, Homo sapiens
  source 1..27: mol_type = other DNA
cgttacctagcacccgttacctgtgcg

SEQ ID NO: 30 - DNA, 27 bp, Homo sapiens
  source 1..27: mol_type = other DNA
gttacctagcacccgttacctgtgcgt

SEQ ID NO: 31 - DNA, 27 bp, Homo sapiens
  source 1..27: mol_type = other DNA
ttacctagcacccgttacctgtgcgtt

SEQ ID NO: 32 - DNA, 27 bp, Homo sapiens
  source 1..27: mol_type = other DNA
tacctagcacccgttacctgtgcgttg

SEQ ID NO: 33 - DNA, 27 bp, Homo sapiens
  source 1..27: mol_type = other DNA
acctagcacccgttacctgtgcgttga

SEQ ID NO: 34 - DNA, 27 bp, Homo sapiens
  source 1..27: mol_type = other DNA
cctagcacccgttacctgtgcgttgac

SEQ ID NO: 35 - DNA, 58 bp, Homo sapiens
  source 1..58: mol_type = genomic DNA
gtccaccactgtttctcatcatcaccaaacggtgaggctggttttgtgctccatgagc

SEQ ID NO: 36 - DNA, 58 bp, Homo sapiens
  source 1..58: mol_type = other DNA
caggtggtgacaaagagtagtagtggtttgccactccgaccaaaacacgaggtactcg

SEQ ID NO: 37 - DNA, 25 bp, Homo sapiens
  source 1..25: mol_type = other DNA
caggtggtgacaaagagtagtagtg

SEQ ID NO: 38 - DNA, 25 bp, Homo sapiens
  source 1..25: mol_type = other DNA
aggtggtgacaaagagtagtagtgg

SEQ ID NO: 39 - DNA, 25 bp, Homo sapiens
  source 1..25: mol_type = other DNA
ggtggtgacaaagagtagtagtggt

SEQ ID NO: 40 - DNA, 25 bp, Homo sapiens
  source 1..25: mol_type = other DNA
gtggtgacaaagagtagtagtggtt

SEQ ID NO: 41 - DNA, 25 bp, Homo sapiens
  source 1..25: mol_type = other DNA
tggtgacaaagagtagtagtggttt

SEQ ID NO: 42 - DNA, 25 bp, Homo sapiens
  source 1..25: mol_type = other DNA
ggtgacaaagagtagtagtggtttg

SEQ ID NO: 43 - DNA, 25 bp, Homo sapiens
  source 1..25: mol_type = other DNA
gtgacaaagagtagtagtggtttgc

SEQ ID NO: 44 - DNA, 25 bp, Homo sapiens
  source 1..25: mol_type = other DNA
tgacaaagagtagtagtggtttgcc

SEQ ID NO: 45 - DNA, 25 bp, Homo sapiens
  source 1..25: mol_type = other DNA
gacaaagagtagtagtggtttgcca

SEQ ID NO: 46 - DNA, 25 bp, Homo sapiens
  source 1..25: mol_type = other DNA
acaaagagtagtagtggtttgccac

SEQ ID NO: 47 - DNA, 25 bp, Homo sapiens
  source 1..25: mol_type = other DNA
caaagagtagtagtggtttgccact

SEQ ID NO: 48 - DNA, 25 bp, Homo sapiens
  source 1..25: mol_type = other DNA
aaagagtagtagtggtttgccactc

SEQ ID NO: 49 - DNA, 25 bp, Homo sapiens
  source 1..25: mol_type = other DNA
aagagtagtagtggtttgccactcc

SEQ ID NO: 50 - DNA, 25 bp, Homo sapiens
  source 1..25: mol_type = other DNA
agagtagtagtggtttgccactccg

SEQ ID NO: 51 - DNA, 25 bp, Homo sapiens
  source 1..25: mol_type = other DNA
gagtagtagtggtttgccactccga

SEQ ID NO: 52 - DNA, 25 bp, Homo sapiens
  source 1..25: mol_type = other DNA
agtagtagtggtttgccactccgac

SEQ ID NO: 53 - DNA, 25 bp, Homo sapiens
  source 1..25: mol_type = other DNA
gtagtagtggtttgccactccgacc

SEQ ID NO: 54 - DNA, 25 bp, Homo sapiens
  source 1..25: mol_type = other DNA
tagtagtggtttgccactccgacca

SEQ ID NO: 55 - DNA, 25 bp, Homo sapiens
  source 1..25: mol_type = other DNA
agtagtggtttgccactccgaccaa

SEQ ID NO: 56 - DNA, 25 bp, Homo sapiens
  source 1..25: mol_type = other DNA
gtagtggtttgccactccgaccaaa

SEQ ID NO: 57 - DNA, 25 bp, Homo sapiens
  source 1..25: mol_type = other DNA
tagtggtttgccactccgaccaaaa

SEQ ID NO: 58 - DNA, 25 bp, Homo sapiens
  source 1..25: mol_type = other DNA
agtggtttgccactccgaccaaaac

SEQ ID NO: 59 - DNA, 25 bp, Homo sapiens
  source 1..25: mol_type = other DNA
gtggtttgccactccgaccaaaaca

SEQ ID NO: 60 - DNA, 25 bp, Homo sapiens
  source 1..25: mol_type = other DNA
tggtttgccactccgaccaaaacac

SEQ ID NO: 61 - DNA, 25 bp, Homo sapiens
  source 1..25: mol_type = other DNA
gctttgccactccgaccaaaacacg

SEQ ID NO: 62 - DNA, 25 bp, Homo sapiens
  source 1..25: mol_type = other DNA
gtttgccactccgaccaaaacacga

SEQ ID NO: 63 - DNA, 25 bp, Homo sapiens
  source 1..25: mol_type = other DNA
tttgccactccgaccaaaacacgag

SEQ ID NO: 64 - DNA, 25 bp, Homo sapiens
  source 1..25: mol_type = other DNA
ttgccactccgaccaaaacacgagg

SEQ ID NO: 65 - DNA, 25 bp, Homo sapiens
  source 1..25: mol_type = other DNA
tgccactccgaccaaaacacgaggt

SEQ ID NO: 66 - DNA, 25 bp, Homo sapiens
  source 1..25: mol_type = other DNA
gccactccgaccaaaacacgaggta

SEQ ID NO: 67 - DNA, 25 bp, Homo sapiens
  source 1..25: mol_type = other DNA
ccactccgaccaaaacacgaggtac

SEQ ID NO: 68 - DNA, 25 bp, Homo sapiens
  source 1..25: mol_type = other DNA
cactccgaccaaaacacgaggtact

SEQ ID NO: 69 - DNA, 25 bp, Homo sapiens
  source 1..25: mol_type = other DNA
actccgaccaaaacacgaggtactc

SEQ ID NO: 70 - DNA, 25 bp, Homo sapiens
  source 1..25: mol_type = other DNA
ctccgaccaaaacacgaggtactcg

SEQ ID NO: 71 - DNA, 25 bp, Homo sapiens
  source 1..25: mol_type = other DNA
atagatagactaagagagatcgtta

SEQ ID NO: 72 - DNA, 24 bp, Homo sapiens
  source 1..24: mol_type = other DNA
ctaagagagatcgttacctagcac

SEQ ID NO: 73 - DNA, 25 bp, Homo sapiens
  source 1..25: mol_type = other DNA
atcgttacctagcacccgttacctg

SEQ ID NO: 74 - DNA, 21 bp, Homo sapiens
  source 1..21: mol_type = other DNA
tagtggtttgccactccgacc

SEQ ID NO: 75 - DNA, 20 bp, Homo sapiens
  source 1..20: mol_type = other DNA
gccactccgaccaaaacacg

SEQ ID NO: 76 - DNA, 58 bp, Homo sapiens
  source 1..58: mol_type = genomic DNA
tcctaaccaagtgtctctttcccccagaatggctgcagtctgtccaggccaccatgat

SEQ ID NO: 77 - DNA, 25 bp, Homo sapiens
  source 1..25: mol_type = other DNA
aggattggttcacagagaaaggggg

SEQ ID NO: 78 - DNA, 25 bp, Homo sapiens
  source 1..25: mol_type = other DNA
ggattggttcacagagaaagggggt

SEQ ID NO: 79 - DNA, 25 bp, Homo sapiens
  source 1..25: mol_type = other DNA
gattggttcacagagaaagggggtc

SEQ ID NO: 80 - DNA, 25 bp, Homo sapiens
  source 1..25: mol_type = other DNA
attggttcacagagaaagggggtct

SEQ ID NO: 81 - DNA, 25 bp, Homo sapiens
  source 1..25: mol_type = other DNA
ttggttcacagagaaagggggtctt

SEQ ID NO: 82 - DNA, 25 bp, Homo sapiens
  source 1..25: mol_type = other DNA
tggttcacagagaaagggggtctta

SEQ ID NO: 83 - DNA, 25 bp, Homo sapiens
  source 1..25: mol_type = other DNA
ggttcacagagaaagggggtcttac

SEQ ID NO: 84 - DNA, 25 bp, Homo sapiens
  source 1..25: mol_type = other DNA
gttcacagagaaagggggtcttacc

SEQ ID NO: 85 - DNA, 25 bp, Homo sapiens
  source 1..25: mol_type = other DNA
ttcacagagaaagggggtcttaccg

SEQ ID NO: 86 - DNA, 25 bp, Homo sapiens
  source 1..25: mol_type = other DNA
tcacagagaaagggggtcttaccga

SEQ ID NO: 87 - DNA, 25 bp, Homo sapiens
  source 1..25: mol_type = other DNA
cacagagaaagggggtcttaccgac

SEQ ID NO: 88 - DNA, 25 bp, Homo sapiens
  source 1..25: mol_type = other DNA
acagagaaagggggtcttaccgacg

SEQ ID NO: 89 - DNA, 25 bp, Homo sapiens
  source 1..25: mol_type = other DNA
cagagaaagggggtcttaccgacgt

SEQ ID NO: 90 - DNA, 25 bp, Homo sapiens
  source 1..25: mol_type = other DNA
agagaaagggggtcttaccgacgtc

SEQ ID NO: 91 - DNA, 25 bp, Homo sapiens
  source 1..25: mol_type = other DNA
gagaaagggggtcttaccgacgtca

SEQ ID NO: 92 - DNA, 25 bp, Homo sapiens
  source 1..25: mol_type = other DNA
agaaagggggtcttaccgacgtcag

SEQ ID NO: 93 - DNA, 25 bp, Homo sapiens
  source 1..25: mol_type = other DNA
gaaagggggtcttaccgacgtcaga

SEQ ID NO: 94 - DNA, 25 bp, Homo sapiens
  source 1..25: mol_type = other DNA
aaagggggtcttaccgacgtcagac

SEQ ID NO: 95 - DNA, 25 bp, Homo sapiens
  source 1..25: mol_type = other DNA
aagggggtcttaccgacgtcagaca

SEQ ID NO: 96 - DNA, 25 bp, Homo sapiens
  source 1..25: mol_type = other DNA
agggggtcttaccgacgtcagacag

SEQ ID NO: 97 - DNA, 25 bp, Homo sapiens
  source 1..25: mol_type = other DNA
gggggtcttaccgacgtcagacagg

SEQ ID NO: 98 - DNA, 25 bp, Homo sapiens
  source 1..25: mol_type = other DNA
ggggtcttaccgacgtcagacaggt

SEQ ID NO: 99 - DNA, 25 bp, Homo sapiens
  source 1..25: mol_type = other DNA
gggtcttaccgacgtcagacaggtc

SEQ ID NO: 100 - DNA, 25 bp, Homo sapiens
  source 1..25: mol_type = other DNA
ggtcttaccgacgtcagacaggtcc

SEQ ID NO: 101 - DNA, 25 bp, Homo sapiens
  source 1..25: mol_type = other DNA
gtcttaccgacgtcagacaggtccg

SEQ ID NO: 102 - DNA, 25 bp, Homo sapiens
  source 1..25: mol_type = other DNA
tcttaccgacgtcagacaggtccgg

SEQ ID NO: 103 - DNA, 25 bp, Homo sapiens
  source 1..25: mol_type = other DNA
cttaccgacgtcagacaggtccggt

SEQ ID NO: 104 - DNA, 25 bp, Homo sapiens
  source 1..25: mol_type = other DNA
ttaccgacgtcagacaggtccggtg

SEQ ID NO: 105 - DNA, 25 bp, Homo sapiens
  source 1..25: mol_type = other DNA
taccgacgtcagacaggtccggtgg

SEQ ID NO: 106 - DNA, 25 bp, Homo sapiens
  source 1..25: mol_type = other DNA
accgacgtcagacaggtccggtggt

SEQ ID NO: 107 - DNA, 25 bp, Homo sapiens
  source 1..25: mol_type = other DNA
ccgacgtcagacaggtccggtggta

SEQ ID NO: 108 - DNA, 25 bp, Homo sapiens
  source 1..25: mol_type = other DNA
cgacgtcagacaggtccggtggtac

SEQ ID NO: 109 - DNA, 25 bp, Homo sapiens
  source 1..25: mol_type = other DNA
gacgtcagacaggtccggtggtact

SEQ ID NO: 110 - DNA, 25 bp, Homo sapiens
  source 1..25: mol_type = other DNA
acgtcagacaggtccggtggtacta

SEQ ID NO: 111 - DNA, 58 bp, Homo sapiens
  source 1..58: mol_type = genomic DNA
tacatcactggaatcttccaaattcttgctggtaagttgtggatggtaaagtccatgt

SEQ ID NO: 112 - DNA, 25 bp, Homo sapiens
  source 1..25: mol_type = other DNA
atgtagtgaccttagaaggtttaag

SEQ ID NO: 113 - DNA, 25 bp, Homo sapiens
  source 1..25: mol_type = other DNA
tgtagtgaccttagaaggtttaaga

SEQ ID NO: 114 - DNA, 25 bp, Homo sapiens
  source 1..25: mol_type = other DNA
gtagtgaccttagaaggtttaagaa

SEQ ID NO: 115 - DNA, 25 bp, Homo sapiens
  source 1..25: mol_type = other DNA
tagtgaccttagaaggtttaagaac

SEQ ID NO: 116 - DNA, 25 bp, Homo sapiens
  source 1..25: mol_type = other DNA
agtgaccttagaaggtttaagaacg

SEQ ID NO: 117 - DNA, 25 bp, Homo sapiens
  source 1..25: mol_type = other DNA
gtgaccttagaaggtttaagaacga

SEQ ID NO: 118 - DNA, 25 bp, Homo sapiens
  source 1..25: mol_type = other DNA
tgaccttagaaggtttaagaacgac

SEQ ID NO: 119 - DNA, 25 bp, Homo sapiens
  source 1..25: mol_type = other DNA
gaccttagaaggtttaagaacgacc

SEQ ID NO: 120 - DNA, 25 bp, Homo sapiens
  source 1..25: mol_type = other DNA
accttagaaggtttaagaacgacca

SEQ ID NO: 121 - DNA, 25 bp, Homo sapiens
  source 1..25: mol_type = other DNA
ccttagaaggtttaagaacgaccat

SEQ ID NO: 122 - DNA, 25 bp, Homo sapiens
  source 1..25: mol_type = other DNA
cttagaaggtttaagaacgaccatt

SEQ ID NO: 123 - DNA, 25 bp, Homo sapiens
  source 1..25: mol_type = other DNA
ttagaaggtttaagaacgaccattc

SEQ ID NO: 124 - DNA, 25 bp, Homo sapiens
  source 1..25: mol_type = other DNA
tagaaggtttaagaacgaccattca

SEQ ID NO: 125 - DNA, 25 bp, Homo sapiens
  source 1..25: mol_type = other DNA
agaaggtttaagaacgaccattcaa

SEQ ID NO: 126 - DNA, 25 bp, Homo sapiens
  source 1..25: mol_type = other DNA
gaaggtttaagaacgaccattcaac

SEQ ID NO: 127 - DNA, 25 bp, Homo sapiens
  source 1..25: mol_type = other DNA
aaggtttaagaacgaccattcaaca

SEQ ID NO: 128 - DNA, 25 bp, Homo sapiens
  source 1..25: mol_type = other DNA
aggtttaagaacgaccattcaacac

SEQ ID NO: 129 - DNA, 25 bp, Homo sapiens
  source 1..25: mol_type = other DNA
ggtttaagaacgaccattcaacacc

SEQ ID NO: 130 - DNA, 25 bp, Homo sapiens
  source 1..25: mol_type = other DNA
gtttaagaacgaccattcaacacct

SEQ ID NO: 131 - DNA, 25 bp, Homo sapiens
  source 1..25: mol_type = other DNA
tttaagaacgaccattcaacaccta

SEQ ID NO: 132 - DNA, 25 bp, Homo sapiens
  source 1..25: mol_type = other DNA
ttaagaacgaccattcaacacctac

SEQ ID NO: 133 - DNA, 25 bp, Homo sapiens
  source 1..25: mol_type = other DNA
taagaacgaccattcaacacctacc

SEQ ID NO: 134 - DNA, 25 bp, Homo sapiens
  source 1..25: mol_type = other DNA
aagaacgaccattcaacacctacca

SEQ ID NO: 135 - DNA, 25 bp, Homo sapiens
  source 1..25: mol_type = other DNA
agaacgaccattcaacacctaccat

SEQ ID NO: 136 - DNA, 25 bp, Homo sapiens
  source 1..25: mol_type = other DNA
gaacgaccattcaacacctaccatt

SEQ ID NO: 137 - DNA, 25 bp, Homo sapiens
  source 1..25: mol_type = other DNA
aacgaccattcaacacctaccattt

SEQ ID NO: 138 - DNA, 25 bp, Homo sapiens
  source 1..25: mol_type = other DNA
acgaccattcaacacctaccatttc

SEQ ID NO: 139 - DNA, 25 bp, Homo sapiens
  source 1..25: mol_type = other DNA
cgaccattcaacacctaccatttca

SEQ ID NO: 140 - DNA, 25 bp, Homo sapiens
  source 1..25: mol_type = other DNA
gaccattcaacacctaccatttcag

SEQ ID NO: 141 - DNA, 25 bp, Homo sapiens
  source 1..25: mol_type = other DNA
accattcaacacctaccatttcagg

SEQ ID NO: 142 - DNA, 25 bp, Homo sapiens
  source 1..25: mol_type = other DNA
ccattcaacacctaccatttcaggt

SEQ ID NO: 143 - DNA, 25 bp, Homo sapiens
  source 1..25: mol_type = other DNA
cattcaacacctaccatttcaggta

SEQ ID NO: 144 - DNA, 25 bp, Homo sapiens
  source 1..25: mol_type = other DNA
attcaacacctaccatttcaggtac

SEQ ID NO: 145 - DNA, 25 bp, Homo sapiens
  source 1..25: mol_type = other DNA
ttcaacacctaccatttcaggtaca

SEQ ID NO: 146 - DNA, 21 bp, Homo sapiens
  source 1..21: mol_type = other DNA
cacagagaaagggggtcttac

SEQ ID NO: 147 - DNA, 20 bp, Homo sapiens
  source 1..20: mol_type = other DNA
aaagggggtcttaccgacgt

SEQ ID NO: 148 - DNA, 20 bp, Homo sapiens
  source 1..20: mol_type = other DNA
ggtcttaccgacgtcagaca

SEQ ID NO: 149 - DNA, 21 bp, Homo sapiens
  source 1..21: mol_type = other DNA
gaacgaccattcaacacctac

SEQ ID NO: 150 - DNA, 20 bp, Homo sapiens
  source 1..20: mol_type = other DNA
cgaccattcaacacctacca

SEQ ID NO: 151 - DNA, 20 bp, Homo sapiens
  source 1..20: mol_type = other DNA
cattcaacacctaccatttc

SEQ ID NO: 152 - DNA, 35 bp, Homo sapiens
  source 1..35: mol_type = genomic DNA
tctgattctctctagcaatggatcgtgggcaatgg

SEQ ID NO: 153 - DNA, 30 bp, Homo sapiens
  source 1..30: mol_type = genomic DNA
catcatcaccaaacggtgaggctggttttg

SEQ ID NO: 154 - DNA, 13 bp, Homo sapiens
  source 1..13: mol_type = genomic DNA
ctagcaatggatc

SEQ ID NO: 155 - DNA, 11 bp, Homo sapiens
  source 1..11: mol_type = genomic DNA
cggtgaggctg

SEQ ID NO: 156 - DNA, 24 bp, Homo sapiens
  source 1..24: mol_type = other DNA
gatcgttacctaggccactccgac

SEQ ID NO: 157 - DNA, 13 bp, Homo sapiens
  source 1..13: mol_type = genomic DNA
gcaatggatcgtg

SEQ ID NO: 158 - DNA, 10 bp, Homo sapiens
  source 1..10: mol_type = genomic DNA
caccaaacgg

SEQ ID NO: 159 - DNA, 23 bp, Homo sapiens
  source 1..23: mol_type = other DNA
cgttacctagcacgtggtttgcc

SEQ ID NO: 160 - DNA, 13 bp, Homo sapiens
  source 1..13: mol_type = genomic DNA
ctctctagcaatg

SEQ ID NO: 161 - DNA, 11 bp, Homo sapiens
  source 1..11: mol_type = genomic DNA
caaacggtgag

SEQ ID NO: 162 - DNA, 24 bp, Homo sapiens
  source 1..24: mol_type = other DNA
gagagatcgttacgtttgccactc

SEQ ID NO: 163 - DNA, 58 bp, Homo sapiens
  source 1..58: mol_type = other DNA
gcctcacgcccagcgctctcctcgcaggcagaaactccgctgagcagaacttgccgcc

SEQ ID NO: 164 - DNA, 25 bp, Homo sapiens
  source 1..25: mol_type = other DNA
cggagtgcgggtcgcgagaggagcg

SEQ ID NO: 165 - DNA, 25 bp, Homo sapiens
  source 1..25: mol_type = other DNA
ggagtgcgggtcgcgagaggagcgt

SEQ ID NO: 166 - DNA, 25 bp, Homo sapiens
  source 1..25: mol_type = other DNA
gagtgcgggtcgcgagaggagcgtc

SEQ ID NO: 167 - DNA, 25 bp, Homo sapiens
  source 1..25: mol_type = other DNA
agtgcgggtcgcgagaggagcgtcc

SEQ ID NO: 168 - DNA, 25 bp, Homo sapiens
  source 1..25: mol_type = other DNA
gtgcgggtcgcgagaggagcgtccg

SEQ ID NO: 169 - DNA, 25 bp, Homo sapiens
  source 1..25: mol_type = other DNA
tgcgggtcgcgagaggagcgtccgt

SEQ ID NO: 170 - DNA, 25 bp, Homo sapiens
  source 1..25: mol_type = other DNA
gcgggtcgcgagaggagcgtccgtc

SEQ ID NO: 171 - DNA, 25 bp, Homo sapiens
  source 1..25: mol_type = other DNA
cgggtcgcgagaggagcgtccgtct

SEQ ID NO: 172 - DNA, 25 bp, Homo sapiens
  source 1..25: mol_type = other DNA
gggtcgcgagaggagcgtccgtctt

SEQ ID NO: 173 - DNA, 25 bp, Homo sapiens
  source 1..25: mol_type = other DNA
ggtcgcgagaggagcgtccgtcttt

SEQ ID NO: 174 - DNA, 25 bp, Homo sapiens
  source 1..25: mol_type = other DNA
gtcgcgagaggagcgtccgtctttg

SEQ ID NO: 175 - DNA, 25 bp, Homo sapiens
  source 1..25: mol_type = other DNA
tcgcgagaggagcgtccgtctttga

SEQ ID NO: 176 - DNA, 25 bp, Homo sapiens
  source 1..25: mol_type = other DNA
cgcgagaggagcgtccgtctttgag

SEQ ID NO: 177 - DNA, 25 bp, Homo sapiens
  source 1..25: mol_type = other DNA
gcgagaggagcgtccgtctttgagg

SEQ ID NO: 178 - DNA, 25 bp, Homo sapiens
  source 1..25: mol_type = other DNA
cgagaggagcgtccgtctttgaggc

SEQ ID NO: 179 - DNA, 25 bp, Homo sapiens
  source 1..25: mol_type = other DNA
gagaggagcgtccgtctttgaggcg

SEQ ID NO: 180 - DNA, 25 bp, Homo sapiens
  source 1..25: mol_type = other DNA
agaggagcgtccgtctttgaggcga

SEQ ID NO: 181 - DNA, 25 bp, Homo sapiens
  source 1..25: mol_type = other DNA
gaggagcgtccgtctttgaggcgac

SEQ ID NO: 182 - DNA, 25 bp, Homo sapiens
  source 1..25: mol_type = other DNA
aggagcgtccgtctttgaggcgact

SEQ ID NO: 183 - DNA, 25 bp, Homo sapiens
  source 1..25: mol_type = other DNA
ggagcgtccgtctttgaggcgactc

SEQ ID NO: 184 - DNA, 25 bp, Homo sapiens
  source 1..25: mol_type = other DNA
gagcgtccgtctttgaggcgactcg

SEQ ID NO: 185 - DNA, 25 bp, Homo sapiens
  source 1..25: mol_type = other DNA
agcgtccgtctttgaggcgactcgt

SEQ ID NO: 186 - DNA, 25 bp, Homo sapiens
  source 1..25: mol_type = other DNA
gcgtccgtctttgaggcgactcgtc

SEQ ID NO: 187 - DNA, 25 bp, Homo sapiens
  source 1..25: mol_type = other DNA
cgtccgtctttgaggcgactcgtct

SEQ ID NO: 188 - DNA, 25 bp, Homo sapiens
  source 1..25: mol_type = other DNA
gtccgtctttgaggcgactcgtctt

SEQ ID NO: 189 - DNA, 25 bp, Homo sapiens
  source 1..25: mol_type = other DNA
tccgtctttgaggcgactcgtcttg

SEQ ID NO: 190 - DNA, 25 bp, Homo sapiens
  source 1..25: mol_type = other DNA
ccgtctttgaggcgactcgtcttga

SEQ ID NO: 191 - DNA, 25 bp, Homo sapiens
  source 1..25: mol_type = other DNA
cgtctttgaggcgactcgtcttgaa

SEQ ID NO: 192 - DNA, 25 bp, Homo sapiens
  source 1..25: mol_type = other DNA
gtctttgaggcgactcgtcttgaac

SEQ ID NO: 193 - DNA, 25 bp, Homo sapiens
  source 1..25: mol_type = other DNA
tctttgaggcgactcgtcttgaacg

SEQ ID NO: 194 - DNA, 25 bp, Homo sapiens
  source 1..25: mol_type = other DNA
ctttgaggcgactcgtcttgaacgg

SEQ ID NO: 195 - DNA, 25 bp, Homo sapiens
  source 1..25: mol_type = other DNA
tttgaggcgactcgtcttgaacggc

SEQ ID NO: 196 - DNA, 25 bp, Homo sapiens
  source 1..25: mol_type = other DNA
ttgaggcgactcgtcttgaacggcg

SEQ ID NO: 197 - DNA, 25 bp, Homo sapiens
  source 1..25: mol_type = other DNA
tgaggcgactcgtcttgaacggcgg

SEQ ID NO: 198 - DNA, 58 bp, Homo sapiens
  source 1..58: mol_type = genomic DNA
ggtgctgctgttcgtctccacgatcgtcagcgtgagtgcctggcggggaggctccctg

SEQ ID NO: 199 - DNA, 25 bp, Homo sapiens
  source 1..25: mol_type = other DNA
ccacgacgacaagcagaggtgctag

SEQ ID NO: 200 - DNA, 25 bp, Homo sapiens
  source 1..25: mol_type = other DNA
cacgacgacaagcagaggtgctagc

SEQ ID NO: 201 - DNA, 25 bp, Homo sapiens
  source 1..25: mol_type = other DNA
acgacgacaagcagaggtgctagca

SEQ ID NO: 202 - DNA, 25 bp, Homo sapiens
  source 1..25: mol_type = other DNA
cgacgacaagcagaggtgctagcag

SEQ ID NO: 203 - DNA, 25 bp, Homo sapiens
  source 1..25: mol_type = other DNA
gacgacaagcagaggtgctagcagt

SEQ ID NO: 204 - DNA, 25 bp, Homo sapiens
  source 1..25: mol_type = other DNA
acgacaagcagaggtgctagcagtc

SEQ ID NO: 205 - DNA, 25 bp, Homo sapiens
  source 1..25: mol_type = other DNA
cgacaagcagaggtgctagcagtcg

SEQ ID NO: 206 - DNA, 25 bp, Homo sapiens
  source 1..25: mol_type = other DNA
gacaagcagaggtgctagcagtcgc

SEQ ID NO: 207 - DNA, 25 bp, Homo sapiens
  source 1..25: mol_type = other DNA
acaagcagaggtgctagcagtcgca

SEQ ID NO: 208 - DNA, 25 bp, Homo sapiens
  source 1..25: mol_type = other DNA
caagcagaggtgctagcagtcgcac

SEQ ID NO: 209 - DNA, 25 bp, Homo sapiens
  source 1..25: mol_type = other DNA
aagcagaggtgctagcagtcgcact

SEQ ID NO: 210 - DNA, 25 bp, Homo sapiens
  source 1..25: mol_type = other DNA
agcagaggtgctagcagtcgcactc

SEQ ID NO: 211 - DNA, 25 bp, Homo sapiens
  source 1..25: mol_type = other DNA
gcagaggtgctagcagtcgcactca

SEQ ID NO: 212 - DNA, 25 bp, Homo sapiens
  source 1..25: mol_type = other DNA
cagaggtgctagcagtcgcactcac

SEQ ID NO: 213 - DNA, 25 bp, Homo sapiens
  source 1..25: mol_type = other DNA
agaggtgctagcagtcgcactcacg

SEQ ID NO: 214 - DNA, 25 bp, Homo sapiens
  source 1..25: mol_type = other DNA
gaggtgctagcagtcgcactcacgg

SEQ ID NO: 215 - DNA, 25 bp, Homo sapiens
  source 1..25: mol_type = other DNA
aggtgctagcagtcgcactcacgga

SEQ ID NO: 216 - DNA, 25 bp, Homo sapiens
  source 1..25: mol_type = other DNA
ggtgctagcagtcgcactcacggac

SEQ ID NO: 217 - DNA, 25 bp, Homo sapiens
  source 1..25: mol_type = other DNA
gtgctagcagtcgcactcacggacc

SEQ ID NO: 218 - DNA, 25 bp, Homo sapiens
  source 1..25: mol_type = other DNA
tgctagcagtcgcactcacggaccg

SEQ ID NO: 219 - DNA, 25 bp, Homo sapiens
  source 1..25: mol_type = other DNA
gctagcagtcgcactcacggaccgc

SEQ ID NO: 220 - DNA, 25 bp, Homo sapiens
  source 1..25: mol_type = other DNA
ctagcagtcgcactcacggaccgcc

SEQ ID NO: 221 - DNA, 25 bp, Homo sapiens
  source 1..25: mol_type = other DNA
tagcagtcgcactcacggaccgccc

SEQ ID NO: 222 - DNA, 25 bp, Homo sapiens
  source 1..25: mol_type = other DNA
agcagtcgcactcacggaccgcccc

SEQ ID NO: 223 - DNA, 25 bp, Homo sapiens
  source 1..25: mol_type = other DNA
gcagtcgcactcacggaccgcccct

SEQ ID NO: 224 - DNA, 25 bp, Homo sapiens
  source 1..25: mol_type = other DNA
cagtcgcactcacggaccgcccctc

SEQ ID NO: 225 - DNA, 25 bp, Homo sapiens
  source 1..25: mol_type = other DNA
agtcgcactcacggaccgcccctcc

SEQ ID NO: 226 - DNA, 25 bp, Homo sapiens
  source 1..25: mol_type = other DNA
gtcgcactcacggaccgcccctccg

SEQ ID NO: 227 - DNA, 25 bp, Homo sapiens
  source 1..25: mol_type = other DNA
tcgcactcacggaccgcccctccga

SEQ ID NO: 228 - DNA, 25 bp, Homo sapiens
  source 1..25: mol_type = other DNA
cgcactcacggaccgcccctccgag

SEQ ID NO: 229 - DNA, 25 bp, Homo sapiens
  source 1..25: mol_type = other DNA
gcactcacggaccgcccctccgagg

SEQ ID NO: 230 - DNA, 25 bp, Homo sapiens
  source 1..25: mol_type = other DNA
cactcacggaccgcccctccgaggg

SEQ ID NO: 231 - DNA, 25 bp, Homo sapiens
  source 1..25: mol_type = other DNA
actcacggaccgcccctccgaggga

SEQ ID NO: 232 - DNA, 25 bp, Homo sapiens
  source 1..25: mol_type = other DNA
ctcacggaccgcccctccgagggac

SEQ ID NO: 233 - DNA, 13 bp, Homo sapiens
  source 1..13: mol_type = genomic DNA
gcaatggatcgtg

SEQ ID NO: 234 - DNA, 11 bp, Homo sapiens
  source 1..11: mol_type = genomic DNA
gtgaggctggt

SEQ ID NO: 235 - DNA, 24 bp, Homo sapiens
  source 1..24: mol_type = other DNA
cgttacctagcaccactccgacca

SEQ ID NO: 236 - DNA, 13 bp, Homo sapiens
  source 1..13: mol_type = genomic DNA
gattctctctagc

SEQ ID NO: 237 - DNA, 11 bp, Homo sapiens
  source 1..11: mol_type = genomic DNA
tcaccaaacgg

SEQ ID NO: 238 - DNA, 24 bp, Homo sapiens
  source 1..24: mol_type = other DNA
ctaagagagatcgagtggtttgcc

SEQ ID NO: 239 - DNA, 21 bp, Homo sapiens
  source 1..21: mol_type = other DNA
gaagaggtgctacagttctgc